(19) United States
(12) Patent Application Publication
(10) Pub. No.: US 2025/0213587 A1
(43) Pub. Date: Jul. 3, 2025

(54) ITK INHIBITORS FOR INCREASING TH1 CELL ACTIVITY

(71) Applicant: Corvus Pharmaceuticals, Inc., Burlingame, CA (US)

(72) Inventors: Richard A. Miller, Burlingame, CA (US); Lih-Yun Hsu, Burlingame, CA (US)

(21) Appl. No.: 18/852,735

(22) PCT Filed: Apr. 4, 2023
(86) PCT No.: PCT/US2023/017376

§ 371 Date: Sep. 30, 2024

Related U.S. Application Data
(60) Provisional application No. 63/418,158, filed on Oct. 21, 2022.
(60) Provisional application No. 63/327,563, filed on Apr. 5, 2022.

(51) Int. Cl. A61K 31/551; A61K 31/496; A61P 1/00; A61P 11/00; A61P 35/02

(52) U.S. Cl. CPC A61K 31/551; A61K 31/496; A61P 1/00; A61P 11/00; A61P 35/02

(57) ABSTRACT

The disclosure provides, inter alia, pharmaceutical compositions comprising IL-2-inducible T-cell kinase (ITK) inhibitors and methods to increase Th1 cell activity, treat cancer, treat autoimmune diseases, treat allergies, and reverse T cell exhaustion by administering to patients effective amounts of IL-2-inducible T-cell kinase (ITK) inhibitors.

### CROSS-REFERENCE TO RELATED APPLICATIONS

This application claims priority to U.S. Application No. 63/418,158 filed Oct. 21, 2022, and U.S. Application No. 63/327,563 filed Apr. 5, 2022, the disclosures of which are incorporated by reference herein in their entirety.

### BACKGROUND

Interleukin 2 inducible T cell kinase (ITK) is a TEC family non receptor tyrosine kinase expressed in T cells with an important role in T cell receptor (TCR) signaling. TCR signaling regulates the development of T cells within the thymus, wherein the strength or duration of downstream signaling pathways determines the survival, maturation, and differentiation of thymocytes into mature T cells. ITK−/− mice exhibit defects in T helper 2 (Th2) differentiation while retaining the ability to differentiate into T helper 1 (Th1) cells and secrete IFNγ. Upon TCR stimulation, ITK is recruited to the membrane associated SLP 76/LAT adapter complex, where it is phosphorylated and activated by the src family kinase LCK. Activated ITK then phosphorylates PLCγ1, leading to the mobilization of Ca2+ and to the activation of growth and survival pathways including MAPK and NFκB. Another TEC-family kinase known as resting lymphocyte kinase (RLK/TXK) is also expressed in T cells and is similarly activated by TCR-driven phosphorylation by src-family kinases and interacts with many of the same signaling components as ITK. Whereas ITK^{-/-}CD4+ T cells in mice have impaired T cell activation and differentiation, ITK^{-/-}RLK^{-/-} double knockout T cells have a more substantial exacerbation of the signaling defect, and a profound loss of normal T cell function in mice. Thus, selective inhibition of ITK while sparing RLK may be necessary to therapeutically modulate Th responses without affecting overall T-dependent immunity. Transgenic mice expressing a kinase-dead ITK allele were protected from inflammatory symptoms when challenged with ovalbumin, supporting ITK kinase inhibition as a strategy for T cell-driven inflammation. There is a need in the art for the treatment of diseases modulated by Th responses. The present disclosure is directed to this as well as other important ends.

### BRIEF SUMMARY

Provided herein are methods of treating a patient having deficient Th1 activity by administering to the patient an ITK inhibitor at an effective amount to increase Th1 activity. In embodiments, the effective amount of the ITK inhibitor to increase Th1 activity is an amount that increases the number of Th1⁺ T cells; increases the ratio of Th1⁺ T cells to Th2⁺ T cells; increases the ratio of IFNγ⁺CD4⁺ T cells to IL-4⁺CD4⁺ T cells; increases IFNγ production; increases CD8+ cytotoxic lymphocytes; inhibits IL-4 production; inhibits IL-5 production; inhibits IL-13 production; inhibits Th2-secreted cytokines; decreases Th2+ cells; decreases Th17+ T cells; decreases eosinophils; or a combination of two or more thereof. In embodiments, the effective amount of the ITK inhibitor to increase Th1 activity is from about 0.6 mmole to about 1.6 mmole of the ITK inhibitor per day. In embodiments, the ITK inhibitor has a selectivity for ITK that is at least 50-fold greater than the selectivity for resting lymphocyte kinase. In embodiments, the ITK inhibitor is CPI-818.

Provided herein methods of treating a cancer, an autoimmune disease, or an allergy in a patient in need thereof by administering to the patient about 0.6 mmole per day to about 1.6 mmole per day of an ITK inhibitor. In embodiments, the ITK inhibitor has a selectivity for ITK that is at least 50-fold greater than the selectivity for resting lymphocyte kinase. In embodiments, the ITK inhibitor is CPI-818.

Provided herein are methods for treating a cancer, an autoimmune disease, or an allergy in a patient in need thereof by administering to the patient CPI-818 in an amount of about 250 mg to about 1,000 mg per day. In embodiments, a biological sample obtained from the patient has (i) an increased level of LAG3, (ii) an increased level of TIGIT, (iii) an increased level of PD-1, (iv) a decreased level of IFNγ, (v) a decreased level of granzyme B, or (vi) a combination of two or more of the foregoing, relative to a control.

Provided herein are methods of reversing T cell exhaustion in a patient by administering to the patient an effetive amount of an ITK inhibitor. In embodiments, the ITK inhibitor has a selectivity for ITK that is at least 50-fold greater than the selectivity for resting lymphocyte kinase. In embodiments, the ITK inhibitor is CPI-818. In embodiments, the patient is identified as having T cell exhaustion when a biological sample obtained from the patient has (i) an increased level of LAG3, (ii) an increased level of TIGIT, (iii) an increased level of PD-1, (iv) a decreased level of IFNγ, (v) a decreased level of granzyme B, or (vi) a combination of two or more of the foregoing, relative to a control.

These and other embodiments of the disclosure are provided in detail herein.

### BRIEF DESCRIPTION OF THE DRAWINGS

FIGS. 1A-1C show the structure of an ITK inhibitor and the activity thereof. FIGS. 1A-1B show the chemical structure of ITK inhibitors and FIG. 1C is a table showing biochemical and functional inhibition of ITK activity. IC₅₀ values are the mean of at least two technical replicates. Biochemical IC₅₀ data was obtained by microfluidic assay. All other values were obtained using the Lanthascreen format.

FIGS. 2A-2E show covalent irreversible inhibition of ITK by CPI-818. FIG. 2A: time-dependent inhibition of ITK by CPI-818. Thirteen concentrations of CPI-818 were assayed ranging between 10 μM and 2.4 nM. The curves are the best fit solutions to equation 3. FIG. 2B: A secondary plot of k_{obs} versus inhibitor concentration gives a hyperbola typical of an enzyme inactivator with a mechanism conforming to scheme 1 in FIG. 11. The curve is the best fit solution to equation 4 which gave k_{inact}/Kᵢ=1.37×10⁻² μM⁻¹s⁻¹ for CPI-818 inactivation of ITK and is the mean of three independent determinations. FIG. 2C: Dilution of BMS-509744 or CP-818-inhibited ITK mixtures into a solution of competitive probe demonstrated reversible (T_{1/2}=2.6 min) and irreversible inhibition, respectively. The curve is the best fit solution to equation 3. FIG. 2D: ITK enzymatic activity remained inhibited following addition of CP-818-inhibited ITK into a solution of substrate following extensive dialysis. FIG. 2E: Removal of the Michael acceptor in the Cys442Ala ITK mutant significantly reduced sensitivity to inhibition by CPI-818 compared to wildtype enzyme. The curves are the best fit solutions to equation 2 and gave values of IC₅₀=520 nM, IC₅₀=2.6 nM for Cys442Ala and WT enzymes, respectively.

FIG. 3 is a table showing unique nested chymotryptic peptide sequences present in CPI-818 treated ITK identified by deconvolution of mass spectra demonstrating an increased mass equal to the mass of CPI-818.

FIG. 4 shows the chemical stability of the CPI-818 acrylamide. The acrylamide of CPI-818 is stable at 37° C. in the presence of physiological glutathione concentrations with minimal loss at 3 h which is similar to the approved drug ibrutinib. Each value is the result of a single replicate.

FIGS. 5A-5D show that CPI-818 inhibits TCR signaling downstream of ITK and blocks IL-2 production. FIG. 5A: Jurkat T cells were stimulated with anti-CD3 for 30 seconds in the presence of increasing concentrations of CPI-818. Cell lysates were analyzed by immunoblotting with specific antibodies to total and phosphorylated PLCγ1 and ZAP-70. FIGS. 5B-5D: CPI-818 inhibition of ERK and S6 phosphorylation and IL-2 secretion. ERK and S6 phosphorylation was measured by flow cytometry following CD3/28-crosslinking in human PBMC. IC₅₀ values of 39 nM and 96 nM were obtained for ERK and S6, respectively where each data point is the mean of three donors. IL-2 was measured by AlphaLISA where Jurkat T cells were incubated with CPI-818, washed to remove the inhibitor, and stimulated for 18 hours with anti-CD3. A representative curve is shown from one experiment with the mean of technical duplicates plotted. A mean IC₅₀ of 136 nM was obtained from 15 independent determinations. The curves are the best fit solutions to equation 6.

FIGS. 6A-6B show turnover of ITK in Jurkat and primary human T cells. Data are shown from representative experiments for degradation and synthesis of ITK where each data point is the mean of technical duplicates in Jurkat cells (FIG. 6A) and primary human T cells (FIG. 6B). Half-lives for degradation and synthesis were within the range of 6.9 h to 8.2 h for both Jurkat and primary human T cells. The curves are the best fit solutions to single exponential equations for decay and association.

FIG. 7 shows in vivo ITK occupancy in mice. CPI-818 provided potent and durable inhibition of mouse splenocyte ITK yielding very high levels of enzyme occupancy following a single 50 mg/kg PO dose. Data points are the mean value of 4 (PK) and 6 (occupancy) animals.

FIGS. 8A-8B show selective ITK inhibition by CPI-818 skews naïve human T cells toward the Th-1 phenotype. FIG. 8A: CPI-818 (1 μM) induced Th1 skewing with a 2-fold increase in the ratio of IFNγ+CD4+ T cells to IL 4+CD4+ T cells in 12 normal donors. FIG. 8B: CPI-818 (≥1 μM) reduced the total number of cells while maintaining cell viability.

FIGS. 9A-9B show selective Inhibition of ITK preserved NK-mediated ADCC. FIG. 9A: Lymphocyte subsets (5 donors/cell type) were purified from the peripheral blood of 22 healthy donors. The expression of ITK and RLK, relative to the house keeping gene IPO8, were determined by qPCR. FIG. 9B: Peripheral blood NK cells from 7 healthy donors were co-cultured at a 10:1 ratio with anti-CD20 bound target cells (B-cell lymphoma line Jeko) and with inhibitors for 18 hours. Cell lysis was detected by viability dye using flow cytometry. The % dead target cells in samples treated with DMSO were set as the maximum lysis (100%). CPI-818 (ITK-specific), CP-1392 (RLK-specific), and CP-2193 (ITK/RLK dual inhibition) are covalent small molecule Tec kinase inhibitors.

FIGS. 10A-10E show the efficacy of CPI-818 treatment in a mouse model of colitis. C.B-17 SCID mice (10/group) began to have access to control or CPI-818-formulated (300 mg/kg/day) diet, with or without anti-IL-12/23 treatment, one week before receiving CD4⁺CD45RB^{hi} T cells from BALB/c mice. Body weight (FIG. 10A) was monitored during the life phase. On day 48, mice were sacrificed, and colon weight (FIG. 10B) and length (FIG. 10C) recorded. The distal section of each colon was fixed and subjected to histologic analysis. Infiltrating CD3+ T cells were enumerated (FIG. 10D) and histologic lesions scored (FIG. 10E). *The difference between control and CPI-818 treated groups for each of the four lesion categories was statistically significant (P=0.004, P<0.002, P<0.008, and P=0.037 from left to right).

FIG. 11 is a scheme showing the time-dependent irreversible inhibition of ITK.

FIG. 12 is a table showing the selectivity of CPI-818 toward ITK among the 11 kinases of the human kinome containing a conserved cysteine at a position homologous to Cys-442 of ITK. Selectivity in a functional autophosphorylation assay was measured for the five TEC family kinases and JAK3. The values are the mean of technical duplicates for both assays.

FIG. 13 shows the RLK selective and ITK/RLK dual selective inhibitors CP 1392 and CP-2193.

FIGS. 14A-14B show ITK active site occupancy. FIG. 14A: Occupancy was measured in Jurkat cells following treatment with CP-464 (37° C., 1 h). Each point is the mean of technical duplicates. FIG. 14B: The chemical structure of the biotinylated probe CP-613.

FIGS. 15A-15C show the PK/PD relationship of CPI-818 in dogs (QD dose) and mice (BID dose and chow formulation. FIG. 15A: Plasma CPI-818 and ITK occupancy in PBMC was measured in dogs after a single 5 mg/kg PO dose. Each data point is the mean from three animals. FIG. 15B: Plasma CPI-818 and splenocyte ITK occupancy was measured in mice after 50 mg/kg BID, PO dosing. Each data point is the mean from three (PK) and four (occupancy) animals. FIG. 15C: Plasma CPI-818 and splenocyte ITK occupancy was measured in mice after administration in chow formulated to deliver 300 mg/kg/day. Each data point is the mean from five animals for PK and occupancy measurements.

FIGS. 16A-16B show the effect of 1 μM CPI-818 on IFNγ (FIG. 16A) and IL-4 (FIG. 16B) production by human CD4⁺ T cells.

FIGS. 17A-17D show H&E-stained mouse proximal colons in naïve animal (FIG. 17A), untreated disease control animal displaying histological features of colitis (FIG. 17B), and animal treated daily with CPI-818 where normal tissue architecture and composition was preserved (FIG. 17C). Scale bar=400 μm. Prevention of inflammatory bowel disease can be seen by histology and reduction of colon weight (FIG. 17D). Positive control=anti-IL 12/23.

FIG. 18 shows the anti-proliferative effects of CPI-818 in peripheral blood of 3 human patients with cutaneous T cell lymphoma and circulating malignant Sezary cells and a healthy donor. With reference to the graphs, the upper line is normal CD8 cells, the middle line is normal CD4 cells, and the lower line is Sézary cells.

FIG. 19 is a table showing the in vivo CPI-818 plasma concentration at four different dose administrations to human patients with peripheral T cell lymphomas. In each case, the dose (100 mg, 200 mg, 400 mg, 600 mg) was administered to the human patient twice daily.

FIG. 20 shows that the best responders received a dose of 200 mg BID, and that the response to treatment was not a linear dose-response curve.

FIGS. 21A-21B shows the steady state peak (FIG. 21A) and the steady state trough (FIG. 21B) ITK occupancy for CPI-818. The peak occupancies are consistently high with mean values of at least 90%. The trough occupancies were dose-dependent, with about 90% at the 400 mg BID and 600 mg BID doses, but were variable by subjects.

FIGS. 22A-22B show the anti-tumor activity of CPI-818 evaluated in human clinical trials. FIG. 22A shows the anti-tumor activity of the 4 doses of CPI-818 evaluated in human clinical trials (100 mg BID, 200 mg BID, 400 mg BID, 600 mg BID). FIG. 22B shows the anti-tumor activity of the 200 mg BID dose of CPI-818 evaluated in human clinical trials. Each lane represents a patient with the length indicating time on treatment. A treatment cycle is 21 days and then the cycle is repeated until the patient experiences tumor progression or unacceptable toxicity. The best response occurred at the dose of 200 mg BID.

FIG. 23 shows treatment results for a patient having peripheral T cell lymphoma not otherwise specified (PTCL-NOS). The patient had stable disease for 7 cycles (i.e., C1D1, C4D1, C7D1 where each cycle as 21 days long), treatment was interrupted for 3 weeks (DH or drug holiday), and then treatment was resumed for 10 cycles. The patient achieved complete response in treatment cycle 17 after 1 year of treatment. The duration of the complete response was 19 months. PET is Deauville PET score; ND is not done; SD is stable disease; CR is complete response.

FIG. 24 shows results for a patient with peripheral T cell lymphoma not otherwise specified (PTCL-NOS) treated with CPI-818 after 8 days and after 15 days. The patient exhibited a dramatic reduction of subcutaneous (SQ) tumor and improvement in platelets (PLT) and lactate dehydrogenase (LDH) within 8-15 days from the start of treatment. The eosinophil counts, which were markedly elevated decreased while on therapy.

FIGS. 25A-25B show that CPI-818 induces Th1 skewing and reduces eosinophils. CPI-818 increased Th1 cells with a concomitant tumor response and a decrease in Th17 pro-inflammatory cells (FIG. 25A) and provided for a marked reduction in hypereosinophilia (FIG. 25B). These findings are consistent with inhibitory effects of CPI-818 on tumor and/or normal Th2 cells.

FIGS. 26A-26B show that CPI-818 treatment increases in Th1 cells (FIG. 26A) and CD4+ effector cells (FIG. 26B) in blood and tumor of a patient with peripheral T cell lymphoma. In FIG. 26A, the upper point on day 84 is Th1 cells in tumor and the lower point is Th1 cells in blood. In FIG. 26B, the upper point on day 84 is CD4 effector cells in blood and the lower point is CD4 effector cells in tumor. CPI-818 treatment was oral administration of 200 mg of CPI-818 twice daily (BID).

FIG. 27 shows the effects of CPI-818 treatment on Th1/Th2 in a patient with peripheral T cell lymphoma, noting the 4 week abstinence from treatment starting at week 23. CPI-818 treatment was oral administration of 200 mg of CPI-818 twice daily (BID), except for the 4 weeks of abstinence. The patient's skin lesions starting to respond to treatment after CPI-818 was re-started following the 4 weeks of abstinence from treatment.

FIGS. 28A-28C show that CPI-818 inhibits Th2 cytokine production in vitro. FIG. 28A shows cytokine production in CD4+ cells from 3 normal subjects at varying concentrations of CPI-818 (μM). FIG. 28B shows cytokine production in Sézary cells from 2 subjects at varying concentrations of CPI-818 (μM). In brief, the normal CD4+ cells and Sezary cells were stimulated with anti-CD3/28/2 in the presence of varying concentrations of CPI-818, and the cytokines secreted in the supernatant were measured by immunoassay. The findings indicate that CPI-818 blocks the cytokines that are secreted by Th2 cells. This is significant because cytokines secreted by Th2 cells are involved in inflammation, allergy and autoimmunity. Interferon gamma (IFNg) is made by Th1 and is only inhibited at high concentrations of CPI-818. High concentrations of CPI-818 cause general T cell inhibition of proliferation/function, while intermediate concentrations of CPI-818 affect differentiation. In FIG. 28C, human peripheral blood CD4+ T cells were stimulated with anti-CD3/28/2, and there was a dose-dependent inhibition of Th2 cytokine observed. There was no effect on Th1-dependent IFNg, except at high concentrations. Inhibition of Th2 cells blocks production of various inflammatory cytokines, including IL-4, IL-5, IL-9, IL-13, and IL17a.

FIGS. 29A-29B show that CPI-818 inhibits lymphadenopathy (FIG. 29A) and proteinuria (FIG. 29B) in mouse MRL lymphoproliferation strain (a.k.a. MRL/lpr^{-/-}) lupus model.

FIG. 30 shows that CPI-818 significantly reduced skin thickening and dermal inflammation in an imiquimod-induced model of psoriasis.

FIGS. 31A-31D show that CPI-818 is effective in animal models of pulmonary fibrosis. FIGS. 31A-31B show that CPI-818 reduced lung weight and BALF leukocytes in a bleomycin-induced mouse pulmonary fibrosis model. FIG. 31C shows that CPI-818 reduced the Ashcroft score comparable to (at 10 mg/kg) or better than (at 30 mg/kg) nintadenib (OFEV® by Boehringer Ingelheim Pharmaceuticals), an FDA-approved drug for the treatment of idiopathic pulmonary fibrosis. FIG. 31D shows the plasma concentration of CPI-818 in mice at doses of 10 mg/mL and 30 mg/mL. In FIG. 31A: *p=0.003, **p=0.002, ***p=0.0001 compared to G2 placebo. In FIG. 31B: *p=0.01 compared to G2 placebo. In FIG. 31C: *p=0.004, **p=0.0008, ***p=0.005 compared to G2 placebo.

FIG. 32 shows that various concentrations of CPI-818 downregulate biomarkers of T cell exhaustion (i.e., LAG3, TIGIT, and PD-1) in human CD4 T cells.

FIGS. 33A-33B show that CPI-818 treatment reverses T cell exhaustion by increasing levels of granzyme B (FIG. 33A) and IFNγ (FIG. 33B).

### DETAILED DESCRIPTION

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

The terms “interleukin-2-inducible T-cell kinase” and “ITK” refer to a protein (including homologs, isoforms, and functional fragments thereof) with interleukin-2-inducible T-cell kinase activity. The term includes any recombinant or naturally-occurring form of ITK or variants thereof that maintain ITK activity (e.g. within at least 60%, 70%, 80%, 90%, or 100% activity compared to wildtype ITK). In aspects, the interleukin-2-inducible T-cell kinase protein encoded by the ITK gene has the amino acid sequence set forth in or corresponding to Entrez 3702, UniProt Q08881, or RefSeq (protein) NP_005537. In aspects, the ITK gene has the nucleic acid sequence set forth in RefSeq (mRNA) NM_005546. In aspects, the sequence corresponds to GI: 15718680, to NP_005537.3, to NM_005546.3, or to GI: 21614549.

The terms “Tec kinase” and “Tec kinase family” refer to a protein family (including homologs, isoforms, and functional fragments thereof) of non-receptor protein tyrosine kinases including the proteins TEC, BTK (Bruton's Tyrosine Kinase), ITK/EMT/TSK, BMX, and TXK/RLK. The term includes any recombinant or naturally-occurring form of a Tec family kinase or variant thereof that maintains Tec family kinase activity (e.g. within at least 50%, 60%, 70%, 80%, 90%, or 100% activity compared to wildtype Tec family kinase).

The term “inhibition,” “inhibit,” “inhibiting” and the like in reference to a protein-inhibitor interaction means negatively affecting (e.g. decreasing) the activity or function of the protein relative to the activity or function of the protein in the absence of the inhibitor. In embodiments, inhibition means negatively affecting (e.g. decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The term “LAG3” or “lymphocyte activation gene 3 protein” refer to a protein (including homologs, isoforms, and functional fragments thereof) with LAG3 activity. The term includes any recombinant or naturally-occurring form of LAG3 or variants thereof that maintain LAG3 activity (e.g. within at least 60%, 70%, 80%, 90%, or 100% activity compared to wildtype LAG3). In aspects, the LAG3 protein has the amino acid sequence set forth in or corresponding to UniProt P18627. In aspects, the LAG3 gene has the nucleic acid sequence set forth in NCBI Gene ID No. 3902.

The term “TIGIT” or “T-cell immunoreceptor with Ig and ITIM domains” refer to a protein (including homologs, isoforms, and functional fragments thereof) with TIGIT activity. The term includes any recombinant or naturally-occurring form of TIGIT or variants thereof that maintain TIGIT activity (e.g. within at least 60%, 70%, 80%, 90%, or 100% activity compared to wildtype TIGIT). In aspects, the TIGIT protein has the amino acid sequence set forth in or corresponding to UniProt Q495A1. In aspects, the TIGIT gene has the nucleic acid sequence set forth in NCBI Gene ID No. 201633.

The term “PD-1” or “programmed cell death protein 1” refer to a protein (including homologs, isoforms, and functional fragments thereof) with PD-1 activity. The term includes any recombinant or naturally-occurring form of PD-1 or variants thereof that maintain PD-1 activity (e.g. within at least 60%, 70%, 80%, 90%, or 100% activity compared to wildtype TIGIT). In aspects, the PD-1 protein encoded by the PDCD1 gene has the amino acid sequence set forth in or corresponding to UniProt Q15116. In aspects, the PDCD1 gene has the nucleic acid sequence set forth in NCBI Gene ID No. 5133.

“CPI-818” refers to the compound of Formula (A) or a pharmaceutically acceptable salt thereof. In embodiments, CPI-818 is in the form of the free base. CPI-818 and the compound of Formula (A) have the structure:

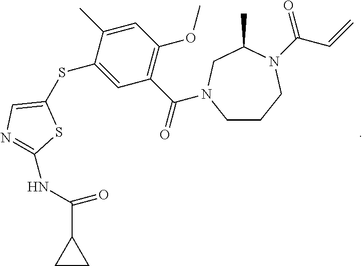 [SMILES: C=CC(=O)N1CCCN(C(=O)c2cc(Sc3cnc(NC(=O)C4CC4)s3)c(C)cc2OC)C[C@H]1C]

The abbreviations used herein have their conventional meaning within the chemical and biological arts. The chemical structures and formulae set forth herein are constructed according to the standard rules of chemical valency known in the chemical arts. Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbon atoms (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom, and wherein the nitrogen and sulfur atoms may optionally be oxidized, and the nitrogen heteroatom may optionally be quaternized). The heteroatom(s) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂NH—S(O)—CH₃, —CH₂—CH₂—S(O)₂—CH₃, —CH═CH—O—CH₃, —Si(CH₃)₃, —CH₂—CH═N—OCH₃, —CH—CH—N(CH₃)—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom. A heteroalkyl moiety may include two optionally different heteroatoms. A heteroalkyl moiety may include three optionally different heteroatoms. A heteroalkyl moiety may include four optionally different heteroatoms. A heteroalkyl moiety may include five optionally different heteroatoms. A heteroalkyl moiety may include up to 8 optionally different heteroatoms.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′-represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. In embodiments, a cycloalkyl is a spirocyclic cycloalkyl, wherein the spirocyclic rings are cycloalkyl rings. In embodiments, a cycloalkyl is a fused ring cycloalkyl, wherein the fused rings are cycloalkyl rings. In embodiments, a cycloalkyl is a bridged ring cycloalkyl, wherein the bridged rings are cycloalkyl rings. In embodiments, a bridged ring cycloalkyl refers to

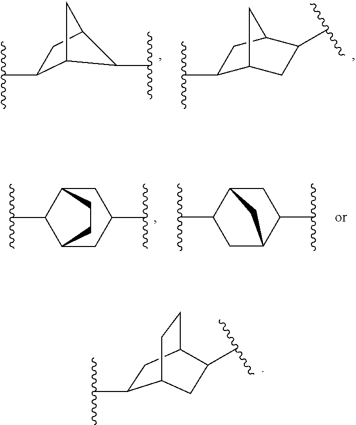

Non-limiting examples of a bridged ring heterocycloalkyl include

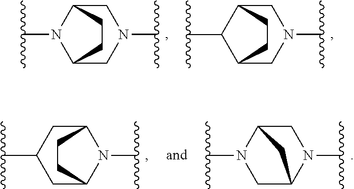

In embodiments, a cycloalkyl is monocyclic. In embodiments, a cycloalkyl is two rings. In embodiments, a cycloalkyl is three rings. In embodiments, a cycloalkyl is four rings. In embodiments, a cycloalkyl is five rings. In embodiments, a cycloalkyl is polycyclic. In embodiments, a heterocycloalkyl is a spirocyclic heterocycloalkyl, wherein the spirocyclic rings are one or more heterocycloalkyl rings and optionally one or more cycloalkyl rings. For example, spirocyclic heterocycloalkyl may refer to

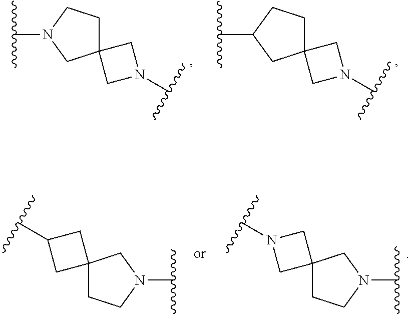 [SMILES: *N1CCC2(C1)CN(*)C2 ; *C1CCC2(C1)CN(*)C2 ; *C1CC2(CCN(*)C2)C1 ; *N1CCC2(C1)CN(*)C2]

In embodiments, a heterocycloalkyl is a fused ring heterocycloalkyl, wherein the fused rings are one or more heterocycloalkyl rings and optionally one or more cycloalkyl rings. In embodiments, a fused ring heterocycloalkyl refers to

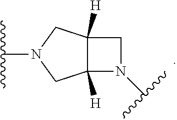 [SMILES: *N1C[C@@H]2CN(*)[C@@H]2C1]

In embodiments, a heterocycloalkyl is a bridged ring heterocycloalkyl, wherein the bridged rings are one or more heterocycloalkyl rings and optionally one or more cycloalkyl rings. In embodiments, the rings of a spirocyclic, fused ring, or bridged ring heterocycloalkyl are heterocyclic rings. In embodiments, a heterocycloalkyl is monocyclic. In embodiments, a heterocycloalkyl is two rings. In embodiments, a heterocycloalkyl is three rings. In embodiments, a heterocycloalkyl is four rings. In embodiments, a heterocycloalkyl is five rings. In embodiments, a heterocycloalkyl is polycyclic. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo (C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. In embodiments, an aryl is a fused ring aryl, wherein the fused rings are one or more aryl rings and optionally one or more cycloalkyl and/or heterocycloalkyl rings. In embodiments, an aryl is a bridged ring aryl, wherein the bridged rings are one or more aryl rings and optionally one or more cycloalkyl and/or heterocycloalkyl rings. In embodiments, the rings of a fused ring aryl or bridged ring aryl are aryl rings. In embodiments, an aryl is monocyclic. In embodiments, an aryl is two rings. In embodiments, an aryl is three rings. In embodiments, an aryl is four rings. In embodiments, an aryl is five rings. In embodiments, an aryl is polycyclic. In embodiments, a heteroaryl is a fused ring heteroaryl, wherein the fused rings are one or more heteroaryl rings and optionally one or more cycloalkyl, heterocycloalkyl, and/or aryl rings. In embodiments, a heteroaryl is a bridged ring heteroaryl, wherein the bridged rings are one or more heteroaryl rings and optionally one or more cycloalkyl, heterocycloalkyl, and/or aryl rings. In embodiments, the rings of a fused ring heteroaryl or bridged ring heteroaryl are heteroaryl rings. In embodiments, a heteroaryl is monocyclic. In embodiments, a heteroaryl is two rings. In embodiments, a heteroaryl is three rings. In embodiments, a heteroaryl is four rings. In embodiments, a heteroaryl is five rings. In embodiments, a heteroaryl is polycyclic. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O-bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” and “-” denote the point of attachment of a chemical moiety to the remainder of a molecule or chemical formula.

The term “oxo” means an oxygen that is double bonded to a carbon atom.

The term “alkylarylene” as an arylene moiety covalently bonded to an alkylene moiety (an alkylene linker). In embodiments, the alkylarylene group has the formula:

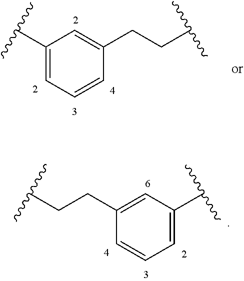 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —Cl₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)—NR″, —NR—C(NR′R″)═NR′″, —S(O) R′, —S(O)₂R′, —S(O)₂NR′R″, —NRSO₂R′, —NR′NR″R′″, —ONR′R″, —NR′C(O)NR″NR″R′″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O) CH₃, —C(O) CF₃, —C(O) CH₂OCH₃, and the like).

Similar to the substituents described for the alkyl radical, substituents for the aryl and heteroaryl groups are varied and are selected from, for example:—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″, —NR—C(NR′R″)═NR″, —S(O) R′, —S(O)₂R′, —S(O)₂NR′R″, —NRSO₂R′, —NR′NR″R′″, —ONR′R″, —NR′C(O)NR″NR″R′″, —CN, —NO₂, —R′, —N₃, —CH(Ph)₂, fluoro (C₁-C₄)alkoxy, and fluoro (C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings, bridged rings, or spirocyclic rings, a substituent depicted as associated with one member of the fused rings, bridged rings, or spirocyclic rings (a floating substituent on a single ring), may be a substituent on any of the fused rings, bridged rings, or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different bridged rings, or different spirocyclic rings, and each substituent may optionally be different. Where a point of attachment of a ring to the remainder of a molecule is not limited to a single atom (a floating substituent), the attachment point may be any atom of the ring and in the case of fused rings, bridged rings, or spirocyclic rings, any atom of any of the fused rings, bridged rings, or spirocyclic rings while obeying the rules of chemical valency. Where a ring, fused rings, bridged rings, or spirocyclic rings contain one or more ring heteroatoms and the ring, fused rings, bridged rings, or spirocyclic rings are shown with one or more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure and form a bridged ring structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

The terms “heteroatom” or “ring heteroatom” are meant to include oxygen (O), nitrogen (N), sulfur(S), phosphorus (P), and silicon (Si).

A “substituent group,” as used herein, means a group selected from the following moieties: oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (B) alkyl, heteroalkyl, cycloalkyl, heterocycloalkyl, aryl, heteroaryl, substituted with at least one substituent selected from: (i) oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C₁-C₈alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C5-C6 cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (ii) alkyl, heteroalkyl, cycloalkyl, heterocycloalkyl, aryl, heteroaryl, substituted with at least one substituent selected from: oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C1-C8 alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C5-C6 cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (b) alkyl, heteroalkyl, cycloalkyl, heterocycloalkyl, aryl, heteroaryl, substituted with at least one substituent selected from: oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C₁-C₈alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C5-C6 cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C6-C10 aryl, Co aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C6-C10 aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, each substituted group described in the compounds herein is substituted with at least one substituent group. In embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In embodiments, at least one or all of these groups are substituted with at least one size-limited substituent group. In embodiments, at least one or all of these groups are substituted with at least one lower substituent group.

In embodiments, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In aspects of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene.

Certain compounds may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed herein. The compounds do not include those that are known in art to be too unstable to synthesize and/or isolate. The disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the compounds are within the scope of the disclosure. The term “isomers” refers to compounds having the same number and kind of atoms, and hence the same molecular weight, but differing in respect to the structural arrangement or configuration of the atoms. The term “tautomer” refers to one of two or more structural isomers which exist in equilibrium and which are readily converted from one isomeric form to another. It will be apparent to one skilled in the art that certain compounds may exist in tautomeric forms, all such tautomeric forms of the compounds being within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure. The compounds may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds, whether radioactive or not, are encompassed herein.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a [n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹ substituent may be distinguished as R^{1A}, R^{1B}, etc., wherein each of R^{1A}, R^{1B}, etc. is defined within the scope of the definition of R¹ and optionally differently.

A “covalent cysteine modifier moiety” as used herein refers to a substituent that is capable of reacting with the sulfhydryl functional group of a cysteine amino acid (e.g. cysteine 442 of the interleukin-2-inducible T-cell kinase (ITK, TSK) (e.g., human interleukin-2-inducible T-cell kinase (ITK, TSK)), or amino acid corresponding to cysteine 442 of the interleukin-2-inducible T-cell kinase) to form a covalent bond. Thus, the covalent cysteine modifier moiety is typically electrophilic.

The term “electrophilic chemical moiety” is used in accordance with its plain ordinary chemical meaning and refers to a monovalent chemical group that is electrophilic.

Descriptions of compounds are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). In embodiments, the ITK inhibitors described herein are not in the form of a pharmaceutically acceptable salt.

Thus, the compounds may exist as salts, such as with pharmaceutically acceptable acids. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents. Certain compounds can exist in unsolvated forms as well as solvated forms, including hydrated forms. In general, the solvated forms are equivalent to unsolvated forms and are encompassed herein.

### ITK Inhibitors

An “interleukin-2-inducible T-cell kinase inhibitor” and “ITK inhibitor” is a compound that negatively affects (e.g. decreases) the activity or function of interleukin-2-inducible T-cell kinase relative to the activity or function of interleukin-2-inducible T-cell kinase in the absence of the inhibitor (e.g., wherein the ITK inhibitor binds ITK).

### ITK Selectivity Over RLK or BTK.

In embodiments, the ITK inhibitor is a compound having a selectivity for ITK that is at least 50-fold greater than the selectivity for resting lymphocyte kinase (RLK). In embodiments, the ITK inhibitor is a compound having a selectivity for ITK that is at least 60-fold greater than the selectivity for RLK. In embodiments, the ITK inhibitor is a compound having a selectivity for ITK that is at least 70-fold greater than the selectivity for RLK. In embodiments, the ITK inhibitor is a compound having a selectivity for ITK that is at least 80-fold greater than the selectivity for RLK. In embodiments, the ITK inhibitor is a compound having a selectivity for ITK that is at least 90-fold greater than the selectivity for RLK. In embodiments, the ITK inhibitor is a compound having a selectivity for ITK that is at least 100-fold greater than the selectivity for RLK. In embodiments, the ITK inhibitor is a compound having a selectivity for ITK that is at least 110-fold greater than the selectivity for RLK.

In embodiments, the ITK inhibitor is a compound having a selectivity for ITK that is at least 50-fold greater than the selectivity for Bruton's tyrosine kinase (BTK). In embodiments, the ITK inhibitor is a compound having a selectivity for ITK that is at least 60-fold greater than the selectivity for BTK. In embodiments, the ITK inhibitor is a compound having a selectivity for ITK that is at least 70-fold greater than the selectivity for BTK. In embodiments, the ITK inhibitor is a compound having a selectivity for ITK that is at least 80-fold greater than the selectivity for BTK. In embodiments, the ITK inhibitor is a compound having a selectivity for ITK that is at least 90-fold greater than the selectivity for BTK. In embodiments, the ITK inhibitor is a compound having a selectivity for ITK that is at least 100-fold greater than the selectivity for BTK.

In embodiments, the ITK inhibitor is a compound described in U.S. Pat. No. 11,008,314. In embodiments, the ITK inhibitor is a compound of Formula (I) or a pharmaceutically acceptable salt thereof. In embodiments, the ITK inhibitor is a compound of Formula (II) or a pharmaceutically acceptable salt thereof. In embodiments, the ITK inhibitor is a compound of Formula (A) or a pharmaceutically acceptable salt thereof. In embodiments, the ITK inhibitor is the free base form of the compound of Formula (I). In embodiments, the ITK inhibitor is the free base form of the compound of Formula (II). In embodiments, the ITK inhibitor is the free base form of the compound of Formula (A). In embodiments, the ITK inhibitor is a compound of Formula (1)-(34) or a pharmaceutically acceptable salt thereof. In embodiments, the ITK inhibitor is the free base form of a compound of Formula (1)-(34).

In embodiments, the ITK inhibitor is a compound of Formula (I) or a pharmaceutically acceptable salt thereof:

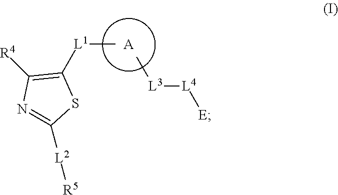

wherein the substitutents are as defined herein. In embodiments, the ITK inhibitor is any ITK inhibitor described in U.S. Pat. No. 11,008,314, the disclosure of which is incorporated by reference herein in its entirety and for all purposes.

### Ring A is

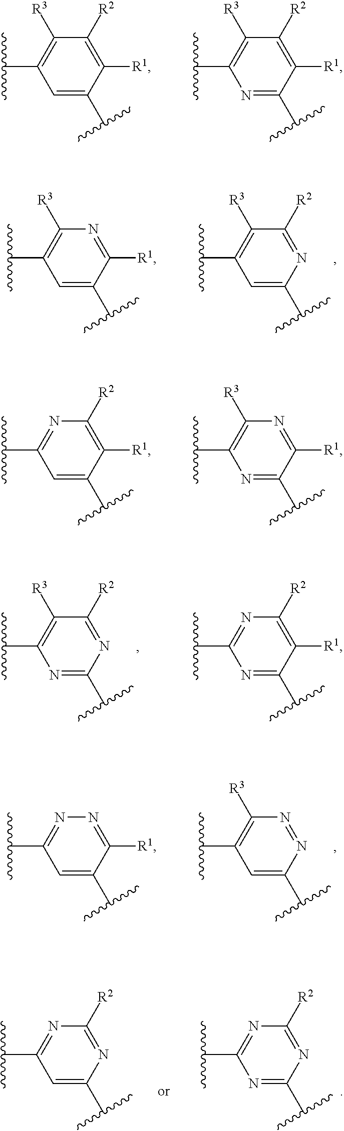

In aspects, Ring A is

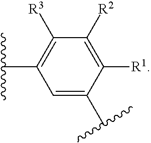

In embodiments, the compound of Formula (I) is a compound of Formula (II) or a pharmaceutically acceptable salt thereof:

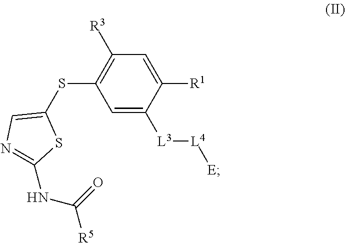

wherein the substitutents are as defined herein.

R¹, R², R³, and R⁴ are each independently hydrogen, halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ and R³ are each independently hydrogen, halogen —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, or —NR^{1A}OR^{1B}.

In aspects, one or both of R¹ and R³ are —OH. In aspects, one or both of R¹ and R³ are —NH₂. In aspects, one or both of R¹ and R³ are —COOH. In aspects, one or both of R¹ and R³ are —CONH₂. In aspects, one or both of R¹ and R³ are —NO₂. In aspects, one or both of R¹ and R³ are —SH. In aspects, one or both of R¹ and R³ are —CF₃. In aspects, one or both of R¹ and R³ are —CHF₂. In aspects, one or both of R¹ and R³ are —CH₂F. In aspects, one or both of R¹ and R³ are —OCF₃. In aspects, one or both of R¹ and R³ are —OCH₂F. In aspects, one or both of R¹ and R³ are —OCHF₂. In aspects, one or both of R¹ and R³ are —OCH₃. In aspects, one or both of R¹ and R³ are —OCH₂CH₃. In aspects, one or both of R¹ and R³ are —OCH₂CH₂CH₃. In aspects, one or both of R¹ and R³ are —OCH(CH₃)₂. In aspects, one or both of R¹ and R³ are —OC(CH₃)₃. In aspects, one or both of R¹ and R³ are —SCH₃. In aspects, one or both of R¹ and R³ are —SCH₂CH₃. In aspects, one or both of R¹ and R³ are —SCH₂CH₂CH₃. In aspects, one or both of R¹ and R³ are —SCH(CH₃)₂. In aspects, one or both of R¹ and R³ are —SC(CH₃)₃. In aspects, one or both of R¹ and R³ are —CH₃. In aspects, one or both of R¹ and R³ are —CH₂CH₃. In aspects, one or both of R¹ and R³ are —CH₂CH₂CH₃. In aspects, one or both of R¹ and R³ are —CH(CH₃)₂. In aspects, one or both of R¹ and R³ are —C(CH₃)₃. In aspects, one or both of R¹ and R³ are —F. In aspects, one or both of R¹ and R³ are —Cl. In aspects, one or both of R¹ and R³ are —Br. In aspects, one or both of R¹ and R³ are —I. In aspects, one or both of R¹ and R³ are hydrogen, methyl, ethyl, propyl, —CN, —COOH, —CONH₂, —F, —Cl, —Br, or —I.

R⁵ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In aspects, R⁵ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In aspects, R⁵ is substituted or unsubstituted (C₁-C₈) alkyl, substituted or unsubstituted 2 to 8 membered heteroalkyl, substituted or unsubstituted (C₃-C₆) cycloalkyl, substituted or unsubstituted 3 to 6 membered heterocycloalkyl, substituted or unsubstituted phenyl, or substituted or unsubstituted 5 to 6 membered heteroaryl. In aspects, R⁵ is substituted or unsubstituted (C₁-C₄) alkyl. In aspects, R⁵ is unsubstituted (C₁-C₄) alkyl. In aspects, R⁵ is unsubstituted methyl, unsubstituted ethyl, unsubstituted isopropyl, or unsubstituted tert-butyl. In aspects, R⁵ is substituted or unsubstituted 2 to 8 membered heteroalkyl. In aspects, R⁵ is substituted or unsubstituted 2 to 4 membered heteroalkyl. In aspects, R⁵ is unsubstituted 2 to 4 membered heteroalkyl. In aspects, R⁵ is-CH₂N(CH₃) 2. In aspects, R⁵ is substituted or unsubstituted (C₃-C₆) cycloalkyl. In aspects, R⁵ is unsubstituted (C₃-C₆) cycloalkyl. In aspects, R⁵ is unsubstituted cyclopropyl, unsubstituted cyclobutyl, or unsubstituted cyclopentyl. In aspects, R⁵ is substituted or unsubstituted 3 to 6 membered heterocycloalkyl. In aspects, R⁵ is substituted or unsubstituted 5 to 6 membered heterocycloalkyl. In aspects, R⁵ is substituted or unsubstituted 6 membered heterocycloalkyl. In aspects, R⁵ is substituted or unsubstituted piperidinyl. In aspects, R⁵ is substituted or unsubstituted phenyl. In aspects, R⁵ is unsubstituted phenyl. In aspects, R⁵ is 2-substituted phenyl. In aspects, R⁵ is 3-substituted phenyl. In aspects, R⁵ is 4-substituted phenyl. In aspects, R⁵ is phenyl substituted with halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In aspects, R⁵ is phenyl substituted with halogen, substituted or unsubstituted (C₁-C₅) alkyl, substituted or unsubstituted 2 to 8 membered heteroalkyl, substituted or unsubstituted (C₃-C₆) cycloalkyl, substituted or unsubstituted 3 to 6 membered heterocycloalkyl, substituted or unsubstituted phenyl, or substituted or unsubstituted 5 to 6 membered heteroaryl. In aspects, R⁵ is —N(CH₃)₂. In aspects, R⁵ is —NH(CH₃). In aspects, R⁵ is —N(CH₂CH₃)₂. In aspects, R⁵ is —NH(CH₂CH₃). In aspects, R⁵ is —N(CH₃)(CH₂CH₃). In aspects, R⁵ is —CH₃. In aspects, R⁵ is —CH₂CH₃. In aspects, R⁵ is unsubstituted isopropyl. In aspects, R⁵ is unsubstituted tert-butyl.

In aspects, R⁵ is substituted or unsubstituted heteroaryl. In aspects, R⁵ is substituted or unsubstituted 5 to 6 membered heteroaryl. In aspects, R⁵ is substituted or unsubstituted pyridyl, substituted or unsubstituted thienyl, substituted or unsubstituted furanyl, substituted or unsubstituted pyrrolyl, substituted or unsubstituted imidazolyl, substituted or unsubstituted pyrazolyl, substituted or unsubstituted thiazolyl, substituted or unsubstituted isothiazolyl, substituted or unsubstituted oxazolyl, or substituted or unsubstituted isoxazolyl.

In aspects, R⁵ is substituted or unsubstituted cycloalkyl (e.g., C₃-C₈ cycloalkyl, C₃-C₆ cycloalkyl, or C₅-C₆ cycloalkyl). In aspects, R⁵ is substituted or unsubstituted C₃-C₈ cycloalkyl. In aspects, R⁵ is substituted or unsubstituted C₃-C₆ cycloalkyl. In aspects, R⁵ is substituted or unsubstituted C₅-C₆ cycloalkyl. In aspects, R⁵ is substituted or unsubstituted C6 cycloalkyl. In aspects, R⁵ is substituted or unsubstituted C₅ cycloalkyl. In aspects, R⁵ is a substituted cycloalkyl (e.g., C₃-C₈ cycloalkyl, C₃-C₆ cycloalkyl, or C₅-C₆ cycloalkyl). In aspects, R⁵ is a substituted C₃-C₈ cycloalkyl. In aspects, R⁵ is a substituted C₃-C₆ cycloalkyl. In aspects, R⁵ is a substituted C₅-C₆ cycloalkyl. In aspects, R⁵ is a substituted C₆ cycloalkyl. In aspects, R⁵ is a substituted C₅ cycloalkyl. In aspects, R⁵ is an unsubstituted cycloalkyl (e.g., C₃-C₉ cycloalkyl, C₃-C₆ cycloalkyl, or C₅-C₆ cycloalkyl). In aspects, R⁵ is an unsubstituted C₃-C₅ cycloalkyl. In aspects, R⁵ is an unsubstituted C₃-C₆ cycloalkyl. In aspects, R⁵ is an unsubstituted C₅-C₆ cycloalkyl. In aspects, R⁵ is an unsubstituted C₆ cycloalkyl. In aspects, R⁵ is an unsubstituted C₅ cycloalkyl.

In aspects, R⁵ is substituted or unsubstituted aziridinyl, substituted or unsubstituted oziranyl, substituted or unsubstituted thiiranyl, substituted or unsubstituted azetidinyl, substituted or unsubstituted 1,2-dihydroazotyl, substituted or unsubstituted oxetanyl, substituted or unsubstituted 2H-oxetyl, substituted or unsubstituted thietanyl, substituted or unsubstituted 2H-thietyl, substituted or unsubstituted pyrrolidinyl, substituted or unsubstituted 2,5-dihydro-1H-pyrrolyl, substituted or unsubstituted 4,5-dihydro-1H-imidazolyl, substituted or unsubstituted imidazolinyl, substituted or unsubstituted pyrazolinyl, substituted or unsubstituted tetrahydrofuranyl, substituted or unsubstituted thiolanyl, substituted or unsubstituted piperidinyl, substituted or unsubstituted piperazinyl, substituted or unsubstituted 2H-pyranyl, substituted or unsubstituted morpholinyl, substituted or unsubstituted 1,4-dioxanyl, tetrahydro-2H-pyranyl, substituted or unsubstituted thianyl, or substituted or unsubstituted dithianyl. In aspects, R⁵ is a substituted aziridinyl, a substituted oziranyl, a substituted thiiranyl, a substituted azetidinyl, a substituted 1,2-dihydroazotyl, a substituted oxetanyl, a substituted 2H-oxetyl, a substituted thietanyl, a substituted 2H-thietyl, a substituted pyrrolidinyl, a substituted 2,5-dihydro-TH-pyrrolyl, a substituted 4,5-dihydro-TH-imidazolyl, a substituted imidazolinyl, a substituted pyrazolinyl, a substituted tetrahydrofuranyl, a substituted thiolanyl, a substituted piperidinyl, a substituted piperazinyl, a substituted 2H-pyranyl, a substituted morpholinyl, a substituted 1,4-dioxanyl, tetrahydro-2H-pyranyl, a substituted thianyl, or a substituted dithianyl. In aspects, R⁵ is an unsubstituted aziridinyl, an unsubstituted oziranyl, an unsubstituted thiiranyl, an unsubstituted azetidinyl, an unsubstituted 1,2-dihydroazotyl, an unsubstituted oxetanyl, an unsubstituted 2H-oxetyl, an unsubstituted thietanyl, an unsubstituted 2H-thietyl, an unsubstituted pyrrolidinyl, an unsubstituted 2,5-dihydro-TH-pyrrolyl, an unsubstituted 4,5-dihydro-1H-imidazolyl, an unsubstituted imidazolinyl, an unsubstituted pyrazolinyl, an unsubstituted tetrahydrofuranyl, an unsubstituted thiolanyl, an unsubstituted piperidinyl, an unsubstituted piperazinyl, an unsubstituted 2H-pyranyl, an unsubstituted morpholinyl, an unsubstituted 1,4-dioxanyl, tetrahydro-2H-pyranyl, an unsubstituted thianyl, or an unsubstituted dithianyl.

In aspects, R⁵ is substituted or unsubstituted (C₆-C₁₀) aryl. In aspects, R⁵ is substituted or unsubstituted phenyl. In aspects, R⁵ is substituted or unsubstituted naphthyl. In aspects, R⁵ is a substituted (C₆-C₁₀) aryl. In aspects, R⁵ is a substituted phenyl. In aspects, R⁵ is a substituted naphthyl. In aspects, R⁵ is an unsubstituted (C₆-C₁₀) aryl. In aspects, R⁵ is an unsubstituted phenyl. In aspects, R⁵ is an unsubstituted naphthyl.

In aspects, R⁵ is imidazolyl, substituted or unsubstituted pyrrolyl, substituted or unsubstituted pyrazolyl, substituted or unsubstituted triazolyl, substituted or unsubstituted tetrazolyl, substituted or unsubstituted furanyl, substituted or unsubstituted oxazolyl, substituted or unsubstituted isooxazolyl, substituted or unsubstituted oxadiazolyl, substituted or unsubstituted oxatriazolyl, substituted or unsubstituted thienyl, substituted or unsubstituted thiazolyl, substituted or unsubstituted isothiazolyl, substituted or unsubstituted pyridinyl, substituted or unsubstituted pyrazinyl, substituted or unsubstituted pyrimidinyl, substituted or unsubstituted pyridazinyl, or substituted or unsubstituted triazinyl (e.g., 1,3,5-triazinyl, 1,2,3-triazinyl, or 1,2,4-triazinyl). In aspects, R⁵ is imidazolyl, a substituted pyrrolyl, a substituted pyrazolyl, a substituted triazolyl, a substituted tetrazolyl, a substituted furanyl, a substituted oxazolyl, a substituted isooxazolyl, a substituted oxadiazolyl, a substituted oxatriazolyl, a substituted thienyl, a substituted thiazolyl, a substituted isothiazolyl, a substituted pyridinyl, a substituted pyrazinyl, a substituted pyrimidinyl, a substituted pyridazinyl, or a substituted triazinyl (e.g., 1,3,5-triazinyl, 1,2,3-triazinyl, or 1,2,4-triazinyl). In aspects, R⁵ is imidazolyl, an unsubstituted pyrrolyl, an unsubstituted pyrazolyl, an unsubstituted triazolyl, an unsubstituted tetrazolyl, an unsubstituted furanyl, an unsubstituted oxazolyl, an unsubstituted isooxazolyl, an unsubstituted oxadiazolyl, an unsubstituted oxatriazolyl, an unsubstituted thienyl, an unsubstituted thiazolyl, an unsubstituted isothiazolyl, an unsubstituted pyridinyl, an unsubstituted pyrazinyl, an unsubstituted pyrimidinyl, an unsubstituted pyridazinyl, or an unsubstituted triazinyl (e.g., 1,3,5-triazinyl, 1,2,3-triazinyl, or 1,2,4-triazinyl).

In aspects, R⁵ is

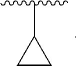 [SMILES: *C1CC1]

In aspects, R⁵ is

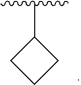 [SMILES: *C1CCC1]

In aspects, R⁵ is

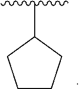 [SMILES: *C1CCCC1]

In aspects, R⁵ is substituted or unsubstituted alkyl (e.g., C₁-C₈, C₁-C₆, C₁-C₄, or C₁-C₂). In aspects, R⁵ is substituted alkyl (e.g., C₁-C₈, C₁-C₆, C₁-C₄, or C₁-C₂). In aspects, R⁵ is unsubstituted alkyl (e.g., C₁-C₈, C₁-C₆, C₁-C₄, or C₁-C₂). In aspects, R⁵ is unsubstituted methyl. In aspects, R⁵ is unsubstituted ethyl. In aspects, R⁵ is unsubstituted propyl. In aspects, R⁵ is unsubstituted isopropyl. In aspects, R⁵ is unsubstituted tert-butyl. In aspects, R⁵ is substituted or unsubstituted heteroalkyl (e.g., 2 to 8 membered, 2 to 6 membered, 4 to 6 membered, 2 to 3 membered, or 4 to 5 membered). In aspects, R⁵ is substituted heteroalkyl (e.g., 2 to 8 membered, 2 to 6 membered, 4 to 6 membered, 2 to 3 membered, or 4 to 5 membered). In aspects, R⁵ is unsubstituted heteroalkyl (e.g., 2 to 8 membered, 2 to 6 membered, 4 to 6 membered, 2 to 3 membered, or 4 to 5 membered). In aspects, R⁵ is substituted or unsubstituted cycloalkyl (e.g., C₃-C₈, C₃-C₆, C₄-C₆, or C₅-C₆). In aspects, R⁵ is substituted cycloalkyl (e.g., C₃-C₈, C₃-C₆, C₄-C₆, or C₅-C₆). In aspects, R⁵ is unsubstituted cycloalkyl (e.g., C₃-C₈, C₃-C₆, C₄-C₆, or C₅-C₆). In aspects, R⁵ is substituted or unsubstituted heterocycloalkyl (e.g., 3 to 8 membered, 3 to 6 membered, 4 to 6 membered, 4 to 5 membered, or 5 to 6 membered). In aspects, R⁵ is substituted heterocycloalkyl (e.g., 3 to 8 membered, 3 to 6 membered, 4 to 6 membered, 4 to 5 membered, or 5 to 6 membered). In aspects, R⁵ is unsubstituted heterocycloalkyl (e.g., 3 to 8 membered, 3 to 6 membered, 4 to 6 membered, 4 to 5 membered, or 5 to 6 membered). In aspects, R⁵ is substituted or unsubstituted aryl (e.g., C₆-C₁₀ or phenyl). In aspects, R⁵ is substituted aryl (e.g., C₆-C₁₀ or phenyl). In aspects, R⁵ is unsubstituted aryl (e.g., C₆-C₁₀ or phenyl). In aspects, R⁵ is substituted or unsubstituted heteroaryl (e.g., 5 to 10 membered, 5 to 9 membered, or 5 to 6 membered). In aspects, R⁵ is substituted heteroaryl (e.g., 5 to 10 membered, 5 to 9 membered, or 5 to 6 membered). In aspects, R⁵ is unsubstituted heteroaryl (e.g., 5 to 10 membered, 5 to 9 membered, or 5 to 6 membered).

In aspects, R⁵ is substituted or unsubstituted pyrrolidinyl. In aspects, R⁵ is substituted or unsubstituted tetrahydrofuranyl. In aspects, R⁵ is substituted or unsubstituted imidazolidinyl. In aspects, R⁵ is substituted or unsubstituted pyrazolidinyl. In aspects, R⁵ is substituted or unsubstituted oxazolidinyl. In aspects, R⁵ is substituted or unsubstituted isoxazolidinyl. In aspects, R⁵ is substituted or unsubstituted thiazolidinyl. In aspects, R⁵ is substituted or unsubstituted isothiazolidinyl. In aspects, R⁵ is substituted or unsubstituted dioxolanyl. In aspects, R⁵ is substituted or unsubstituted dithiolanyl. In aspects, R⁵ is substituted or unsubstituted piperidinyl. In aspects, R⁵ is substituted or unsubstituted oxanyl. In aspects, R⁵ is substituted or unsubstituted piperazinyl. In aspects, R⁵ is substituted or unsubstituted morpholinyl. In aspects, R⁵ is substituted or unsubstituted pyridinyl. In aspects, R⁵ is substituted or unsubstituted triazolyl. In aspects, R⁵ is substituted or unsubstituted tetrazolyl. In aspects, R⁵ is substituted or unsubstituted benzo[d][1,3]dioxolyl. In aspects, R⁵ is substituted or unsubstituted phenyl. In aspects, R⁵ is substituted or unsubstituted pyridyl. In aspects, R⁵ is substituted or unsubstituted pyridazinyl. In aspects, R⁵ is substituted or unsubstituted pyrimidinyl. In aspects, R⁵ is substituted or unsubstituted pyrazinyl. In aspects, R⁵ is substituted or unsubstituted piperidinyl. In aspects, R⁵ is substituted or unsubstituted tetrahydropyranyl. In aspects, R⁵ is substituted or unsubstituted tetrahydrothiopyranyl. In aspects, R⁵ is substituted or unsubstituted cyclohexyl. In aspects, R⁵ is substituted or unsubstituted cyclopentyl. In aspects, R⁵ is substituted or unsubstituted cycloheptyl. In aspects, R⁵ is substituted or unsubstituted cyclobutyl. In aspects, R⁵ is substituted or unsubstituted cyclopropyl. In aspects, R⁵ is substituted or unsubstituted pyrrolyl. In aspects, R⁵ is substituted or unsubstituted furanyl. In aspects, R⁵ is substituted or unsubstituted thienyl. In aspects, R⁵ is substituted or unsubstituted pyrazolyl. In aspects, R⁵ is substituted or unsubstituted imidazolyl. In aspects, R⁵ is substituted or unsubstituted isoxazolyl. In aspects, R⁵ is substituted or unsubstituted oxazolyl. In aspects, R⁵ is substituted or unsubstituted isothiazolyl. In aspects, R⁵ is substituted or unsubstituted thiazolyl. In aspects, R⁵ is substituted or unsubstituted naphthyl. In aspects, R⁵ is substituted or unsubstituted quinolinyl. In aspects, R⁵ is substituted or unsubstituted isoquinolinyl. In aspects, R⁵ is substituted or unsubstituted indolyl. In aspects, R⁵ is substituted or unsubstituted benzimidazolyl. In aspects, R⁵ is substituted or unsubstituted indazolyl. In aspects, R⁵ is substituted or unsubstituted isoindolyl. In aspects, R⁵ is substituted or unsubstituted benzofuranyl. In aspects, R⁵ is substituted or unsubstituted benzo[c]thienyl. In aspects, R⁵ is substituted or unsubstituted 2,3-dihydro-TH-indenyl. In aspects, R⁵ is substituted or unsubstituted 1,2,3,4-tetrahydronaphthyl. In aspects, R⁵ is substituted or unsubstituted triazolyl. In aspects, R⁵ is substituted or unsubstituted quinoxalinyl. In aspects, R⁵ is substituted or unsubstituted quinazolinyl. In aspects, R⁵ is substituted or unsubstituted triazinyl. In aspects, R⁵ is substituted or unsubstituted cinnolinyl. In aspects, R⁵ is substituted or unsubstituted phthalazinyl. In aspects, R⁵ is substituted or unsubstituted benzoxazolyl. In aspects, R⁵ is substituted or unsubstituted benzisoxazolyl. In aspects, R⁵ is substituted or unsubstituted benzothiazolyl. In aspects, R⁵ is substituted or unsubstituted benzisothiazolyl. In aspects, R⁵ is substituted or unsubstituted benzo[d][1,2,3]triazolyl. In aspects, R⁵ is substituted or unsubstituted adamantyl.

L¹ is —O—, —S—, or substituted or unsubstituted C₁-C₂ alkylene, or substituted or unsubstituted 2 membered heteroalkylene. In aspect, L¹ is —O—. In aspect, L¹ is —S—. In aspect, L¹ is substituted C₁-C₂ alkylene. In aspect, L¹ is unsubstituted C₁-C₂ alkylene. In aspect, L¹ is substituted 2 membered heteroalkylene. In aspect, L¹ is unsubstituted 2 membered heteroalkylene.

L² is a bond, —NH—, —C(O)NH—, or —NHC(O)—. In aspects, L² is a bond. In aspects, L² is —NH—. In aspects, L² is —NHC(O)—.

L³ and L⁴ are each independently a bond, —S(O)₂—, —N(R⁶)—, —O—, —S—, —C(O)—, —C(O)N(R⁶)—, —N(R⁶)C(O)—, —N(R⁶)C(O)NH—, —NHC(O)N(R⁶)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In aspects, L³ is a bond, —N(R⁶)—, —C(O)—, —C(O)N(R⁶)—, —N(R⁶)C(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenyl), or substituted or unsubstituted heteroarylene (e.g., 5 to 10 membered, 5 to 9 membered, or 5 to 6 membered).

In aspects, L³ is a bond, —N(R⁶)—, —C(O)—, or —C(O)N(R⁶)—; and R⁶ is hydrogen, —CX⁶₃, —CHX⁶₂, —CH₂X⁶, or unsubstituted (C₁-C₄) alkyl. In aspects, L³ is a bond, —N(R⁶)—, —C(O)—, or —C(O)N(R⁶)—; and R⁶ is hydrogen or unsubstituted methyl. In aspects, L³ is a bond, —C(O)—, —C(O)N(CH₃)—, —N(CH₃)—, or —NH—. In aspects, L³ is a bond. In aspects, L³ is —C(O)—. In aspects, L³ is —N(R⁶)—. In aspects, L³ is —C(O)—. In aspects, L³ is —C(O)N(R⁶)—. In aspects, L³ is —NH—. In aspects, L³ is —C(O)—. In aspects, L³ is —C(O)NH—. In aspects, L³ is —N(CH₃)—. In aspects, L³ is —C(O)N(CH₃)—. In aspects, L³ is —N(CH₂CH₃)—. In aspects, L³ is —C(O)N(CH₂CH₃)—.

In aspects, L³ is substituted or unsubstituted alkylene (e.g., C₁-C₈, C₁-C₆, C₁-C₄, or C₁-C₂). In aspects, L³ is substituted alkylene (e.g., C₁-C₈, C₁-C₆, C₁-C₄, or C₁-C₂). In aspects, L³ is unsubstituted alkylene (e.g., C₁-C₈, C₁-C₆, C₁-C₄, or C₁-C₂). In aspects, L³ is unsubstituted methylene. In aspects, L³ is unsubstituted ethylene. In aspects, L³ is unsubstituted propylene. In aspects, L³ is unsubstituted isopropylene. In aspects, L³ is unsubstituted tert-butylene. In aspects, L³ is substituted or unsubstituted heteroalkylene (e.g., 2 to 8 membered, 2 to 6 membered, 4 to 6 membered, 2 to 3 membered, or 4 to 5 membered). In aspects, L³ is substituted heteroalkylene (e.g., 2 to 8 membered, 2 to 6 membered, 4 to 6 membered, 2 to 3 membered, or 4 to 5 membered). In aspects, L³ is unsubstituted heteroalkylene (e.g., 2 to 8 membered, 2 to 6 membered, 4 to 6 membered, 2 to 3 membered, or 4 to 5 membered). In aspects, L³ is substituted or unsubstituted cycloalkylene (e.g., C₃-C₈, C₃-C₆, C₄-C₆, or C₅-C₆). In aspects, L³ is substituted cycloalkylene (e.g., C₃-C₈, C₃-C₆, C₄-C₆, or C₅-C₆). In aspects, L³ is unsubstituted cycloalkylene (e.g., C₃-C₈, C₃-C₆, C₄-C₆, or C₅-C₆). In aspects, L³ is substituted or unsubstituted heterocycloalkylene (e.g., 3 to 8 membered, 3 to 6 membered, 4 to 6 membered, 4 to 5 membered, or 5 to 6 membered). In aspects, L³ is substituted heterocycloalkylene (e.g., 3 to 8 membered, 3 to 6 membered, 4 to 6 membered, 4 to 5 membered, or 5 to 6 membered). In aspects, L³ is unsubstituted heterocycloalkylene (e.g., 3 to 8 membered, 3 to 6 membered, 4 to 6 membered, 4 to 5 membered, or 5 to 6 membered). In aspects, L³ is substituted or unsubstituted arylene (e.g., C₆-C₁₀ or phenylene). In aspects, L³ is substituted arylene (e.g., C₆-C₁₀ or phenylene). In aspects, L³ is unsubstituted arylene (e.g., C₆-C₁₀ or phenylene). In aspects, L³ is substituted or unsubstituted heteroarylene (e.g., 5 to 10 membered, 5 to 9 membered, or 5 to 6 membered). In aspects, L³ is substituted heteroarylene (e.g., 5 to 10 membered, 5 to 9 membered, or 5 to 6 membered). In aspects, L³ is unsubstituted heteroarylene (e.g., 5 to 10 membered, 5 to 9 membered, or 5 to 6 membered).

In aspects, L³ is a bond, —S(O)₂—, —N(R⁶)—, —O—, —S—, —C(O)—, —C(O)N(R⁶)—, —N(R⁶)C(O)—, —N(R⁶)C(O)NH—, —NHC(O)N(R⁶)—, —C(O)O—, —OC(O)—, a substituted or unsubstituted alkylene (e.g., C₁-C₈, C₁-C₆, C₁-C₄, or C₁-C₂), a substituted or unsubstituted heteroalkylene (e.g., 2 to 8 membered, 2 to 6 membered, 4 to 6 membered, 2 to 3 membered, or 4 to 5 membered), a substituted or unsubstituted cycloalkylene (e.g., C₃-C₈, C₃-C₆, C4-C6, or C5-C6), a substituted or unsubstituted heterocycloalkylene (e.g., 3 to 8 membered, 3 to 6 membered, 4 to 6 membered, 4 to 5 membered, or 5 to 6 membered), a substituted or unsubstituted arylene (e.g., C₆-C₁₀ or phenylene), or a substituted or unsubstituted heteroarylene (e.g., 5 to 10 membered, 5 to 9 membered, or 5 to 6 membered). In aspects, L³ is a bond, —S(O)₂—, —N(R⁶)—, —O—, —S—, —C(O)—, —C(O)N(R⁶)—, —N(R⁶)C(O)—, —N(R⁶)C(O)NH—, —NHC(O)N(R⁶)—, —C(O)O—, —OC(O)—, unsubstituted alkylene (e.g., C₁-C₈, C₁-C₆, C₁-C₄, or C₁-C₂), unsubstituted heteroalkylene (e.g., 2 to 8 membered, 2 to 6 membered, 4 to 6 membered, 2 to 3 membered, or 4 to 5 membered), unsubstituted cycloalkylene (e.g., C₃-C₈, C₃-C₆, C₄-C₆, or C₅-C₆), unsubstituted heterocycloalkylene (e.g., 3 to 8 membered, 3 to 6 membered, 4 to 6 membered, 4 to 5 membered, or 5 to 6 membered), unsubstituted arylene (e.g., C₆-C₁₀ or phenylene), or unsubstituted heteroarylene (e.g., 5 to 10 membered, 5 to 9 membered, or 5 to 6 membered). In aspects, L³ is unsubstituted methylene. In aspects, L³ is unsubstituted ethylene. In aspects, L³ is methyl-substituted methylene.

In aspects, L⁴ is a bond, —N(R⁶)—, —C(O)—, —C(O)N(R⁶)—, —N(R⁶)C(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenyl), or substituted or unsubstituted heteroarylene (e.g., 5 to 10 membered, 5 to 9 membered, or 5 to 6 membered).

In aspects, L⁴ is a bond. In aspects, L⁴ is —N(R⁶)—. In aspects, L⁴ is —C(O)N(R⁶)—. In aspects, L⁴ is —NH—. In aspects, L⁴ is —C(O)—. In aspects, L⁴ is —C(O)NH—. In aspects, L⁴ is —N(CH₃)—. In aspects, L⁴ is —C(O)N(CH₃)—. In aspects, L⁴ is —N(CH₂CH₃)—. In aspects, L⁴ is —C(O)N(CH₂CH₃)—. In aspects, L⁴ is a bond, —N(R⁷)—, —C(O)—, —C(O)N(R⁷)—, substituted or unsubstituted heterocycloalkylene, substituted or unsubstituted fused ring heterocycloalkylene, substituted or unsubstituted spirocyclic heterocycloalkylene, or substituted or unsubstituted bridged ring heterocycloalkylene; and R⁶ is hydrogen, —CX⁶₃, —CHX⁶₂, —CH₂X⁶, or unsubstituted (C₁-C₄) alkyl.

In aspects, L⁴ is a bond, substituted or unsubstituted monocyclic heterocycloalkylene, substituted or unsubstituted fused ring heterocycloalkylene, substituted or unsubstituted spirocyclic heterocycloalkylene, or substituted or unsubstituted bridged ring heterocycloalkylene. In aspects, L⁴ is unsubstituted 7 to 8 membered bridged ring heterocycloalkylene. In aspects, L⁴ is unsubstituted 7 to 8 membered fused ring heterocycloalkylene. In aspects, L⁴ is unsubstituted 7 to 8 membered spirocyclic heterocycloalkylene. In aspects, L⁴ is unsubstituted 5 to 8 membered monocyclic heterocycloalkylene. In aspects, L⁴ is methyl-substituted 5 to 8 membered monocyclic heterocycloalkylene. In aspects, L⁴ is methyl-substituted 6 to 7 membered monocyclic heterocycloalkylene. In aspects, L⁴ is ethyl-substituted 5 to 8 membered monocyclic heterocycloalkylene. In aspects, L⁴ is ethyl-substituted 6 to 7 membered monocyclic heterocycloalkylene. In aspects, L⁴ is cyano-substituted 5 to 8 membered monocyclic heterocycloalkylene. In aspects, L⁴ is cyano-substituted 6 to 7 membered monocyclic heterocycloalkylene. In aspects, L⁴ is halo-substituted 5 to 8 membered monocyclic heterocycloalkylene. In aspects, L⁴ is halo-substituted 6 to 7 membered monocyclic heterocycloalkylene. In aspects, L⁴ is unsubstituted 5 to 8 membered monocyclic heterocycloalkylene. In aspects, L⁴ is unsubstituted 6 to 7 membered monocyclic heterocycloalkylene. In aspects, L⁴ is ethyl-substituted 5 to 8 membered monocyclic heterocycloalkylene. In aspects, L⁴ is ethyl-substituted 6 to 7 membered monocyclic heterocycloalkylene. In aspects, L⁴ is isopropyl-substituted 5 to 8 membered monocyclic heterocycloalkylene. In aspects, L⁴ is isopropyl-substituted 6 to 7 membered monocyclic heterocycloalkylene. In aspects, L⁴ is tert-butyl-substituted 5 to 8 membered monocyclic heterocycloalkylene. In aspects, L⁴ is a tert-butyl-substituted 6 to 7 membered monocyclic heterocycloalkylene. In aspects, L⁴ is substituted or unsubstituted 4 to 10 membered monocyclic heterocycloalkylene. In aspects, L⁴ is substituted or unsubstituted 5 to 8 membered monocyclic heterocycloalkylene. In aspects, L⁴ is substituted 5 to 8 membered monocyclic heterocycloalkylene. In aspects, L⁴ is unsubstituted 5 to 8 membered monocyclic heterocycloalkylene. In aspects, L⁴ is substituted or unsubstituted 5 to 10 membered fused ring heterocycloalkylene. In aspects, L⁴ is substituted or unsubstituted 7 to 8 membered fused ring heterocycloalkylene. In aspects, L⁴ is substituted 5 to 10 membered fused ring heterocycloalkylene. In aspects, L⁴ is substituted 7 to 8 membered fused ring heterocycloalkylene. In aspects, L⁴ is unsubstituted 5 to 10 membered fused ring heterocycloalkylene. In aspects, L⁴ is unsubstituted 7 to 8 membered fused ring heterocycloalkylene. In aspects, L⁴ is unsubstituted 5 membered bridged ring heterocycloalkylene. In aspects, L⁴ is unsubstituted 6 membered bridged ring heterocycloalkylene. In aspects, L⁴ is unsubstituted 7 membered bridged ring heterocycloalkylene. In aspects, L⁴ is unsubstituted 8 membered bridged ring heterocycloalkylene. In aspects, L⁴ is unsubstituted 9 membered bridged ring heterocycloalkylene. In aspects, L⁴ is unsubstituted 10 membered bridged ring heterocycloalkylene. In aspects, L⁴ is unsubstituted 5 membered heterocycloalkylene. In aspects, L⁴ is unsubstituted 6 membered heterocycloalkylene. In aspects, L⁴ is unsubstituted 7 membered heterocycloalkylene. In aspects, L⁴ is unsubstituted 8 membered heterocycloalkylene. In aspects, L⁴ is unsubstituted 9 membered heterocycloalkylene. In aspects, L⁴ is unsubstituted 10 membered heterocycloalkylene.

In aspects, L⁴ is substituted or unsubstituted cycloalkylene. In aspects, L⁴ is substituted or unsubstituted C₃-C₈ cycloalkylene. In aspects, L⁴ is substituted or unsubstituted C₃-C₆ cycloalkylene. In aspects, L⁴ is substituted or unsubstituted C₅-C₆ cycloalkylene. In aspects, L⁴ is substituted or unsubstituted C₆ cycloalkylene. In aspects, L⁴ is substituted or unsubstituted C₅ cycloalkylene. In aspects, L⁴ is substituted cycloalkylene. In aspects, L⁴ is substituted C₃-C₈ cycloalkylene. In aspects, L⁴ is substituted C₃-C₆ cycloalkylene. In aspects, L⁴ is substituted C₅-C₆ cycloalkylene. In aspects, L⁴ is substituted C₆ cycloalkylene. In aspects, L⁴ is substituted C₅ cycloalkylene. In aspects, L⁴ is an unsubstituted cycloalkylene. In aspects, L⁴ is an unsubstituted C₃-C₈ cycloalkylene. In aspects, L⁴ is an unsubstituted C₃-C₆ cycloalkylene. In aspects, L⁴ is an unsubstituted C₅-C₆ cycloalkylene. In aspects, L⁴ is an unsubstituted C₆ cycloalkylene. In aspects, L⁴ is an unsubstituted C₅ cycloalkylene.

In aspects, L⁴ is substituted or unsubstituted aziridinylene, substituted or unsubstituted oziranylene, substituted or unsubstituted thiiranylene, substituted or unsubstituted azetidinylene, substituted or unsubstituted 1,2-dihydroazotylene, substituted or unsubstituted oxetanylene, substituted or unsubstituted 2H-oxetylene, substituted or unsubstituted thietanylene, substituted or unsubstituted 2H-thietylene, substituted or unsubstituted pyrrolidinylene, substituted or unsubstituted 2,5-dihydro-1H-pyrrolylene, substituted or unsubstituted 4,5-dihydro-TH-imidazolylene, substituted or unsubstituted imidazolinylene, substituted or unsubstituted pyrazolinylene, substituted or unsubstituted tetrahydrofuranylene, substituted or unsubstituted thiolanylene, substituted or unsubstituted piperidinylene, substituted or unsubstituted piperazinylene, substituted or unsubstituted 2H-pyranylene, substituted or unsubstituted morpholinylene, substituted or unsubstituted 1,4-dioxanylene, substituted or unsubstituted tetrahydro-2H-pyranylene, substituted or unsubstituted thianylene, or substituted or unsubstituted dithianylene. In aspects, L⁴ is substituted aziridinylene, substituted oziranylene, substituted thiiranylene, substituted azetidinylene, substituted 1,2-dihydroazotylene, substituted oxetanylene, substituted 2H-oxetylene, substituted thietanylene, substituted 2H-thietylene, substituted pyrrolidinylene, substituted 2,5-dihydro-1H-pyrrolylene, substituted 4,5-dihydro-1H-imidazolylene, substituted imidazolinylene, substituted pyrazolinylene, substituted tetrahydrofuranylene, substituted thiolanylene, substituted piperidinylene, substituted piperazinylene, substituted 2H-pyranylene, substituted morpholinylene, substituted 1,4-dioxanvlene, substituted tetrahydro-2H-pyranylene, substituted thianylene, or substituted dithianylene. In aspects, L⁴ is an unsubstituted aziridinylene, an unsubstituted oziranylene, an unsubstituted thiiranylene, an unsubstituted azetidinylene, an unsubstituted 1,2-dihydroazotylene, an unsubstituted oxetanylene, an unsubstituted 2H-oxetylene, an unsubstituted thietanvlene, an unsubstituted 2H-thietylene, an unsubstituted pyrrolidinylene, an unsubstituted 2,5-dihydro-1H-pyrrolylene, an unsubstituted 4,5-dihydro-1H-imidazolylene, an unsubstituted imidazolinylene, an unsubstituted pyrazolinylene, an unsubstituted tetrahydrofuranylene, an unsubstituted thiolanylene, an unsubstituted piperidinylene, an unsubstituted piperazinylene, an unsubstituted 2H-pyranylene, an unsubstituted morpholinylene, an unsubstituted 1,4-dioxanylene, an unsubstituted tetrahydro-2H-pyranylene, an unsubstituted thianylene, or an unsubstituted dithianylene.

In aspects, L⁴ is substituted or unsubstituted (C₆-C₁₀) arylene. In aspects, L⁴ is substituted or unsubstituted phenylene. In aspects, L⁴ is substituted or unsubstituted naphthylene. In aspects, L⁴ is substituted (C₆-C₁₀) arylene. In aspects, L⁴ is substituted phenylene. In aspects, L⁴ is substituted naphthylene. In aspects, L⁴ is an unsubstituted (C₆-C₁₀) arylene. In aspects. L⁴ is an unsubstituted phenylene. In aspects, L⁴ is an unsubstituted naphthylene.

In aspects, L⁴ is substituted or unsubstituted imidazolylene, substituted or unsubstituted pyrrolylene, substituted or unsubstituted pyrazolylene, substituted or unsubstituted triazolylene, substituted or unsubstituted tetrazolylene, substituted or unsubstituted furanylene, substituted or unsubstituted oxazolylene, substituted or unsubstituted isooxazolylene, substituted or unsubstituted oxadiazolylene, substituted or unsubstituted oxatriazolylene, substituted or unsubstituted thienylene, substituted or unsubstituted thiazolylene, substituted or unsubstituted isothiazolylene, substituted or unsubstituted pyridinylene, substituted or unsubstituted pyrazinylene, substituted or unsubstituted pyrimidinylene, substituted or unsubstituted pyridazinylene, substituted or unsubstituted triazinylene (e.g., 1,3,5-triazinylene, 1,2,3-triazinylene, or 1,2,4-triazinylene). In aspects, L⁴ is substituted imidazolylene, substituted pyrrolylene, substituted pyrazolylene, substituted triazolylene, substituted tetrazolylene, substituted furanylene, substituted oxazolylene, substituted isooxazolylene, substituted oxadiazolylene, substituted oxatriazolylene, substituted thienylene, substituted thiazolylene, substituted isothiazolylene, substituted pyridinylene, substituted pyrazinylene, substituted pyrimidinylene, substituted pyridazinylene, or substituted triazinylene (e.g., 1,3,5-triazinylene, 1,2,3-triazinylene, or 1,2,4-triazinylene). In aspects, L⁴ is an unsubstituted imidazolylene, an unsubstituted pyrrolylene, an unsubstituted pyrazolylene, an unsubstituted triazolylene, an unsubstituted tetrazolylene, an unsubstituted furanylene, an unsubstituted oxazolylene, an unsubstituted isooxazolylene, an unsubstituted oxadiazolylene, an unsubstituted oxatriazolylene, an unsubstituted thienylene, an unsubstituted thiazolylene, an unsubstituted isothiazolylene, an unsubstituted pyridinylene, an unsubstituted pyrazinylene, an unsubstituted pyrimidinylene, an unsubstituted pyridazinylene, or an unsubstituted triazinylene (e.g., 1,3,5-triazinylene, 1,2,3-triazinylene, or 1,2,4-triazinylene).

In aspects, L⁴ is

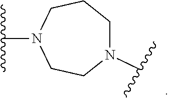 [SMILES: *N1CCCN(*)CC1]

In aspects, L⁴ is

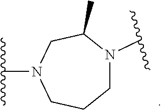 [SMILES: *N1CCCN(*)[C@H](C)C1]

In aspects, L⁴ is

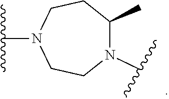 [SMILES: *N1CC[C@@H](C)N(*)CC1]

In aspects, L⁴ is

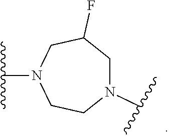 [SMILES: *N1CCN(*)CC(F)C1]

In aspects, L⁴ is

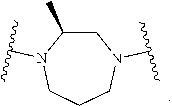 [SMILES: *N1CCCN(*)[C@@H](C)C1]

In aspects, L⁴ is

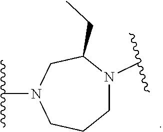 [SMILES: *N1CCCN(*)[C@H](CC)C1]

In aspects, L⁴ is

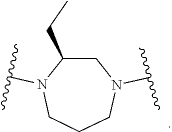 [SMILES: *N1CCCN(*)[C@@H](CC)C1]

In aspects, L⁴ is

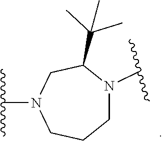 [SMILES: *N1CCCN(*)[C@H](C(C)(C)C)C1]

In aspects, L⁴ is

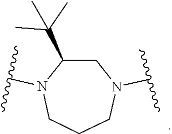 [SMILES: *N1CCCN(*)[C@@H](C(C)(C)C)C1]

In aspects, L⁴ is

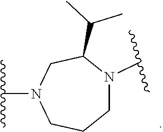 [SMILES: *N1CCCN(*)[C@H](C(C)C)C1]

In aspects, L⁴ is

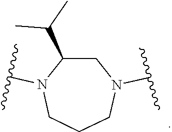 [SMILES: *N1CCCN(*)[C@@H](C(C)C)C1]

In aspects, L⁴ is

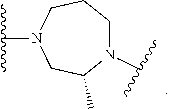 [SMILES: *N1CCCN(*)[C@H](C)C1]

In aspects, L⁴ is

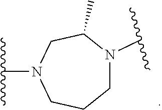 [SMILES: *N1CCCN(*)[C@@H](C)C1]

R⁶ is independently hydrogen, —CX⁶₃, —CHX⁶₂, —CH₂X⁶, —CN, —C(O)R^{6A}, —C(O)OR^{6A}, —C(O)NR^{6A}R^{6B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In aspects, R⁶ is independently hydrogen, —CX⁶₃, —CHX⁶₂, —CH₂X⁶, —CN, —C(O)R^{6A}, —C(O)—OR^{6A}, —C(O)NR^{6A}R^{6B}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In aspects, R⁶ is hydrogen. In aspects, R⁶ is —CX⁶₃. In aspects, R⁶ is —CHX⁶₂. In aspects, R⁶ is —CH₂X⁶. In aspects, R⁶ is —CN. In aspects, R⁶ is —C(O)R^{6A}. In aspects, R⁶ is —C(O)—OR^{6A} In aspects, R⁶ is —C(O)NR^{6A}R^{6B}. In aspects, R⁶ is —COOH. In aspects, R⁶ is —CONH₂. In aspects, R⁶ is —CF₃. In aspects, R⁶ is —CHF₂. In aspects, R⁶ is —CH₂F. In aspects, R⁶ is —CH₃. In aspects, R⁶ is —CH₂CH₃. In aspects, R⁶ is —CH₂CH₂CH₃. In aspects, R⁶ is —CH(CH₃)₂. In aspects, R⁶ is —C(CH₃)₃.

In aspects, R⁶ is substituted or unsubstituted alkyl (e.g., C₁-C₈, C₁-C₆, C₁-C₄, or C₁-C₂). In aspects, R⁶ is substituted alkyl (e.g., C₁-C₈, C₁-C₆, C₁-C₄, or C₁-C₂). In aspects, R⁶ is unsubstituted alkyl (e.g., C1-C₈, C₁-C₆, C₁-C₄, or C₁-C₂). In aspects, R⁶ is unsubstituted methyl. In aspects, R⁶ is unsubstituted ethyl. In aspects, R⁶ is unsubstituted propyl. In aspects, R⁶ is unsubstituted isopropyl. In aspects, R⁶ is unsubstituted tert-butyl. In aspects, R⁶ is substituted or unsubstituted heteroalkyl (e.g., 2 to 8 membered, 2 to 6 membered, 4 to 6 membered, 2 to 3 membered, or 4 to 5 membered). In aspects, R⁶ is substituted heteroalkyl (e.g., 2 to 8 membered, 2 to 6 membered, 4 to 6 membered, 2 to 3 membered, or 4 to 5 membered). In aspects, R⁶ is unsubstituted heteroalkyl (e.g., 2 to 8 membered, 2 to 6 membered, 4 to 6 membered, 2 to 3 membered, or 4 to 5 membered). In aspects, R⁶ is substituted or unsubstituted cycloalkyl (e.g., C₃-C₈, C₃-C₆, C₄-C₆, or C₅-C₆). In aspects, R⁶ is substituted cycloalkyl (e.g., C₃-C₈, C₃-C₆, C₄-C₆, or C₅-C₆). In aspects, R⁶ is unsubstituted cycloalkyl (e.g., C₃-C₈, C₃-C₆, C₄-C₆, or C₅-C₆). In aspects, R⁶ is substituted or unsubstituted heterocycloalkyl (e.g., 3 to 8 membered, 3 to 6 membered, 4 to 6 membered, 4 to 5 membered, or 5 to 6 membered). In aspects, R⁶ is substituted heterocycloalkyl (e.g., 3 to 8 membered, 3 to 6 membered, 4 to 6 membered, 4 to 5 membered, or 5 to 6 membered). In aspects, R⁶ is unsubstituted heterocycloalkyl (e.g., 3 to 8 membered, 3 to 6 membered, 4 to 6 membered, 4 to 5 membered, or 5 to 6 membered). In aspects, R⁶ is substituted or unsubstituted aryl (e.g., C₆-C₁₀ or phenyl). In aspects, R⁶ is substituted aryl (e.g., C₆-C₁₀ or phenyl). In aspects, R⁶ is unsubstituted aryl (e.g., C₆-C₁₀ or phenyl). In aspects, R⁶ is substituted or unsubstituted heteroaryl (e.g., 5 to 10 membered, 5 to 9 membered, or 5 to 6 membered). In aspects, R⁶ is substituted heteroaryl (e.g., 5 to 10 membered, 5 to 9 membered, or 5 to 6 membered). In aspects, R⁶ is unsubstituted heteroaryl (e.g., 5 to 10 membered, 5 to 9 membered, or 5 to 6 membered).

E is an electrophilic moiety. In aspects, E is a covalent cysteine modifier moiety. In aspects, E is:

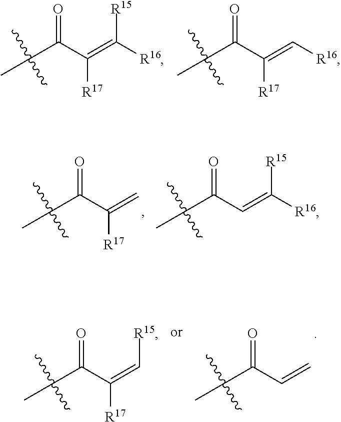

In aspects, E is:

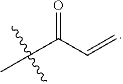 [SMILES: *C(=O)C=C]

In aspects, E is:

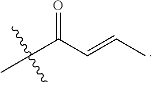 [SMILES: *C(=O)/C=C/C ; "."]

In aspects, E is:

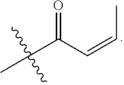 [SMILES: *C(=O)/C=C\C]

In aspects, E is:

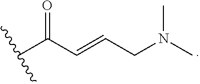 [SMILES: *C(=O)/C=C/CN(C)C ; "."]

In aspects, E is —C(O)CH═CH₂, —C(O)CH═CHCH₂N(CH₃)₂, —C(O)C(═CH₂)CH₂N(CH₃)₂, —C(O)C≡CCH₃, or —C(O)C(═CH₂)CH₃.

R^{1A}, R^{1B}, R^{6A}, and R^{6B} are each independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl.

In aspects, R^{1a}, R^{1b}, R^{6a}, and R^{6b} are each independently hydrogen, —CX^{1a}₃, —CHX^{1a}₂, —CH₂X^{1A}, —CN, —COOH, —CONH₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In aspects, R^{1A}, R^{1B}, R^{6A}, and R^{6B} are each independently hydrogen, —CX^{1A}₃, —CHX^{1A}₂, —CH₂X^{1A}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In aspects, X^{1A} is independently —F, —Cl, —Br, or —I.

In aspects, one or more of R^{1A}, R^{1B}, R^{6A}, and R^{6B} are hydrogen. In aspects, one or more of R^{1A}, R^{1B}, R^{6A}, and R^{6B} are —CX⁶₃. In aspects, one or more of R^{1A}, R^{1B}, R^{6A}, and R^{6B} are CHX⁶₂. In aspects, one or more of R^{1A}, R^{1B}, R^{6A}, and R^{6B} are —CH₂X⁶. In aspects, one or more of R^{1A}, R^{1B}, R^{6A}, and R^{6B} are —CN. In aspects, one or more of R^{1A}, R^{1B}, R^{6A}, and R^{6B} are —COOH. In aspects, one or more of R^{1A}, R^{1B}, R^{6A}, and R^{6B} are —CONH₂.

In aspects, one or more of R^{1A}, R^{1B}, R^{6A}, and R^{6B} are substituted or unsubstituted alkyl (e.g., C₁-C₈, C₁-C₆, C₁-C₄, or C₁-C₂). In aspects, one or more of R^{1A}, R^{1B}, R^{6A}, and R^{6B} are substituted alkyl (e.g., C₁-C₈, C₁-C₆, C₁-C₄, or C₁-C₂). In aspects, one or more of R^{1A}, R^{1B}, R^{6A}, and R^{6B} are unsubstituted alkyl (e.g., C₁-C₈, C₁-C₆, C₁-C₄, or C₁-C₂). In aspects, one or more of R^{1A}, R^{1B}, R^{6A}, and R^{6B} are unsubstituted methyl. In aspects, one or more of R^{1A}, R^{1B}, R^{6A} and R^{6B} are unsubstituted ethyl. In aspects, one or more of R^{1A}, R^{1B}, R^{6A}, and R^{6B} are unsubstituted propyl. In aspects, one or more of R^{1A}, R^{1B}, R^{6A}, and R^{6B} are unsubstituted isopropyl. In aspects, one or more of R^{1A}, R^{1B}, R^{6A}, and R^{6B} are unsubstituted tert-butyl. In aspects, one or more of R^{1A}, R^{1B}, R^{6A}, and R^{6B} are substituted or unsubstituted heteroalkyl (e.g., 2 to 8 membered, 2 to 6 membered, 4 to 6 membered, 2 to 3 membered, or 4 to 5 membered). In aspects, one or more of R^{1A}, R^{1B}, R^{6A}, and R^{6B} are substituted heteroalkyl (e.g., 2 to 8 membered, 2 to 6 membered, 4 to 6 membered, 2 to 3 membered, or 4 to 5 membered). In aspects, one or more of R^{1A}, R^{1B}, R^{6A}, and R^{6B} are unsubstituted heteroalkyl (e.g., 2 to 8 membered, 2 to 6 membered, 4 to 6 membered, 2 to 3 membered, or 4 to 5 membered). In aspects, one or more of R^{1A}, R^{1B}, R^{6A} and R^{6B} are substituted or unsubstituted cycloalkyl (e.g., C₃-C₈, C₃-C₆, C₄-C₆, or C₅-C₆). In aspects, one or more of R^{1A}, R^{1B}, R^{6A}, and R^{6B} are substituted cycloalkyl (e.g., C₃-C₈, C₃-C₆, C₄-C₆, or C₅-C₆). In aspects, one or more of R^{1A}, R^{1B}, R^{6A}, and R^{6B} are unsubstituted cycloalkyl (e.g., C₃-C₈, C₃-C₆, C₄-C₆, or C₅-C₆). In aspects, one or more of R^{1A}, R^{1B}, R^{6A}, and R^{6B} are substituted or unsubstituted heterocycloalkyl (e.g., 3 to 8 membered, 3 to 6 membered, 4 to 6 membered, 4 to 5 membered, or 5 to 6 membered). In aspects, one or more of R^{1A}, R^{1B}, R^{6A}, and R^{6B} are substituted heterocycloalkyl (e.g., 3 to 8 membered, 3 to 6 membered, 4 to 6 membered, 4 to 5 membered, or 5 to 6 membered). In aspects, one or more of R^{1A}, R^{1B}, R^{6A}, and R^{6B} are unsubstituted heterocycloalkyl (e.g., 3 to 8 membered, 3 to 6 membered, 4 to 6 membered, 4 to 5 membered, or 5 to 6 membered). In aspects, one or more of R^{1A}, R^{1B}, R^{6A}, and R^{6B} are substituted or unsubstituted aryl (e.g., C₆-C₁₀ or phenyl). In aspects, one or more of R^{1A}, R^{1B}, R^{6A}, and R^{6B} are substituted aryl (e.g., C₆-C₁₀ or phenyl). In aspects, one or more of R^{1A}, R^{1B}, R^{6A}, and R^{6B} are unsubstituted aryl (e.g., C₆-C₁₀ or phenyl). In aspects, one or more of R^{1A}, R^{1B}, R^{6A}, and R^{6B} are substituted or unsubstituted heteroaryl (e.g., 5 to 10 membered, 5 to 9 membered, or 5 to 6 membered). In aspects, one or more of R^{1A}, R^{1B}, R^{6A}, and R^{6B} are substituted heteroaryl (e.g., 5 to 10 membered, 5 to 9 membered, or 5 to 6 membered). In aspects, o one or more of R^{1A}, R^{1B}, R^{6A}, and R^{6B} are unsubstituted heteroaryl (e.g., 5 to 10 membered, 5 to 9 membered, or 5 to 6 membered).

In aspects, one or more of R^{1A}, R^{1B}, R^{6A}, and R^{6B} are hydrogen. In aspects, one or more of R^{1A}, R^{1B}, R^{6A}, and R^{6B} are methyl. In aspects, one or more of R^{1A}, R^{1B}, R^{6A}, and R^{6B} are ethyl. In aspects, one or more of R^{1A}, R^{1B}, R^{6A}, and R^{6B} are propyl. In aspects, one or more of R^{1A}R^{1B}, R^{6A}, and R^{6B} are —CN. In as one or more of R^{1A}, R^{1B}, R^{6A}, and R^{6B} are —CONH₂. In aspects, one or more of R^{1A}, R^{1B}, R^{6A}, and R^{6B} are —F, —Cl, —Br, or —I.

X, X¹, X^{1A}, X⁶, and X¹⁵ are each independently —F, —Cl, —Br, or —I. In aspects, X is —F. In aspects, X is —Cl. In aspects, X is —Br. In aspects, X is —I. In aspects, X¹ is —F. In aspects, X¹ is —Cl. In aspects, X¹ is —Br. In aspects, X¹ is —I. In aspects, X^{1A} is —F. In aspects, X^{1A} is —Cl. In aspects, X^{1A} is —Br. In aspects, X^{1A} is —I. In aspects, X⁶ is —F. In aspects, X⁶ is —Cl. In aspects, X⁶ is —Br. In aspects, X⁶ is —I. In aspects, X¹⁵ is —F. In aspects, X¹⁵ is —Cl. In aspects, X¹⁵ is —Br. In aspects, X¹⁵ is —I.

n1 is each independently an integer from 0 to 4. In aspects, n1 is 0. In aspects, n1 is 1. In aspects, n1 is 2. In aspects, n1 is 3. In aspects, n1 is 4.

m1 is independently 1 or 2. In aspects, m1 is 1. In aspects, m1 is 2.

v1 is independently 1 or 2. In aspects, v1 is 1. In aspects, v1 is 2.

R¹⁵, R¹⁶, and R¹⁷ are each independently hydrogen, halogen, —CX¹⁵³, —CHX¹⁵₂ —CH₂X¹⁵, —CN, —SOₙ₁₅R^{15A}, —SOᵥ₁₅NR^{15A}R^{15B}, —NHNR^{15A}R^{15B}, —ONR^{15A}R^{15B}, —NHC=(O)NHNR^{15A}R^{15B}, —NHC(O)NR^{15A}R^{15B}, —N(O)ₘ₁₅, —NR^{15A}R^{15B}, —C(O)R^{15A}, —C(O)—OR^{15A}, —C(O)NR^{15A}R^{15B}, OR^{15A}, —NR^{15A}SO₂R^{15B}, —NR^{15A}C(O)R^{15B}, —R^{15A}C(O)OR^{15B}, —NR^{15A}OR^{15B}, —OCX^{15B}, —OCHX¹⁵₂, —OCH₂X¹⁵, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In aspects, one or more of R¹⁵, R¹⁶, and R¹⁷ are hydrogen. In aspects, one or more of R¹⁵, R¹⁶, and R¹⁷ are halogen. In aspects, one or more of R¹⁵, R¹⁶, and R¹⁷ are —CX¹⁵₃. In aspects, one or more of R¹⁵, R¹⁶, and R¹⁷ are —CHX¹₂. In aspects, one or more of R¹⁵, R¹⁶, and R¹⁷ are —CH₂X¹⁵. In aspects, one or more of R¹⁵, R¹⁶, and R¹⁷ are —CN. In aspects, one or more of R¹⁵, R¹⁶, and R¹⁷ are —SOₙ₁₅R^{15A}. In aspects, one or more of R¹⁵, R¹⁶, and R¹⁷ are —SOᵥ₁₅NR^{15A}R^{15B}. In aspects, one or more of R¹⁵, R¹⁶, and R¹⁷ are —NHNR^{15A}R^{15B}. In aspects, one or more of R¹⁵, R¹⁶, and R¹⁷ are —ONR^{15A}R^{15B}. In aspects, one or more of R¹⁵, R¹⁶, and R¹⁷ are —NHC=(O)NHNR^{15A}R^{15B}. In aspects, one or more of R¹⁵, R¹⁶, and R¹⁷ are —NHC(O)NR^{15A}R^{15B}. In aspects, one or more of R¹⁵, R¹⁶, and R¹⁷ are —N(O)ₘ₁₅. In aspects, one or more of R¹⁵, R¹⁶, and R¹⁷ are —NR^{15A}R^{15B}. In aspects, one or more of R¹⁵, R¹⁶, and R¹⁷ are —C(O)R^{15A}. In aspects, one or more of R¹⁵, R¹⁶, and R¹⁷ are —C(O)—OR^{15A}. In aspects, one or more of R¹⁵, R¹⁶, and R¹⁷ are —C(O)NR^{15A}R^{15B}. In aspects, one or more of R¹⁵, R¹⁶, and R¹⁷ are —OR^{15A}. In aspects, one or more of R¹⁵, R¹⁶, and R¹⁷ are —NR^{15A}SO₂R^{15B}. In aspects, one or more of R¹⁵, R¹⁶, and R¹⁷ are —NR^{15A}C(O)R^{15B}. In aspects, one or more of R¹⁵, R¹⁶, and R¹⁷ are —NR^{15A}C(O)OR^{15B} In aspects, one or more of R¹⁵, R¹⁶, and R¹⁷ are —NR^{15A}OR^{15B}. In aspects, one or more of R¹⁵, R¹⁶, and R¹⁷ are —OCX¹⁵₃. In aspects, one or more of R¹⁵, R¹⁶, and R¹⁷ are —OCHX¹⁵₂. In aspects, one or more of R¹⁵, R¹⁶, and R¹⁷ are —OCH₂X¹⁵. In aspects, one or more of R¹⁵, R¹⁶, and R¹⁷ are —OH. In aspects, one or more of R¹⁵, R¹⁶, and R¹⁷ are —NH₂. In aspects, one or more of R¹⁵, R¹⁶, and R¹⁷ are —COOH. In aspects, one or more of R¹⁵, R¹⁶, and R¹⁷ are —CONH₂. In aspects, one or more of R¹⁵, R¹⁶, and R¹⁷ are —NO₂. In aspects, one or more of R¹⁵, R¹⁶, and R¹⁷ are —SH. In aspects, one or more of R¹⁵, R¹⁶, and R¹⁷ are —CF₃. In aspects, one or more of R¹⁵, R¹⁶, and R¹⁷ are —CHF₂. In aspects, one or more of R¹⁵, R¹⁶, and R¹⁷ are —CH₂F. In aspects, one or more of R¹⁵, R¹⁶, and R¹⁷ are —OCF₃. In aspects, one or more of R¹⁵, R¹⁶, and R¹⁷ are —OCH₂F. In aspects, one or more of R¹⁵, R¹⁶, and R¹⁷ are —OCHF₂. In aspects, one or more of R¹⁵, R¹⁶, and R¹⁷ are —OCH₃. In aspects, one or more of R¹⁵, R¹⁶, and R¹⁷ are —OCH₂CH₃. In aspects, one or more of R¹⁵, R¹⁶, and R¹⁷ are —OCH₂CH₂CH₃. In aspects, one or more of R¹⁵, R¹⁶, and R¹⁷ are —OCH(CH₃)₂. In aspects, one or more of R¹⁵, R¹⁶, and R¹⁷ are —OC(CH₃)₃. In aspects, one or more of R¹⁵, R¹⁶, and R¹⁷ are —SCH₃. In aspects, one or more of R¹⁵, R¹⁶, and R¹⁷ are —SCH₂CH₃. In aspects, one or more of R¹⁵, R¹⁶, and R¹⁷ are —SCH₂CH₂CH₃. In aspects, one or more of R¹⁵, R¹⁶, and R¹⁷ are SCH(CH₃)₂. In aspects, one or more of R¹⁵, R¹⁶, and R¹⁷ are —SC(CH₃)₃. In aspects, one or more of R¹⁵, R¹⁶, and R¹⁷ are —CH₃. In aspects, one or more of R¹⁵, R¹⁶, and R¹⁷ are —CH₂CH₃. In aspects, one or more of R¹⁵, R¹⁶, and R¹⁷ are —CH₂CH₂CH₃. In aspects, one or more of R⁵, R¹⁶, and R¹⁷ are —CH(CH₃)₂. In aspects, one or more of R¹⁵, R¹⁶, and R¹⁷ are —C(CH₃)₃. In aspects, one or more of R¹⁵, R¹⁶, and R¹⁷ are —F. In aspects, one or more of R¹⁵, R¹⁶, and R¹⁷ are —Cl. In aspects, one or more of R¹⁵, R¹⁶, and R¹⁷ are —Br. In aspects, one or more of R¹⁵, R¹⁶, and R¹⁷ are —I.

In aspects, one or more of R¹⁵, R¹⁶, and R¹⁷ are substituted or unsubstituted alkyl (e.g., C₁-C₈, C₁-C₆, C₁-C₄, or C₁-C₂). In aspects, one or more of R¹⁵, R¹⁶, and R¹⁷ are substituted alkyl (e.g., C₁-C₈, C₁-C₆, C₁-C₄, or C₁-C₂). In aspects, one or more of R¹⁵, R¹⁶, and R¹⁷ are unsubstituted alkyl (e.g., C₁-C₈, C₁-C₆, C₁-C₄, or C₁-C₂). In aspects, one or more of R¹⁵, R¹⁶, and R¹⁷ are unsubstituted methyl. In aspects, one or more of R¹⁵, R¹⁶, and R¹⁷ are unsubstituted ethyl. In aspects, one or more of R¹⁵, R¹⁶, and R¹⁷ are unsubstituted propyl. In aspects, one or more of R¹⁵, R¹⁶, and R¹⁷ are unsubstituted isopropyl. In aspects, one or more of R¹⁵, R¹⁶, and R¹⁷ are unsubstituted tert-butyl. In aspects, one or more of R¹⁵, R¹⁶, and R¹⁷ are substituted or unsubstituted heteroalkyl (e.g., 2 to 8 membered, 2 to 6 membered, 4 to 6 membered, 2 to 3 membered, or 4 to 5 membered). In aspects, one or more of R¹⁵, R¹⁶, and R¹⁷ are substituted heteroalkyl (e.g., 2 to 8 membered, 2 to 6 membered, 4 to 6 membered, 2 to 3 membered, or 4 to 5 membered). In aspects, one or more of R¹⁵, R¹⁶, and R¹⁷ are unsubstituted heteroalkyl (e.g., 2 to 8 membered, 2 to 6 membered, 4 to 6 membered, 2 to 3 membered, or 4 to 5 membered). In aspects, one or more of R¹⁵, R¹⁶, and R¹⁷ are substituted or unsubstituted cycloalkyl (e.g., C₃-C₅, C₃-C₆, C₄-C₆, or C₅-C₆). In aspects, one or more of R¹⁵, R¹⁶, and R¹⁷ are substituted cycloalkyl (e.g., C₃-C₈, C₃-C₆, C₄-C₆, or C₅-C₆). In aspects, R¹⁵ is unsubstituted cycloalkyl (e.g., C₃-C₈, C₃-C₆, C₄-C₆, or C₅-C₆). In aspects, one or more of R¹⁵, R¹⁶, and R¹⁷ are substituted or unsubstituted heterocycloalkyl (e.g., 3 to 8 membered, 3 to 6 membered, 4 to 6 membered, 4 to 5 membered, or 5 to 6 membered). In aspects, one or more of R¹⁵, R¹⁶, and R¹⁷ are substituted heterocycloalkyl (e.g., 3 to 8 membered, 3 to 6 membered, 4 to 6 membered, 4 to 5 membered, or 5 to 6 membered). In aspects, one or more of R¹⁵, R¹⁶, and R¹⁷ are unsubstituted heterocycloalkyl (e.g., 3 to 8 membered, 3 to 6 membered, 4 to 6 membered, 4 to 5 membered, or 5 to 6 membered). In aspects, one or more of R¹⁵, R¹⁶, and R¹⁷ are substituted or unsubstituted aryl (e.g., C₆-C₁₀ or phenyl). In aspects, one or more of R¹⁵, R¹⁶, and R¹⁷ are substituted aryl (e.g., C₆-C₁₀ or phenyl). In aspects, one or more of R¹⁵, R¹⁶, and R¹⁷ are unsubstituted aryl (e.g., C₆-C₁₀ or phenyl). In aspects, one or more of R¹⁵, R¹⁶, and R¹⁷ are substituted or unsubstituted heteroaryl (e.g., 5 to 10 membered, 5 to 9 membered, or 5 to 6 membered). In aspects, one or more of R¹⁵, R¹⁶, and R¹⁷ are substituted heteroaryl (e.g., 5 to 10 membered, 5 to 9 membered, or 5 to 6 membered). In aspects, one or more of R¹⁵, R¹⁶, and R¹⁷ are unsubstituted heteroaryl (e.g., 5 to 10 membered, 5 to 9 membered, or 5 to 6 membered).

In aspects, R¹⁵, R¹⁶, and R¹⁷ are each independently hydrogen, halogen, —CX¹⁵₃, —CHX¹⁵₂, —CH₂X¹⁵, —OCX¹⁵₃, —OCH₂X¹⁵, OCHX¹⁵₂, —CN, —OH, —NH₂, —COOH, —CONH₂, —NO₂, —SH, —SO₃H, —SO₄H, —SO₂NH₂, —NHNH₂, —ONH₂, —NHC=(O)NHNH₂, —NHC═(O)NH₂, —NHSO₂H, —NHC═(O)H, —NHC(O)—OH, —NHOH, R^{15A}-substituted or unsubstituted alkyl (e.g., C₁-C₈, C₁-C₆, C₁-C₄, or C₁-C₂), In aspects, R¹⁵, R¹, and R¹⁷ are each independently hydrogen, halogen, —CX¹⁵₃, —CHX¹⁵₂, —CH₂X¹⁵, —OCX¹⁵₃, —OCH₂X¹⁵, —OCHX¹⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In aspects, one or more of R¹⁵, R¹⁶, and R¹⁷ are hydrogen. In aspects, one or more of R¹⁵, R¹⁶, and R¹⁷ are unsubstituted methyl. In aspects, one or more of R¹⁵, R¹⁶, and R¹⁷ are unsubstituted ethyl.

In aspects, R¹⁵, R¹⁶, and R¹⁷ are hydrogen. In aspects, R¹⁵ is hydrogen. In aspects, R¹⁶ is hydrogen, —CH₃, or —CH₂NR^{15A}R^{15B}. In aspects, R¹⁷ is hydrogen. In aspects, R^{15A} and R^{15B} are independently hydrogen or unsubstituted alkyl. In aspects, R^{15A} and R^{15B} are independently unsubstituted methyl. In aspects. R^{15A} is hydrogen. In aspects, R^{15A} is unsubstituted alkyl. In aspects, R^{15B} is hydrogen. In aspects, R^{15B} is unsubstituted alkyl. In aspects, R^{15A} is unsubstituted methyl. In aspects, R^{15B} is unsubstituted methyl. In aspects, R¹⁵ is hydrogen; R¹⁶ is hydrogen, —CH₃, or —CH₂NR^{15A}R^{15B}; R¹⁷ is hydrogen; and R^{15A} and R^{15B} are independently hydrogen or unsubstituted alkyl.

In aspects, R¹⁵ is hydrogen. In aspects, R¹⁶ is hydrogen. In aspects, R¹⁷ is hydrogen, —CH₃, or —CH₂NR^{15A}R^{15B}. In aspects, R^{15A} and R^{15B} are independently hydrogen or unsubstituted alkyl. In aspects, R^{15A} and R^{15B} are independently unsubstituted methyl. In aspects, R¹⁵ is hydrogen; R¹⁶ is hydrogen; R¹⁷ is hydrogen, —CH₃, or —CH₂NR^{15A}R^{15B}, and R^{15A} and R^{15B} are independently hydrogen or unsubstituted alkyl. In aspects, R^{15A} is hydrogen. In aspects, R^{15A} is unsubstituted alkyl. In aspects, R^{15B} is hydrogen. In aspects, R^{15B} is unsubstituted alkyl. In aspects, R^{15A} is unsubstituted methyl. In aspects, R^{15B} is unsubstituted methyl.

In aspects, R¹⁵ is hydrogen, —CH₃, or —CH₂NR^{15A}R^{15B}. In aspects, R¹⁶ is hydrogen. In aspects, R¹⁷ is hydrogen. In aspects, R^{15A} and R^{15B} are independently hydrogen or unsubstituted alkyl. In aspects, R^{15A} and R^{15B} are independently unsubstituted methyl. In aspects, R^{15A} is hydrogen. In aspects, R^{15A} is unsubstituted alkyl. In aspects, R^{15B} is hydrogen. In aspects, R^{15B} is unsubstituted alkyl. In aspects, R^{15A} is unsubstituted methyl. In aspects, R^{15B} is unsubstituted methyl. In aspects, R¹⁵ is hydrogen, —CH₃, or —CH₂NR^{15A}R^{15B}; R¹⁶ is hydrogen; R¹⁷ is hydrogen; and R^{15A} and R^{15B} are independently hydrogen or unsubstituted alkyl.

R^{15A} and R^{15B} are each independently hydrogen, —CX₃, —CN, —COOH, —CONH₂, —CHX₂, —CH₂X, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{15A} and R^{15B} substituents bonded to the same nitrogen atom may optionally be joined to form a substituted or unsubstituted heterocycloalkyl (e.g., 2 to 8 membered, 2 to 6 membered, 4 to 6 membered, 2 to 3 membered, or 4 to 5 membered) or substituted or unsubstituted heteroaryl (e.g., 5 to 10 membered, 5 to 9 membered, or 5 to 6 membered).

In aspects, one or both of R^{15A} and R^{15B} are hydrogen. In aspects, one or both of R^{15A} and R^{15B} are —CX^{15A}₃. In aspects, one or both of R^{15A} and R^{15B} are —CHX^{15A}₂. In aspects, one or both of R^{15A} and R^{15B} are —CH₂X^{15A}. In aspects, one or both of R^{15A} and R^{15B} are —CN. In aspects, one or both of R^{15A} and R^{15B} are —COOH. In aspects, one or both of R^{15A} and R^{15B} are —CONH₂.

In aspects, one or both of R^{15A} and R^{15B} are substituted or unsubstituted alkyl (e.g., C₁-C₈, C₁-C₆, C₁-C₄, or C₁-C₂). In aspects, one or both of R^{15A} and R^{15B} are substituted alkyl (e.g., C₁-C₈, C₁-C₆, C₁-C₄, or C₁-C₂). In aspects, one or both of R^{15A} and R^{15B} are unsubstituted alkyl (e.g., C₁-C₈, C₁-C₆, C₁-C₄, or C₁-C₂). In aspects, one or both of R^{15A} and R^{15B} are unsubstituted methyl. In aspects, one or both of R^{15A} and R^{15B} are unsubstituted ethyl. In aspects, one or both of R^{15A} and R^{15B} are unsubstituted propyl. In aspects, one or both of R^{15A} and R^{15B} are unsubstituted isopropyl. In aspects, one or both of R^{15A} and R^{15B} are unsubstituted tert-butyl. In aspects, one or both of R^{15A} and R^{15B} are substituted or unsubstituted heteroalkyl (e.g., 2 to 8 membered, 2 to 6 membered, 4 to 6 membered, 2 to 3 membered, or 4 to 5 membered). In aspects, one or both of R^{15A} and R^{15B} are substituted heteroalkyl (e.g., 2 to 8 membered, 2 to 6 membered, 4 to 6 membered, 2 to 3 membered, or 4 to 5 membered). In aspects, one or both of R^{15A} and R^{15B} are unsubstituted heteroalkyl (e.g., 2 to 8 membered, 2 to 6 membered, 4 to 6 membered, 2 to 3 membered, or 4 to 5 membered). In aspects, one or both of R^{15A} and R^{15B} are substituted or unsubstituted cycloalkyl (e.g., C₃-C₈, C₃-C₆, C₄-C₆, or C₅-C₆). In aspects, one or both of R^{15A} and R^{15B} are substituted cycloalkyl (e.g., C₃-C₈, C₃-C₆, C₄-C₆, or C₅-C₆). In aspects, one or both of R^{15A} and R^{15B} are unsubstituted cycloalkyl (e.g., C₃-C₈, C₃-C₆, C₄-C₆, or C₅-C₆). In aspects, one or both of R^{15A} and R^{15B} are substituted or unsubstituted heterocycloalkyl (e.g., 3 to 8 membered, 3 to 6 membered, 4 to 6 membered, 4 to 5 membered, or 5 to 6 membered). In aspects, one or both of R^{15A} and R^{15B} are substituted heterocycloalkyl (e.g., 3 to 8 membered, 3 to 6 membered, 4 to 6 membered, 4 to 5 membered, or 5 to 6 membered). In aspects, one or both of R^{15A} and R^{15B} are unsubstituted heterocycloalkyl (e.g., 3 to 8 membered, 3 to 6 membered, 4 to 6 membered, 4 to 5 membered, or 5 to 6 membered). In aspects, one or both of R^{15A} and R^{15B} are substituted or unsubstituted aryl (e.g., C₆-C₁₀ or phenyl). In aspects, one or both of R^{15A} and R^{15B} are substituted aryl (e.g., C₆-C₁₀ or phenyl). In aspects, one or both of R^{15A} and R^{15B} are unsubstituted aryl (e.g., C₆-C₁₀ or phenyl). In aspects, one or both of R^{15A} and R^{15B} are substituted or unsubstituted heteroaryl (e.g., 5 to 10 membered, 5 to 9 membered, or 5 to 6 membered). In aspects, one or both of R^{15A} and R^{15B} are substituted heteroaryl (e.g., 5 to 10 membered, 5 to 9 membered, or 5 to 6 membered). In aspects, one or both of R^{15A} and R^{15B} are unsubstituted heteroaryl (e.g., 5 to 10 membered, 5 to 9 membered, or 5 to 6 membered).

In aspects, R^{15A} and R^{15B} are each independently hydrogen, —CX^{15A}₃, —CHX^{15A}₂, —CH₂X^{15A}, —CN, —COOH, —CONH₂, In aspects, R^{15A} and R^{15B} are each independently hydrogen, —CX^{15A}₃, —CHX^{15A}₂, —CH₂X^{l5A}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In aspects, X^{15A} is independently —F, —Cl, —Br, or —I. In aspects, one or both of R^{15A} and R^{15B} are hydrogen. In aspects, one or both of R^{15A} and R^{15B} are unsubstituted methyl. In aspects, one or both of R^{15A} and R^{15B} are unsubstituted ethyl.

n15 is independently an integer from 0 to 4. In aspects, n15 is 0. In aspects, n15 is 1. In aspects, n15 is 2. In aspects, n15 is 3. In aspects, n15 is 4.

v15 is 1 or 2. In aspects, v15 is 1. In aspects, v15 is 2.

m15 is 1 or 2. In aspects m15 is 1. In aspects m15 is 2.

In embodiments, the compound of Formula (I) is a compound of Formula (A) or a pharmaceutically acceptable salt thereof:

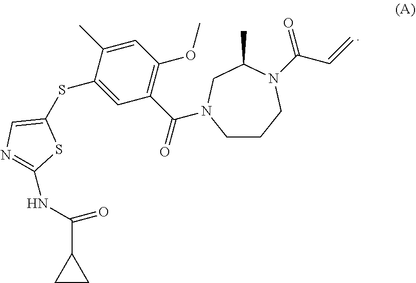 [SMILES: C=CC(=O)N1CCCN(C(=O)c2cc(Sc3cnc(NC(=O)C4CC4)s3)c(C)cc2OC)C[C@H]1C ; "."]

In embodiments, the compound of Formula (I) is a compound of any one of Formula (1) to Formula (34) or a pharmaceutically acceptable salt of any one of the foregoing:

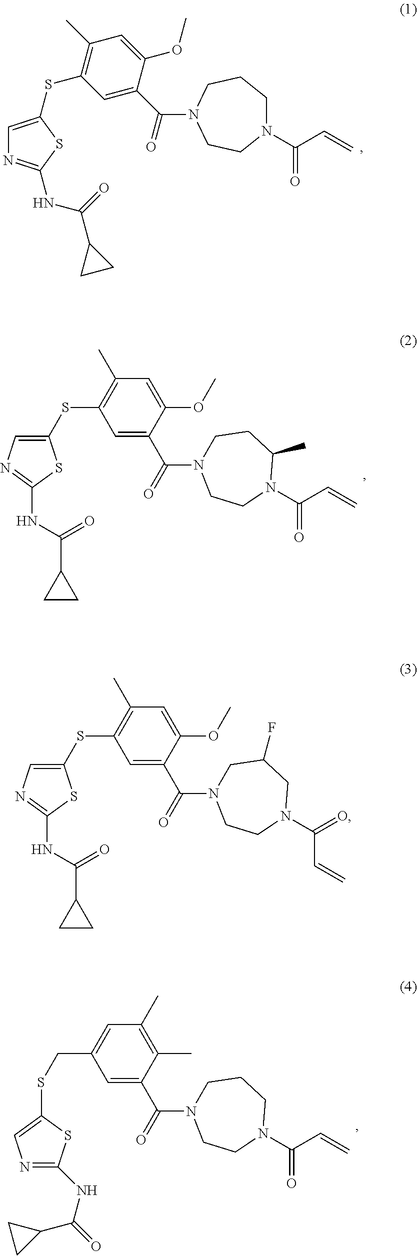 [SMILES: C=CC(=O)N1CCCN(C(=O)c2cc(Sc3cnc(NC(=O)C4CC4)s3)c(C)cc2OC)CC1 ; C=CC(=O)N1CCN(C(=O)c2cc(Sc3cnc(NC(=O)C4CC4)s3)c(C)cc2OC)CC[C@H]1C ; C=CC(=O)N1CCN(C(=O)c2cc(Sc3cnc(NC(=O)C4CC4)s3)c(C)cc2OC)CC(F)C1 ; C=CC(=O)N1CCCN(C(=O)c2cc(CSc3cnc(NC(=O)C4CC4)s3)cc(C)c2C)CC1]

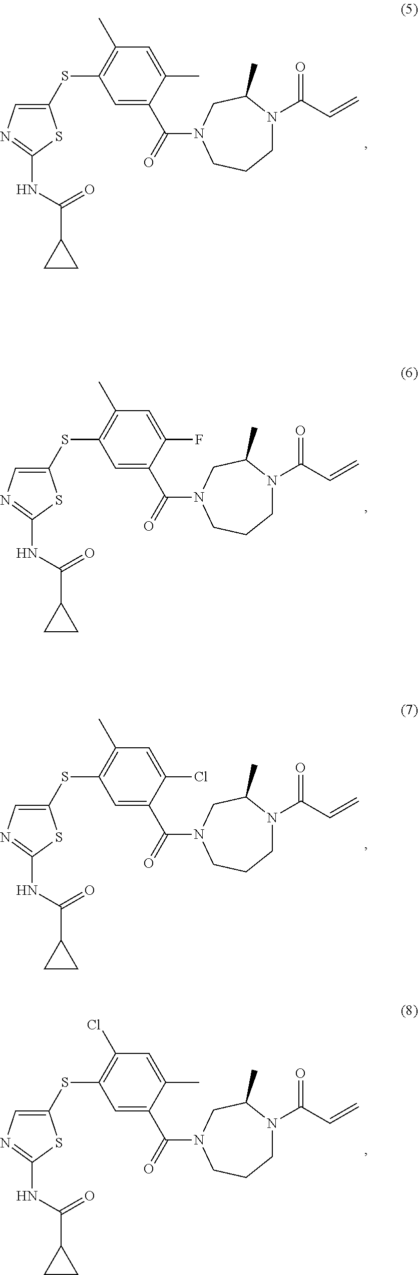 [SMILES: C=CC(=O)N1CCCN(C(=O)c2cc(Sc3cnc(NC(=O)C4CC4)s3)c(C)cc2C)C[C@H]1C ; C=CC(=O)N1CCCN(C(=O)c2cc(Sc3cnc(NC(=O)C4CC4)s3)c(C)cc2F)C[C@H]1C ; C=CC(=O)N1CCCN(C(=O)c2cc(Sc3cnc(NC(=O)C4CC4)s3)c(C)cc2Cl)C[C@H]1C ; C=CC(=O)N1CCCN(C(=O)c2cc(Sc3cnc(NC(=O)C4CC4)s3)c(Cl)cc2C)C[C@H]1C]

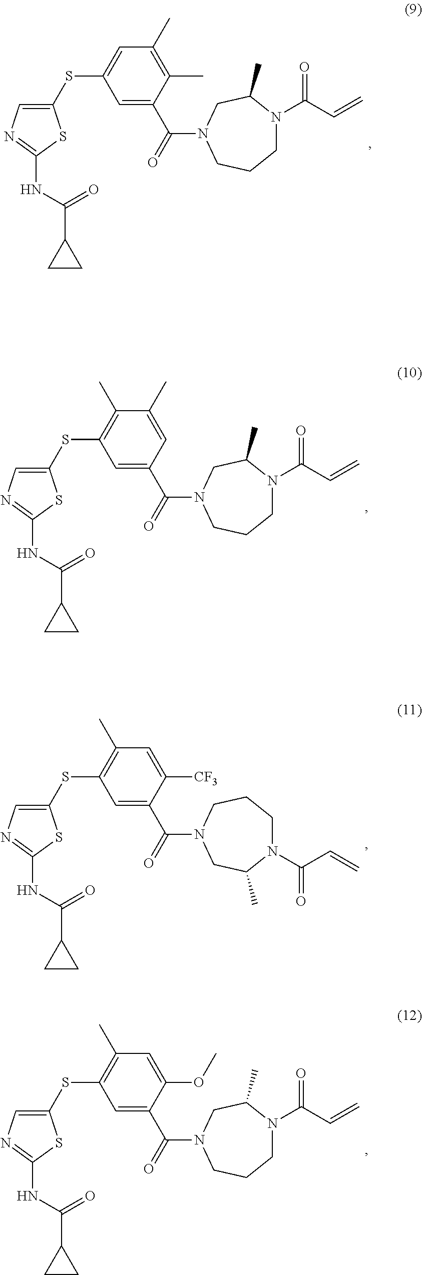 [SMILES: C=CC(=O)N1CCCN(C(=O)c2cc(Sc3cnc(NC(=O)C4CC4)s3)cc(C)c2C)C[C@H]1C ; C=CC(=O)N1CCCN(C(=O)c2cc(C)c(C)c(Sc3cnc(NC(=O)C4CC4)s3)c2)C[C@H]1C ; C=CC(=O)N1CCCN(C(=O)c2cc(Sc3cnc(NC(=O)C4CC4)s3)c(C)cc2C(F)(F)F)C[C@H]1C ; C=CC(=O)N1CCCN(C(=O)c2cc(Sc3cnc(NC(=O)C4CC4)s3)c(C)cc2OC)C[C@@H]1C]

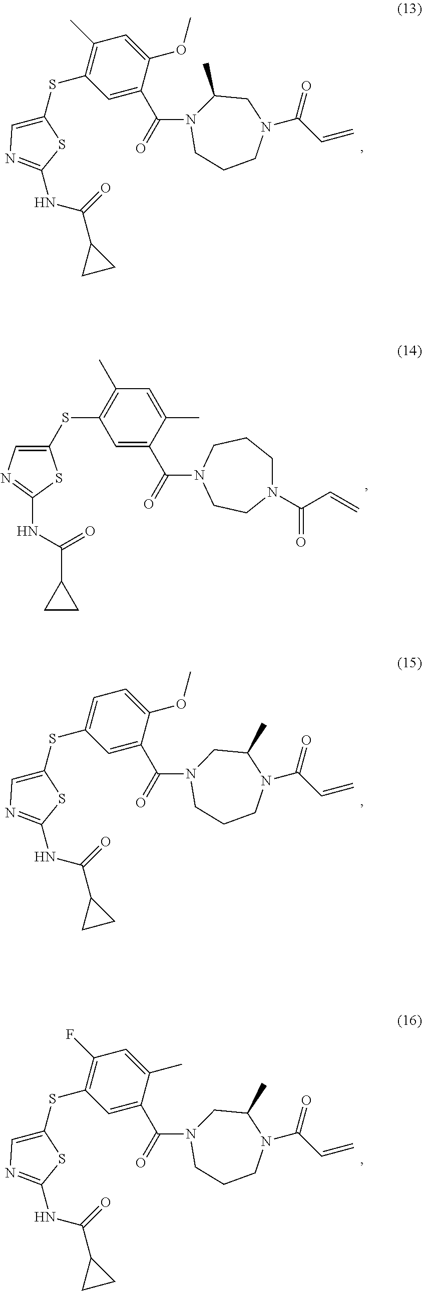 [SMILES: C=CC(=O)N1CCCN(C(=O)c2cc(Sc3cnc(NC(=O)C4CC4)s3)c(C)cc2OC)[C@@H](C)C1 ; C=CC(=O)N1CCCN(C(=O)c2cc(Sc3cnc(NC(=O)C4CC4)s3)c(C)cc2C)CC1 ; C=CC(=O)N1CCCN(C(=O)c2cc(Sc3cnc(NC(=O)C4CC4)s3)ccc2OC)C[C@H]1C ; C=CC(=O)N1CCCN(C(=O)c2cc(Sc3cnc(NC(=O)C4CC4)s3)c(F)cc2C)C[C@H]1C]

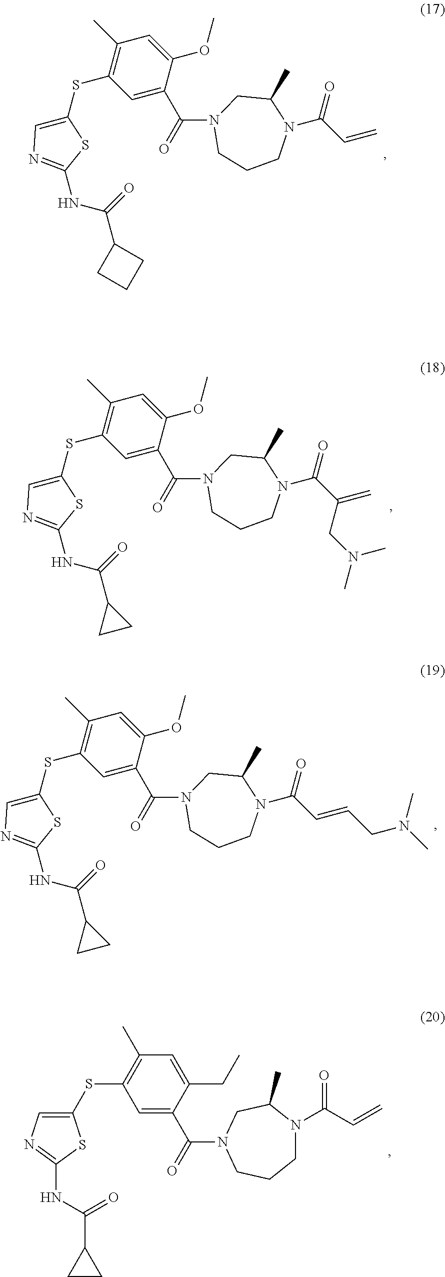 [SMILES: C=CC(=O)N1CCCN(C(=O)c2cc(Sc3cnc(NC(=O)C4CCC4)s3)c(C)cc2OC)C[C@H]1C ; C=C(CN(C)C)C(=O)N1CCCN(C(=O)c2cc(Sc3cnc(NC(=O)C4CC4)s3)c(C)cc2OC)C[C@H]1C ; COc1cc(C)c(Sc2cnc(NC(=O)C3CC3)s2)cc1C(=O)N1CCCN(C(=O)/C=C/CN(C)C)[C@H](C)C1 ; C=CC(=O)N1CCCN(C(=O)c2cc(Sc3cnc(NC(=O)C4CC4)s3)c(C)cc2CC)C[C@H]1C]

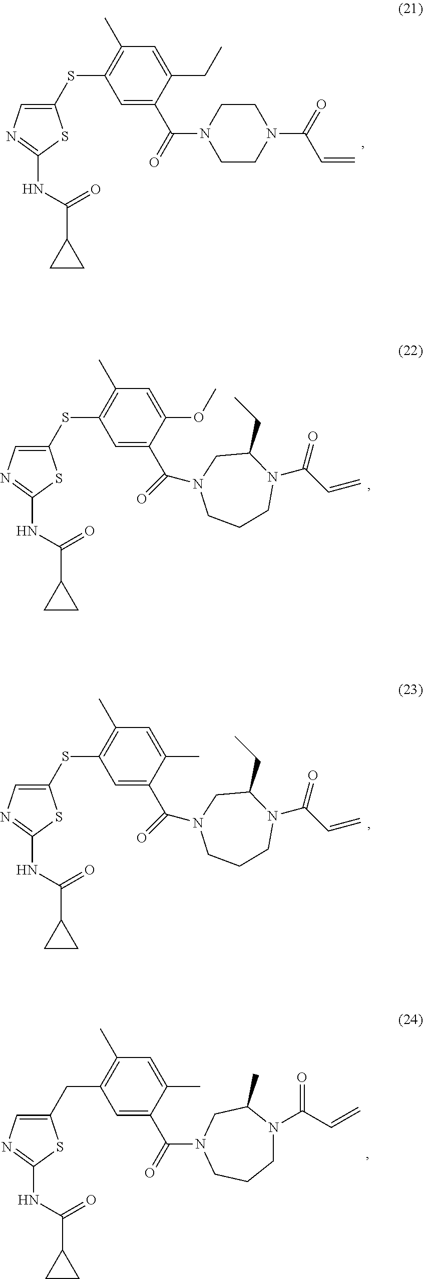 [SMILES: C=CC(=O)N1CCN(C(=O)c2cc(Sc3cnc(NC(=O)C4CC4)s3)c(C)cc2CC)CC1 ; C=CC(=O)N1CCCN(C(=O)c2cc(Sc3cnc(NC(=O)C4CC4)s3)c(C)cc2OC)C[C@H]1CC ; C=CC(=O)N1CCCN(C(=O)c2cc(Sc3cnc(NC(=O)C4CC4)s3)c(C)cc2C)C[C@H]1CC ; C=CC(=O)N1CCCN(C(=O)c2cc(Cc3cnc(NC(=O)C4CC4)s3)c(C)cc2C)C[C@H]1C]

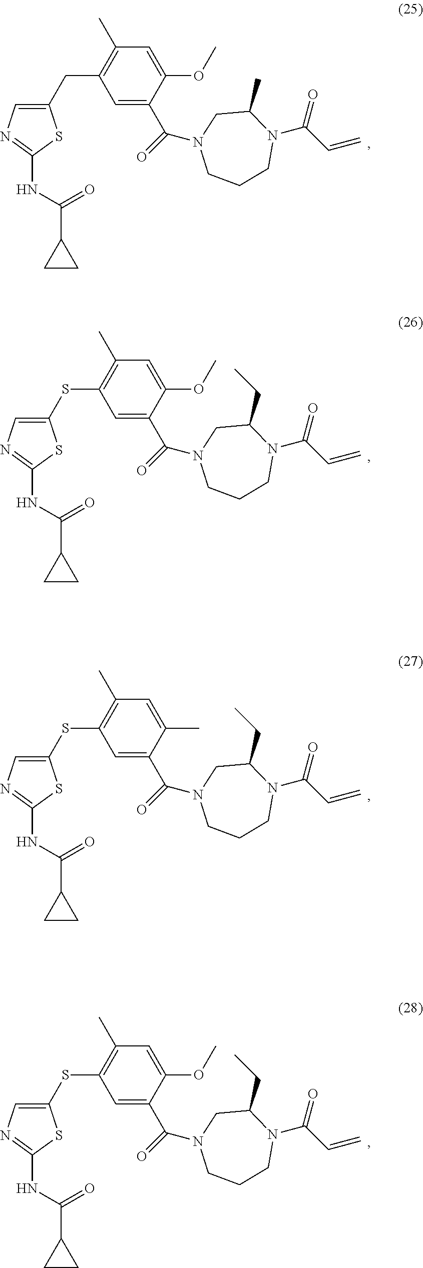 [SMILES: C=CC(=O)N1CCCN(C(=O)c2cc(Cc3cnc(NC(=O)C4CC4)s3)c(C)cc2OC)C[C@H]1C ; C=CC(=O)N1CCCN(C(=O)c2cc(Sc3cnc(NC(=O)C4CC4)s3)c(C)cc2OC)C[C@H]1CC ; C=CC(=O)N1CCCN(C(=O)c2cc(Sc3cnc(NC(=O)C4CC4)s3)c(C)cc2C)C[C@H]1CC ; C=CC(=O)N1CCCN(C(=O)c2cc(Sc3cnc(NC(=O)C4CC4)s3)c(C)cc2OC)C[C@H]1CC]

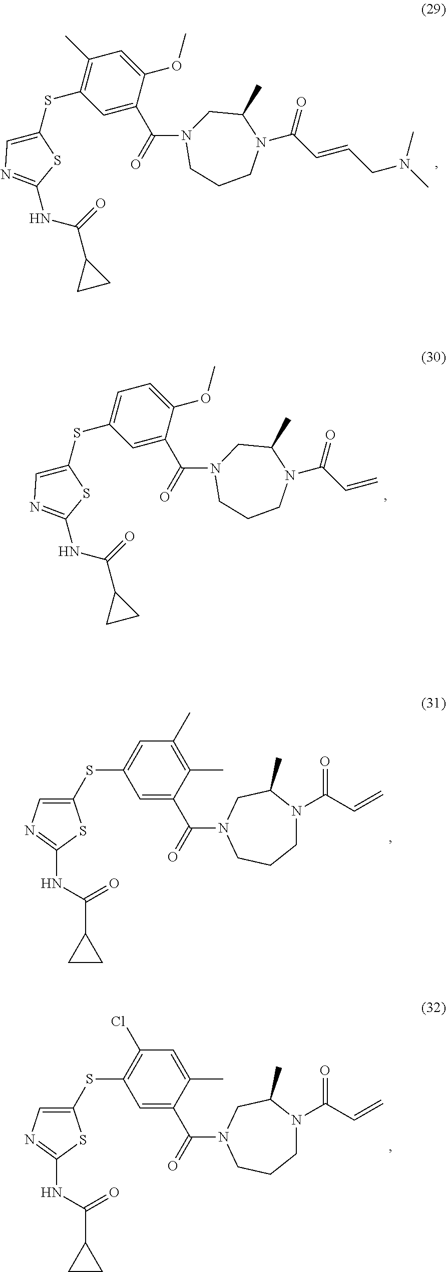 [SMILES: COc1cc(C)c(Sc2cnc(NC(=O)C3CC3)s2)cc1C(=O)N1CCCN(C(=O)/C=C/CN(C)C)[C@H](C)C1 ; C=CC(=O)N1CCCN(C(=O)c2cc(Sc3cnc(NC(=O)C4CC4)s3)ccc2OC)C[C@H]1C ; C=CC(=O)N1CCCN(C(=O)c2cc(Sc3cnc(NC(=O)C4CC4)s3)cc(C)c2C)C[C@H]1C ; C=CC(=O)N1CCCN(C(=O)c2cc(Sc3cnc(NC(=O)C4CC4)s3)c(Cl)cc2C)C[C@H]1C]

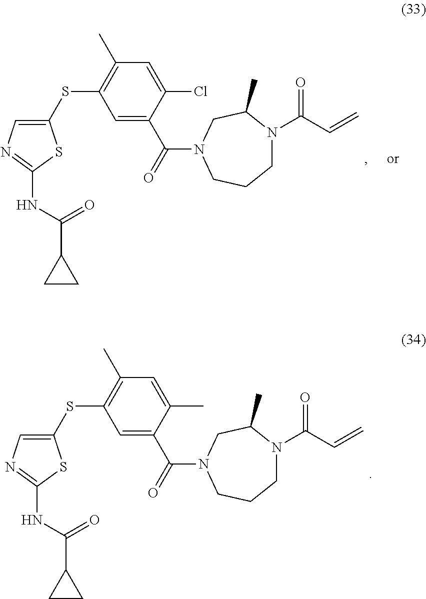 [SMILES: C=CC(=O)N1CCCN(C(=O)c2cc(Sc3cnc(NC(=O)C4CC4)s3)c(C)cc2Cl)C[C@H]1C ; C=CC(=O)N1CCCN(C(=O)c2cc(Sc3cnc(NC(=O)C4CC4)s3)c(C)cc2C)C[C@H]1C]

Methods for making the ITK inhibitors described herein are set forth in U.S. Pat. No. 11,008,314, the disclosure of which is incorporated by reference herein in its entirety.

### Methods of Treatment

The terms “treating” and “treatment” refer to any indicia of success in the therapy or amelioration of a disease, pathology or condition, including any objective or subjective parameter such as abatement; remission; diminishing of symptoms or making the pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination. The term “treating” and conjugations thereof, may include prevention of a pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an ITK inhibitor. The administering step may consist of a single administration or may include a series of administrations. The length of the treatment period depends on a variety of factors, such as the severity of the condition, the age of the patient, the concentration of the ITK inhibitor, the activity of the compositions used in the treatment, or a combination thereof. It will also be appreciated that the effective dosage of an ITK inhibitor used for the treatment or prophylaxis may increase or decrease over the course of a particular treatment or prophylaxis regime. Changes in dosage may result and become apparent by standard diagnostic assays known in the art. In instances, chronic administration may be required. For example, the compositions are administered to the subject in an amount and for a duration sufficient to treat the patient. In embodiments, the treating or treatment is not prophylactic treatment.

“Patient” or “subject” refers to a mammal suffering from or prone to a disease (e.g., cancer) that can be treated by administration of a compound or pharmaceutical composition or by a method, as provided herein. Non-limiting examples of a patient include humans, bovines, rats, mice, dogs, cats, monkeys, goat, sheep, and the like. In embodiments, a patient is human. In embodiments, a patient is a dog or a cat In embodiments, the patient is a human adult. In embodiments, the patient is a human child.

The term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal) compatible with the preparation. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Biological sample” or “sample” refer to materials obtained from or derived from a subject or patient. A biological sample includes sections of tissues such as biopsy. Such samples include bodily fluids such as blood and blood fractions or products (e.g., serum, plasma, platelets, red blood cells, and the like), sputum, tissue, cultured cells (e.g., primary cultures, explants, and transformed cells), stool, urine, and the like. In embodiments, a biological sample is blood. In embodiments, a biological sample is a tumor cell. In embodiments, a biological sample is a tumor.

The term “biomarker” refers to an indicator, e.g., a predictive, prognostic, and/or a pharmacodynamic indicator, which can be detected in a biological sample. The biomarker may serve as an indicator of the likelihood a patient will respond to a particular therapeutic treatment or a particular subtype of a disease or disorder, characterized by certain molecular, pathological, histological, and/or clinical features. In embodiments, a biomarker is a cytokine. In embodiments, a biomarker is a gene or a set of genes (i.e., a biomarker gene). Biomarkers include, but are not limited to, polynucleotides (e.g., DNA, and/or RNA), polynucleotide copy number alterations (e.g., DNA copy numbers), polypeptides, or polypeptide and polynucleotide modifications (e.g., posttranslational modifications). In embodiments, the biomarker is LAG3, TIGIT, PD-1, IFNγ, or granzyme B.

The terms an “increased expression level” or “increased level” of gene expression is an expression level of the gene that is higher than the expression level of the gene in a control. The control may be any control known in the art, such as those described herein. In embodiments, an “increased level” of the biomarker gene compared to the control (when the expression level of the biomarker is greater than the corresponding control) is, for example, an increase in the expression level of about 10%, 20%, 30%, 40%, 50%, 60%, 70%, 80%, 90%, 95%, 96%, 97%, 98% or 99% or greater relative to the control. In embodiments, an “increased level” of the biomarker gene is an amount that is statistically significantly greater than the expression level of the control.

The terms a “decreased expression level” or “decreased level” of gene expression is an expression level of the gene that is lower than the expression level of the gene in a control. The control may be any control known in the art, such as those described herein. In embodiments, a “decreased level” of the biomarker gene compared to the control (when the expression level of the biomarker is lower than the corresponding control) is, for example, a decrease in the expression level of about 10%, 20%, 30%, 40%, 50%, 60%, 70%, 80%, 90%, 95%, 96%, 97%, 98% or 99% or greater relative to the control. In embodiments, a “decreased level” of the biomarker gene is an amount that is statistically significantly lower than the expression level of the control.

“Control” is used in accordance with its plain ordinary meaning and refers to an assay, comparison, or experiment in which the subjects or reagents of the experiment are treated as in a parallel experiment except for omission of a procedure, reagent, or variable of the experiment. In embodiments, the control is used as a standard of comparison in evaluating experimental effects. In embodiments, the control is a gene expression level against which another gene expression level (e.g. the gene expression level of a biomarker gene disclosed herein) is compared (e.g., to make a diagnostic (e.g., predictive and/or prognostic) and/or therapeutic determination.

In embodiments, a control is a healthy patient or a healthy population of patients. In embodiments, a healthy patient is a patient that does not have cancer. In embodiments, the control is an average value from population of healthy patients. In embodiments, a control is a pre-assigned value, e.g., a cut-off value which was previously determined to significantly separate a first group of patients (e.g., cancer patients with T cell exhaustion) from a second group of patients (e.g., cancer patients that do not have T cell exhaustion). In embodiments, the cut-off value is the median or mean (preferably median) gene expression level in the reference population. A control can also be obtained from the same individual, e.g., from an earlier-obtained sample, prior to disease, or prior to treatment. One of skill will recognize that controls can be designed for assessment of any number of parameters. In embodiments, a control is a negative control. In embodiments, such as some embodiments relating to detecting the level of expression of a gene/protein or a subset of genes/proteins, a control comprises the average amount of expression (e.g., protein or mRNA) in a population of subjects (e.g., with cancer) or in a healthy or general population. In embodiments, the control comprises an average amount (e.g. amount of expression) in a population in which the number of subjects (n) is 5 or more, 20 or more, 50 or more, 100 or more, 1,000 or more, and the like. One of skill in the art will understand which controls are valuable in a given situation and be able to analyze data based on comparisons to control values.

Biomarker levels may be detected at either the protein (e.g., cytokine) or gene expression level. Proteins expressed by biomarkers can be quantified by immunohistochemistry (IHC), ELISA, or flow cytometry with an antibody that detects the proteins. Biomarker expression can be and quantified by multiple platforms known in the art. Quantifying biomarker (gene) expression can alternatively be referred to as detecting a level of biomarker (gene) expression. Platforms that can be used to quantify biomarker (gene) expression or detect levels of biomarker (gene) expression include quantitative polymerase chain reaction (qPCR), multiplex quantitative polymerase chain reaction (multiplex qPCR), real-time polymerase chain reaction (rtPCR), Nanostring (e.g., an amplification-free technology that measures nucleic acid content by counting molecules directly), RNA-sequencing (using next-generation sequencing (NGS) to reveal the presence and quantity of RNA in a biological sample), or in situ hybridization. There is a range of biomarker expression across as measured by Nanostring. In embodiments, quantitative rtPCR, Nanostring, RNA-sequencing (RNAseq), and in situ hybridization are used to quantitate biomarker gene expression. In embodiments, biomarker expression is quantified by RNAseq. In embodiments, biomarker expression is quantified by multiplex qPCR. In embodiments, biomarker expression is quantified by NanoString. For Nanostring, RNA is extracted from a biological sample and a known quantity of RNA is placed on the Nanostring machine for gene expression detection using gene specific probes. The number of counts of biomarkers within a sample is determined and normalized to a set of housekeeping genes. To determine a threshold for increased or decreased biomarker levels, one skilled in the art could assess biomarker levels in a control group of samples and select the 10^{th}, 20^{th}, 25^{th}, 30^{th}, 40^{th}, 50^{th}, 60^{th}, 70^{th}, 75^{th}, 80^{th} or 90^{th} percentile of biomarker gene expression. In embodiments, the increased or decreased expression of biomarkers may be determined by calculating the H-score for the expression of the biomarkers. Thus, the increased or decreased expression of biomarkers may have an H-score. As used herein, an “H-score” or “Histoscore” is a numerical value determined by a semi-quantitative method commonly known for immunohistochemically evaluating protein expression in tumor samples.

The disclosure provides methods of treating a patient having deficient Th1 activity by administering to the patient an ITK inhibitor at an effective amount to increase Th1 activity. In embodiments, the patient has increased Th2 activity. In embodiments, the patient has increased levels of pro-inflammatory cytokines. In embodiments, the patient has increased level of a pro-inflammatory cytokine selected from the group consisting of IL-4, IL-5, IL10, IL-13, and a combination of two or more thereof. In embodiments, the patient has increased level of a pro-inflammatory cytokine selected from the group consisting of IL-4, IL-5, IL10, IL-13, IL-17, and a combination of two or more thereof. In embodiments, an effective amount to increase Th1 activity is an amount of an ITK inhibitor that increases the number of Th1⁺ T cells; increases the ratio of Th1¹ T cells to Th2¹ T cells; increases the ratio of IFNγ¹CD4¹ T cells to IL-4¹CD4¹ T cells; increases IFNγ production; increases CD8+ cytotoxic lymphocytes; inhibits IL-4 production; inhibits IL-5 production; inhibits IL-13 production; decreases Th2+ cells; decreases Th17+ T cells; decreases eosinophils; or a combination of two or more of the foregoing. In embodiments, an effective amount to increase Th1 activity is an amount of an ITK inhibitor that increases the number of Th1⁺ T cells; increases the ratio of Th1⁺ T cells to Th2⁺ T cells; increases the ratio of IFNγ⁺CD4⁺ T cells to IL-4⁺CD4⁺ T cells; increases IFNγ production; increases CD8⁺ cytotoxic lymphocytes; inhibits IL-4 production; decreases Th17⁺ T cells; decreases eosinophils; or a combination of two or more of the foregoing. In embodiments, an effective amount to increase Th1 activity is an amount of an ITK inhibitor that increases the number of Th1⁺ T cells; increases the ratio of Th1⁺ T cells to Th2⁺ T cells; increases the ratio of IFNγ⁺CD4⁺ T cells to IL-4⁺CD4⁺ T cells; increases IFNγ production; increases CD8⁺ cytotoxic lymphocytes; inhibits IL-4 production; decreases Th17⁺ T cells; decreases eosinophils; inhibits Th2; or inhibits the differentiation of naïve CD4 cells into Th2 cells; or a combination of two or more of the foregoing. In embodiments, an effective amount to increase Th1 activity is an amount of an ITK inhibitor that increases the number of Th1⁺ T cells. In embodiments, an effective amount to increase Th1 activity is an amount of an ITK inhibitor that increases the ratio of Th1⁺ T cells to Th2⁺ T cells. In embodiments, an effective amount to increase Th1 activity is an amount of an ITK inhibitor that increases the ratio of IFNγ⁺CD4⁺ T cells to IL-4⁺CD4⁺ T cells. In embodiments, an effective amount to increase Th1 activity is an amount of an ITK inhibitor that increases IFN-γ production. In embodiments, an effective amount to increase Th1 activity is an amount of an ITK inhibitor that increases CD8+ cytotoxic lymphocytes. In embodiments, an effective amount to increase Th1 activity is an amount of an ITK inhibitor that inhibits IL-4 production. In embodiments, an effective amount to increase Th1 activity is an amount of an ITK inhibitor that inhibits IL-5 production. In embodiments, an effective amount to increase Th1 activity is an amount of an ITK inhibitor that inhibits IL-13 production. In embodiments, an effective amount to increase Th1 activity is an amount of an ITK inhibitor that inhibits cytokines secreted by Th2+ cells. In embodiments, an effective amount to increase Th1 activity is an amount of an ITK inhibitor that decreases Th2+ cells. In embodiments, an effective amount to increase Th1 activity is an amount of an ITK inhibitor that decreases Th17+ T cells. In embodiments, an effective amount to increase Th1 activity is an amount of an ITK inhibitor that decreases eosinophils. In embodiments, an effective amount to increase Th1 activity is an amount of an ITK inhibitor that blocks Th2 or that blocks the differentiation of naïve CD4 cells into Th2 cells. In embodiments, an effective amount to increase Th1 activity is an amount of an ITK inhibitor that inhibits Th2. In embodiments, an effective amount to increase Th1 activity is an amount of an ITK inhibitor that inhibits the differentiation of naïve CD4 cells into Th2 cells. In embodiments, an effective amount to increase Th1 activity is from about 0.5 mmole to about 2 mmole of the ITK inhibitor per day. In embodiments, “deficient Th1 activity” is decreased Th1 activity relative to a control. In embodiments, the control is a healthy patient or a population of healthy patients.

The disclosure provides methods of treating a patient having deficient Th1 activity by: (i) measuring an increased level of Th2 activity, relative to a control, in a biological sample from the patient, and (ii) administering to the patient an ITK inhibitor at an effective amount to increase Th1 activity. The disclosure provides methods of treating a patient having deficient Th1 activity by: (i) measuring increased level of a cytokine selected from the group consisting of IL-4, IL-5, IL10, IL-13, IL-17, and a combination of two or more thereof, relative to a control, in a biological sample from the patient, and (ii) administering to the patient an ITK inhibitor at an effective amount to increase Th1 activity. The disclosure provides methods of treating a patient having deficient Th1 activity by: (i) measuring an increased level of IL-4, relative to a control, in a biological sample from the patient, and (ii) administering to the patient an ITK inhibitor at an effective amount to increase Th1 activity. The disclosure provides methods of treating a patient having deficient Th1 activity by: (i) measuring an increased level of IL-5, relative to a control, in a biological sample from the patient, and (ii) administering to the patient an ITK inhibitor at an effective amount to increase Th1 activity. The disclosure provides methods of treating a patient having deficient Th1 activity by: (i) measuring an increased level of IL10, relative to a control, in a biological sample from the patient, and (ii) administering to the patient an ITK inhibitor at an effective amount to increase Th1 activity. The disclosure provides methods of treating a patient having deficient Th1 activity by: (i) measuring an increased level of IL-13, relative to a control, in a biological sample from the patient, and (ii) administering to the patient an ITK inhibitor at an effective amount to increase Th1 activity. The disclosure provides methods of treating a patient having deficient Th1 activity by: (i) measuring an increased level of IL-17, relative to a control, in a biological sample from the patient, and (ii) administering to the patient an ITK inhibitor at an effective amount to increase Th1 activity. In embodiments, the patient has increased Th2 activity. In embodiments, the patient has increased levels of pro-inflammatory cytokines. In embodiments, the patient has increased level of a pro-inflammatory cytokine selected from the group consisting of IL-4, IL-5, IL10, IL-13, and a combination of two or more thereof. In embodiments, an effective amount to increase Th1 activity is an amount of an ITK inhibitor that increases the number of Th1⁺ T cells; increases the ratio of Th1⁺ T cells to Th2⁺ T cells; increases the ratio of IFNγ⁺CD4⁺ T cells to IL-4⁺CD4⁺ T cells; increases IFNγ production; increases CD8⁺ cytotoxic lymphocytes; inhibits IL-4 production; decreases Th17⁺ T cells; decreases eosinophils; inhibits Th2; or inhibits the differentiation of naïve CD4 cells into Th2 cells; or a combination of two or more of the foregoing. In embodiments, an effective amount to increase Th1 activity is an amount of an ITK inhibitor that inhibits Th2 or that inhibits the differentiation of naïve CD4 cells into Th2 cells. In embodiments, an effective amount to increase Th1 activity is an amount of an ITK inhibitor that inhibits Th2. In embodiments, an effective amount to increase Th1 activity is an amount of an ITK inhibitor that inhibits the differentiation of naïve CD4 cells into Th2 cells.

In embodiments, the disclosure provides methods of treating a patient having deficient Th1-type cytokines by administering to the patient an ITK inhibitor at an effective amount to increase Th1 activity. In embodiments, the disclosure provides methods of treating a patient having increased Th2 activity by administering to the patient an ITK inhibitor at an effective amount to increase Th1 activity. In embodiments, the disclosure provides methods of treating a patient having deficient Th1 activity and increased Th2 activity by administering to the patient an ITK inhibitor at an effective amount to increase Th1 activity. In embodiments, the disclosure provides methods of treating a patient having increased levels of a pro-inflammatory cytokine by administering to the patient an ITK inhibitor at an effective amount to increase Th1 activity. In embodiments, the disclosure provides methods of treating a patient having deficient Th1 activity and increased levels of a pro-inflammatory cytokine by administering to the patient an ITK inhibitor at an effective amount to increase Th1 activity. In embodiments, “deficient Th1-type cytokines” refers to decreased Th1 levels of Th1-type cytokines. In embodiments, a Th1-type cytokines include IFNγ, IL-10, IL-2, IL-12, TNF-α, TNF-γ, and granulocyte-macrophage colony-stimulating factor (GMCS). In embodiments, “increased Th2 activity” is increased Th2 activity relative to a control. In embodiments, “increased levels of a pro-inflammatory cytokine” is relative to a control. In embodiments, “a pro-inflammatory cytokine” is IL-4, IL-5, IL10, IL-13, or a combination of two or more thereof. In embodiments, “a pro-inflammatory cytokine” is IL-4, IL-5, IL10, IL-13, IL-17, or a combination of two or more thereof. In embodiments, the control is a healthy patient or a population of healthy patients. In embodiments, the disclosure provides methods of treating a patient having deficient Th1-type cytokines by administering to the patient an ITK inhibitor at an effective amount to increase Th1 activity. In embodiments, the disclosure provides methods of treating a patient having deficient Th1-type cytokines by: (i) measuring decreased levels of Th1, IFNγ, IL-β, IL-2, IL-12, TNF-α, TNF-γ, GMCS, or a combination of two or more thereof, relative to a control, in a biological sample from the patient; and (ii) administering to the patient an ITK inhibitor at an effective amount to increase Th1 activity. In embodiments, the disclosure provides methods of treating a patient having deficient Th1-type cytokines by: (i) measuring increased levels of IL-4, IL-5, IL10, IL-13, IL-17, or a combination of two or more thereof, relative to a control, in a biological sample from the patient; and (ii) administering to the patient an ITK inhibitor at an effective amount to increase Th1 activity. In embodiments, the disclosure provides methods of treating a patient having increased Th2 activity by administering to the patient an ITK inhibitor at an effective amount to increase Th1 activity. In embodiments, the disclosure provides methods of treating a patient having increased Th2 activity by: (i) measuring an increased level of Th2, relative to a control, in a biological sample obtained from the patient, and (ii) administering to the patient an ITK inhibitor at an effective amount to increase Th1 activity. In embodiments, the disclosure provides methods of treating a patient having deficient Th1 activity and increased Th2 activity by administering to the patient an ITK inhibitor at an effective amount to increase Th1 activity. In embodiments, the disclosure provides methods of treating a patient having increased levels of a pro-inflammatory cytokine by administering to the patient an ITK inhibitor at an effective amount to increase Th1 activity. In embodiments, the disclosure provides methods of treating a patient having deficient Th1 activity and increased levels of a pro-inflammatory cytokine by administering to the patient an ITK inhibitor at an effective amount to increase Th1 activity. In embodiments, “deficient Th1-type cytokines” refers to decreased Th1 levels of Th1-type cytokines. In embodiments, a Th1-type cytokines include IFNγ, IL-10, IL-2, IL-12, TNF-α, TNF-γ, and granulocyte-macrophage colony-stimulating factor (GMCS). In embodiments, “increased Th2 activity” is increased Th2 activity relative to a control. In embodiments, “increased levels of a pro-inflammatory cytokine” is relative to a control. In embodiments, “a pro-inflammatory cytokine” is IL-4, IL-5, IL10, IL-13, IL-17, or a combination of two or more thereof. In embodiments, the control is a healthy patient or a population of healthy patients.

“Th1 activity” refers to the number of Th1 cells and/or to the activity of the Th1 pathway. The Th-1 pathway refers to Th1-type cytokines that activate the immune system to suppress tumors. Th1-type cytokines include IFNγ, IL-1β, IL-2, IL-12, TNF-α, TNF-γ, GMCS, or a combination of two or more thereof. Thus, “deficient Th1 activity” includes reduced number or reduced expression of Th1-type cytokines.

“Th2 activity” refers to the number of Th2 cells and/or the activity of the Th2 pathway. The Th2 pathway refers to Th2-type cytokines that are related to tumor growth or metastasis. Th2-type cytokines include IL-4, IL-5, IL-10, IL-13, IL-17, or a combination of two or more thereof.

T cell exhaustion is a state of T cells in which they lose their effector functions and proliferation capacity. Provided herein is a method of reversing T cell exhaustion in a patient in need thereof comprising administering to the patient an effective amount of an ITK inhibitor. In embodiments, reversing T cell exhaustion is treating a patient that has T cell exhaustion. In embodiments, reversing T cell exhaustion is overcoming T cell exhaustion. In embodiments, the method of reversing T cell exhaustion in a patient in need thereof comprises: (i) measuring an increased level of LAG3, an increased level of TIGIT, an increased level of PD-i, or a combination of two or more thereof, relative to a control, in a biological sample obtained from the patient; and (ii) administering to the patient an effective amount of an ITK inhibitor. In embodiments, the method of reversing T cell exhaustion in a patient in need thereof comprises: (i) measuring an increased level of LAG3, relative to a control, in a biological sample obtained from the patient; and (ii) administering to the patient an effective amount of an ITK inhibitor. In embodiments, the method of reversing T cell exhaustion in a patient in need thereof comprises: (i) measuring an increased level of TIGIT, in a biological sample obtained from the patient; and (ii) administering to the patient an effective amount of an ITK inhibitor. In embodiments, the method of reversing T cell exhaustion in a patient in need thereof comprises: (i) measuring an increased level of PD-1, relative to a control, in a biological sample obtained from the patient; and (ii) administering to the patient an effective amount of an ITK inhibitor. In embodiments, the method of reversing T cell exhaustion in a patient in need thereof comprises: (i) measuring a decreased level of IFNγ, a decreased level of granzyme B, or a combination thereof, relative to a control, in a biological sample obtained from the patient; and (ii) administering to the patient an effective amount of an ITK inhibitor. In embodiments, the method of reversing T cell exhaustion in a patient in need thereof comprises: (i) measuring a decreased level of IFNγ, relative to a control, in a biological sample obtained from the patient; and (ii) administering to the patient an effective amount of an ITK inhibitor. In embodiments, the method of reversing T cell exhaustion in a patient in need thereof comprises: (i) measuring a decreased level of granzyme B, relative to a control, in a biological sample obtained from the patient; and (ii) administering to the patient an effective amount of an ITK inhibitor. In embodiments, the method of reversing T cell exhaustion in a patient in need thereof comprises: (i) measuring an increased level of LAG3, an increased level of TIGIT, an increased level of PD-1, a decreased level of IFNγ, a decreased level of granzyme B, or a combination of two or more thereof, relative to a control, in a biological sample obtained from the patient; and (ii) administering to the patient an effective amount of an ITK inhibitor. In embodiments, a patient is identified as having T cell exhaustion when a biological sample obtained from the patient has, relative to a control, (i) an increased level of LAG3, (ii) an increased level of TIGIT, (iii) an increased level of PD-1, (iv) a decreased level of IFNγ, (v) a decreased level of granzyme B, or (vi) a combination of two or more of (i)-(v). In embodiments, the T cell is a CD4 T cell. In embodiments, the patient has cancer. In embodiments, the method which comprises administering the ITK inhibitor increases levels of IFNγ; increases levels of granzyme B; or a combination thereof, relative to a control or relative to the levels of IFNγ and/or granzyme B prior to administration of the ITK inhibitor. In embodiments, the method which comprises administering the ITK inhibitor decreases the level of LAG3, decreases the level of TIGIT, decreases the level of PD-1, or the combination of two or more thereof, relative to a control or relative to the level of LAG3, TIGIT, PD-1, or the combination of two or more thereof, prior to administration of the ITK inhibitor. In embodiments, the ITK inhibitor is a compound of Formula (I) or a pharmaceutically acceptable salt thereof. In embodiments, the ITK inhibitor is a compound of Formula (TT) or a pharmaceutically acceptable salt thereof. In embodiments, the ITK inhibitor is a compound of Formula (A) or a pharmaceutically acceptable salt thereof. In embodiments, the ITK inhibitor is the free base form of the compound of Formula (A). In embodiments, the ITK inhibitor is a compound of any one of Formula (1) to Formula (34) or a pharmaceutically acceptable salt thereof.

The disclosure provides methods of treating cancer in a subject in need thereof by administering to the subject an effective amount of an ITK inhibitor or a pharmaceutically acceptable salt thereof. The disclosure provides methods of treating cancer in a subject in need thereof by administering to the subject an effective amount of a pharmaceutical composition comprising an ITK inhibitor or a pharmaceutically acceptable salt thereof, and a pharmaceutically acceptable excipient. In embodiments, the ITK inhibitor is a compound of Formula (I) or a pharmaceutically acceptable salt thereof. In embodiments, the ITK inhibitor is a compound of Formula (II) or a pharmaceutically acceptable salt thereof. In embodiments, the ITK inhibitor is a compound of Formula (A) or a pharmaceutically acceptable salt thereof. In embodiments, the ITK inhibitor is the free base form of the compound of Formula (A). In embodiments, the ITK inhibitor is a compound of any one of Formula (1) to Formula (34) or a pharmaceutically acceptable salt thereof.

Provided herein is a method of treating cancer in a patient in need thereof comprising administering to the patient an effective amount of an ITK inhibitor; wherein the patient has T cell exhaustion. In embodiments, the patient has an increased level of LAG3, an increased level of TIGIT, an increased level of PD-1, or a combination of two or more thereof, relative to a control, in a biological sample obtained from the patient. In embodiments, the patient has a decreased level of IFNγ, a decreased level of granzyme B, or a combination thereof, relative to a control, in a biological sample obtained from the patient. In embodiments, the patient has an increased level of LAG3, an increased level of TIGIT, an increased level of PD-1, a decreased level of IFNγ, a decreased level of granzyme B, or a combination of two or more thereof, relative to a control, in a biological sample obtained from the patient. In embodiments, a patient is identified as having T cell exhaustion when a biological sample obtained from the patient has, relative to a control, (i) an increased level of LAG3, (ii) an increased level of TIGIT, (iii) an increased level of PD-1, (iv) a decreased level of IFNγ, (v) a decreased level of granzyme B, or (vi) a combination of two or more of (i)-(v). In embodiments, the method of treating cancer increases levels of IFNγ; increases levels of granzyme B; or a combination thereof, relative to a control or relative to the levels of IFNγ and/or granzyme B prior to administration of the ITK inhibitor. In embodiments, the method of treating cancer decreases the level of LAG3, the level of TIGIT, the level of PD-1, or a combination of two or more thereof, relative to a control or relative to the level of LAG3, TIGIT, PD-1, or the combination of two or more thereof, prior to administration of the ITK inhibitor. In embodiments, the ITK inhibitor is a compound of Formula (I) or a pharmaceutically acceptable salt thereof. In embodiments, the ITK inhibitor is a compound of Formula (II) or a pharmaceutically acceptable salt thereof. In embodiments, the ITK inhibitor is a compound of Formula (A) or a pharmaceutically acceptable salt thereof. In embodiments, the ITK inhibitor is the free base form of the compound of Formula (A). In embodiments, the ITK inhibitor is a compound of any one of Formula (1) to Formula (34) or a pharmaceutically acceptable salt thereof.

Provided herein is a method of treating cancer in a patient in need thereof comprising (i) measuring an increased level of LAG3, an increased level of TIGIT, an increased level of PD-1, or a combination of two or more thereof, relative to a control, in a biological sample obtained from the patient; and (ii) administering to the patient an effective amount of an ITK inhibitor. Provided herein is a method of treating cancer in a patient in need thereof, the method comprising (i) measuring a decreased level of IFNγ, a decreased level of granzyme B, or a combination thereof, relative to a control, in a biological sample obtained from the patient; and (ii) administering to the patient an effective amount of an ITK inhibitor. Provided herein is a method of treating cancer in a patient in need thereof comprising (i) measuring an increased level of LAG3, an increased level of TIGIT, an increased level of PD-1, a decreased level of IFNγ, a decreased level of granzyme B, or a combination of two or more thereof, relative to a control, in a biological sample obtained from the patient; and (ii) administering to the patient an effective amount of an ITK inhibitor. In embodiments, a patient is identified as having T cell exhaustion when a biological sample obtained from the patient has, relative to a control, (i) an increased level of LAG3, (ii) an increased level of TIGIT, (iii) an increased level of PD-1, (iv) a decreased level of IFNγ, (v) a decreased level of granzyme B, or (vi) a combination of two or more of (i)-(v). In embodiments, the method of treating cancer decreases the level of LAG3, decreases the level of TIGIT, decreases the level of PD-1, increases the level of IFNγ, increases the level of granzyme B, or a combination of two or more thereof, relative to a control or relative to the level of LAG3, TIGIT, PD-1, IFNγ, granzyme B, or the combination of two or more thereof, prior to administration of the ITK inhibitor. In embodiments, the ITK inhibitor is a compound of Formula (I) or a pharmaceutically acceptable salt thereof. In embodiments, the ITK inhibitor is a compound of Formula (II) or a pharmaceutically acceptable salt thereof. In embodiments, the ITK inhibitor is a compound of Formula (A) or a pharmaceutically acceptable salt thereof. In embodiments, the ITK inhibitor is the free base form of the compound of Formula (A). In embodiments, the ITK inhibitor is a compound of any one of Formula (1) to Formula (34) or a pharmaceutically acceptable salt thereof.

In embodiments, the cancer is lymphoma. In embodiments, the lymphoma is T-cell lymphoma. In embodiments, the lymphoma is peripheral T-cell lymphoma. In embodiments, the lymphoma is peripheral T-cell lymphoma not otherwise specified. In embodiments, the lymphoma is cutaneous T-cell lymphoma. In embodiments, the lymphoma is cutaneous T-cell lymphoma not otherwise specified. In embodiments, the lymphoma is angioimmunoblastic T cell lymphoma. In embodiments, the lymphoma is NK T cell lymphoma. In embodiments, the cancer is a solid tumor. In embodiments, the cancer is lung cancer, colorectal cancer, pancreatic cancer, prostate cancer, breast cancer, gastric cancer, or head and neck cancer. In embodiments, the cancer is lung cancer. In embodiments, the cancer is colorectal cancer. In embodiments, the cancer is pancreatic cancer. In embodiments, the cancer is prostate cancer. In embodiments, the cancer is breast cancer. In embodiments, the cancer is gastric cancer. In embodiments, the cancer is head and neck cancer. In embodiments, the cancer is leukemia. In embodiments, the cancer is T cell leukemia. In embodiments, the cancer is T cell lymphoma, T cell leukemia, lung cancer, colorectal cancer, pancreatic cancer, prostate cancer, breast cancer, gastric cancer, or head and neck cancer.

In embodiments, the cancer is relapsed/refractory cancer. In embodiments, the cancer is relapsed/refractory leukemia. In embodiments, the cancer is relapsed/refractory T cell leukemia. In embodiments, the cancer is relapsed/refractory lymphoma. In embodiments, the lymphoma is relapsed/refractory T-cell lymphoma. In embodiments, the lymphoma is relapsed/refractory peripheral T-cell lymphoma. In embodiments, the lymphoma is relapsed/refractory peripheral T-cell lymphoma not otherwise specified. In embodiments, the lymphoma is relapsed/refractory cutaneous T-cell lymphoma. In embodiments, the cancer is a relapsed/refractory solid tumor. In embodiments, the relapsed/refractory cancer is lung cancer, colorectal cancer, pancreatic cancer, prostate cancer, breast cancer, gastric cancer, or head and neck cancer. In embodiments, the cancer is relapsed/refractory lung cancer. In embodiments, the cancer is relapsed/refractory colorectal cancer. In embodiments, the cancer is relapsed/refractory pancreatic cancer. In embodiments, the cancer is relapsed/refractory prostate cancer. In embodiments, the cancer is relapsed/refractory breast cancer. In embodiments, the cancer is relapsed/refractory gastric cancer. In embodiments, the cancer is relapsed/refractory head and neck cancer. In embodiments, the relapsed/refractory cancer is T cell lymphoma, T cell leukemia, lung cancer, colorectal cancer, pancreatic cancer, prostate cancer, breast cancer, gastric cancer, or head and neck cancer.

The term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma (including angioimmunoblastic T cell lymphoma and peripheral T cell lymphoma not otherwise specified), anaplastic large cell lymphoma, mycosis fungoides, NK T cell lymphoma and precursor T-lymphoblastic lymphoma.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The disclosure provides methods of treating an autoimmune disease in a subject in need thereof by administering to the subject an effective amount of an ITK inhibitor or a pharmaceutically acceptable salt thereof. The disclosure provides methods of treating an autoimmune disease in a subject in need thereof by administering to the subject an effective amount of a pharmaceutical composition comprising an ITK inhibitor or a pharmaceutically acceptable salt thereof, and a pharmaceutically acceptable excipient. In embodiments, the ITK inhibitor is a compound of Formula (1) or a pharmaceutically acceptable salt thereof. In embodiments, the ITK inhibitor is a compound of Formula (II) or a pharmaceutically acceptable salt thereof. In embodiments, the ITK inhibitor is a compound of Formula (A) or a pharmaceutically acceptable salt thereof. In embodiments, the ITK inhibitor is the free base form of the compound of Formula (A). In embodiments, the ITK inhibitor is a compound of any one of Formula (1) to Formula (34) or a pharmaceutically acceptable salt thereof. In embodiments, the autoimmune disease is autoimmune lymphoproliferative disease (autoimmune lymphoproliferative syndrome), colitis, or systemic lupus erythematosus. In embodiments, the autoimmune disease is autoimmune lymphoproliferative disease, ulcerative colitis, or systemic lupus erythematosus. In embodiments, the autoimmune disease is autoimmune lymphoproliferative disease. In embodiments, the autoimmune disease is colitis. In embodiments, the autoimmune disease is ulcerative colitis. In embodiments, the autoimmune disease is inflammatory bowel disease. In embodiments, the autoimmune disease is systemic lupus erythematosus.

In embodiments, the autoimmune disease is acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticaria, axonal or neuronal neuropathies, balo disease, Behcet's disease, bullous pemphigoid, cardiomyopathy, Castleman disease, celiac disease, Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogans syndrome, cold agglutinin disease, congenital heart block, coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, evans syndrome, fibromyalgia, fibrosing alveolitis, giant cell arteritis (temporal arteritis), giant cell myocarditis, glomerulonephritis, Goodpasture's syndrome, granulomatosis with polyangiitis (GPA) (formerly called Wegener's Granulomatosis), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenic purpura (ITP), IgA nephropathy, IgG4-related sclerosing disease, immunoregulatory lipoproteins, inclusion body myositis, interstitial cystitis, juvenile arthritis, Type 1 diabetes, juvenile myositis, Kawasaki syndrome, Lambert-Eaton syndrome, leukocytoclastic vasculitis, lichen planus, lichen sclerosus, ligneous conjunctivitis, linear IgA disease (LAD), lupus (SLE), Lyme disease, chronic, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Tumer syndrome, pars planitis (peripheral uveitis), pemphigus, peripheral neuropathy, perivenous encephalomyelitis, pernicious anemia, POEMS syndrome, Ppolyarteritis nodosa, Type I, II, & III autoimmune polyglandular syndromes, polymyalgia rheumatica, polymyositis, postmyocardial infarction syndrome, postpericardiotomy syndrome, progesterone dermatitis, primary biliary cirrhosis, primary sclerosing cholangitis, psoriatic arthritis, idiopathic pulmonary fibrosis, pyoderma gangrenosum, pure red cell aplasia, Raynauds phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/giant cell arteritis, thrombocytopenic purpura (TTP), Tolosa-Hunt syndrome, transverse myelitis, ulcerative colitis, undifferentiated connective tissue disease (UCTD), uveitis, vasculitis, vesiculobullous dermatosis, vitiligo, or Wegener's granulomatosis (i.e., Granulomatosis with polyangiitis (GPA).

The disclosure provides methods of treating an allergy in a subject in need thereof by administering to the subject an effective amount of an ITK inhibitor or a pharmaceutically acceptable salt thereof. The disclosure provides methods of treating an allergy in a subject in need thereof by administering to the subject an effective amount of a pharmaceutical composition comprising an ITK inhibitor or a pharmaceutically acceptable salt thereof, and a pharmaceutically acceptable excipient. In embodiments, the ITK inhibitor is a compound of Formula (I) or a pharmaceutically acceptable salt thereof. In embodiments, the ITK inhibitor is a compound of Formula (II) or a pharmaceutically acceptable salt thereof. In embodiments, the ITK inhibitor is a compound of Formula (A) or a pharmaceutically acceptable salt thereof. In embodiments, the ITK inhibitor is the free base form of the compound of Formula (A). In embodiments, the ITK inhibitor is a compound of any one of Formula (1) to Formula (34) or a pharmaceutically acceptable salt thereof. In embodiments, the allergy is an IgE-mediated allergy. In embodiments, the allergy is caused by Th2 cells. In embodiments, the allergy is Th2 cell-mediated inflammation. In embodiments, the allergy is asthma, rhinitis, dermatitis, or psoriasis. In embodiments, the allergy is allergic asthma, allergic rhinitis, atopic dermatitis, allergic dermatitis, or psoriasis. In embodiments, the allergy is asthma, rhinitis, or dermatitis. In embodiments, the allergy is allergic asthma, allergic rhinitis, atopic dermatitis, or allergic dermatitis. In embodiments, the allergy is allergic asthma, allergic rhinitis, or allergic dermatitis. In embodiments, the allergy is allergic asthma. In embodiments, the allergy is asthma. In embodiments, the allergy is allergic rhinitis. In embodiments, the allergy is rhinitis. In embodiments, the allergy is atopic dermatitis. In embodiments, the allergy is allergic dermatitis. In embodiments, the allergy is dermatitis. In embodiments, the allergy is psoriasis.

The disclosure provides methods of treating an Th2/ITK-mediated disease in a subject in need thereof by administering to the subject an effective amount of an ITK inhibitor or a pharmaceutically acceptable salt thereof. The disclosure provides methods of treating a Th2/ITK-mediated disease in a subject in need thereof by measuring a decreased level of Th1⁺ T cells, a decreased ratio of Th1⁺ T cells to Th2⁺ T cells; a decreased ratio of IFNγ⁺CD4⁺ T cells to IL-4⁺CD4⁺ T cells; a decreased level of IFNγ; a decreased level CD8⁺ cytotoxic lymphocytes; an increased level of Th2⁺ cells; an increased level of IL-4; an increased level of IL-5; an increased level of IL-10; an increased level of IL-13; an increased level of IL-17; an increased level of Th17+ T cells; an increased level of eosinophils; a decreased level of IL-10, a decreased level of IL-2, a decreased level of IL-12, a decreased level of TNF-α, a decreased level of TNF-γ, a decreased level of GMCS, or a combination of two or more thereof, relative to a control, in a biological sample obtained from the subject, and administering to the subject an effective amount of an ITK inhibitor or a pharmaceutically acceptable salt thereof. The disclosure provides methods of treating an Th2/ITK-mediated disease in a subject in need thereof by measuring an increased level of Th2+ cells; an increased level of IL-4; an increased level of IL-5; an increased level of IL-10; an increased level of IL-13; an increased level of IL-17; or a combination of two or more thereof, relative to a control, in a biological sample obtained from the subject, and administering to the subject an effective amount of an ITK inhibitor or a pharmaceutically acceptable salt thereof. In embodiments, the method of treating an Th2/ITK-mediated disease in a subject in need thereof comprises measuring an increased level of Th2+ cells, relative to a control, in a biological sample obtained from the subject, and administering to the subject an effective amount of an ITK inhibitor or a pharmaceutically acceptable salt thereof. In embodiments, the method of treating an Th2/ITK-mediated disease in a subject in need thereof comprises measuring an increased level of IL-4; an increased level of IL-5; an increased level of IL-10; an increased level of IL-13; an increased level of IL-17; or a combination of two or more thereof, relative to a control, in a biological sample obtained from the subject, and administering to the subject an effective amount of an ITK inhibitor or a pharmaceutically acceptable salt thereof. In embodiments, the method of treating an Th2/ITK-mediated disease in a subject in need thereof comprises measuring an increased level of IL-4, relative to a control, in a biological sample obtained from the subject, and administering to the subject an effective amount of an ITK inhibitor or a pharmaceutically acceptable salt thereof. In embodiments, the method of treating an Th2/ITK-mediated disease in a subject in need thereof comprises measuring an increased level of IL-5, relative to a control, in a biological sample obtained from the subject, and administering to the subject an effective amount of an ITK inhibitor or a pharmaceutically acceptable salt thereof. In embodiments, the method of treating an Th2/ITK-mediated disease in a subject in need thereof comprises measuring an increased level of IL-10, relative to a control, in a biological sample obtained from the subject, and administering to the subject an effective amount of an ITK inhibitor or a pharmaceutically acceptable salt thereof. In embodiments, the method of treating an Th2/ITK-mediated disease in a subject in need thereof comprises measuring an increased level of IL-13, relative to a control, in a biological sample obtained from the subject, and administering to the subject an effective amount of an ITK inhibitor or a pharmaceutically acceptable salt thereof. In embodiments, the method of treating an Th2/ITK-mediated disease in a subject in need thereof comprises measuring an increased level of IL-17, relative to a control, in a biological sample obtained from the subject, and administering to the subject an effective amount of an ITK inhibitor or a pharmaceutically acceptable salt thereof. The disclosure provides methods of treating a Th2/ITK-mediated disease in a subject in need thereof by measuring an increased level of Th2⁺ cells, relative to a control, in a biological sample obtained from the subject, and administering to the subject an effective amount of an ITK inhibitor or a pharmaceutically acceptable salt thereof. The disclosure provides methods of treating a Th2/ITK-mediated disease in a subject in need thereof by measuring an increased level of IL-4; an increased level of IL-5; an increased level of IL-10; an increased level of IL-13; an increased level of IL-17; or a combination of two or more thereof, relative to a control, in a biological sample obtained from the subject, and administering to the subject an effective amount of an ITK inhibitor or a pharmaceutically acceptable salt thereof. The disclosure provides methods of treating an Th2/ITK-mediated disease in a subject in need thereof by administering to the subject an effective amount of a pharmaceutical composition comprising an ITK inhibitor or a pharmaceutically acceptable salt thereof, and a pharmaceutically acceptable excipient. In embodiments, the ITK inhibitor is a compound of Formula (I) or a pharmaceutically acceptable salt thereof. In embodiments, the ITK inhibitor is a compound of Formula (II) or a pharmaceutically acceptable salt thereof. In embodiments, the ITK inhibitor is a compound of Formula (A) or a pharmaceutically acceptable salt thereof. In embodiments, the ITK inhibitor is the free base form of the compound of Formula (A). In embodiments, the ITK inhibitor is a compound of any one of Formula (1) to Formula (34) or a pharmaceutically acceptable salt thereof. In embodiments, the Th2/ITK-mediated disease is an autoimmune disease or an allergy. In embodiments, the Th2/ITK-mediated disease is an autoimmune disease. In embodiments, the Th2/ITK-mediated disease is an allergy. In embodiments, the Th2/ITK-mediated disease is atopic dermatitis, asthma, rhinitis, conjunctivitis, psoriasis, a fibrotic disease, psoriatic arthritis, vasculitis, autoimmune lymphoproliferative syndrome, chronic obstructive pulmonary disease, an eosinophilic disease, a mast cell disease, or human immunodeficiency viral disease.

In embodiments of the methods described herein, the Th2/ITK-mediated disease is atopic dermatitis, asthma, rhinitis, conjunctivitis, psoriasis, scleroderma, pulmonary fibrosis, cirrhosis, retroperitoneal fibrosis, psoriatic arthritis, vasculitis, autoimmune lymphoproliferative syndrome, chronic obstructive pulmonary disease, an eosinophilic disease, a mast cell disease, or human immunodeficiency viral disease. In embodiments, the Th2/ITK-mediated disease is atopic dermatitis, asthma, rhinitis, conjunctivitis, psoriasis, scleroderma, pulmonary fibrosis, cirrhosis, retroperitoneal fibrosis, psoriatic arthritis, vasculitis, autoimmune lymphoproliferative syndrome, chronic obstructive pulmonary disease, esophagitis, mastocytosis, mast cell activation syndrome, hereditary alpha tryptasemia, or human immunodeficiency viral disease. In embodiments, the Th2/ITK-mediated disease is atopic dermatitis, asthma, or pulmonary fibrosis. In embodiments, the Th2/ITK-mediated disease is atopic dermatitis, asthma, or idiopathic pulmonary fibrosis. In embodiments, the Th2/ITK-mediated disease is atopic dermatitis, asthma, rhinitis, or conjunctivitis. In embodiments, the Th2/ITK-mediated disease is scleroderma, pulmonary fibrosis, cirrhosis, or retroperitoneal fibrosis. In embodiments, the Th2/ITK-mediated disease is atopic dermatitis, asthma, rhinitis, or conjunctivitis. In embodiments, the Th2/ITK-mediated disease is scleroderma, idiopathic pulmonary fibrosis, cirrhosis, or retroperitoneal fibrosis. In embodiments, the Th2/ITK-mediated disease is autoimmune lymphoproliferative syndrome, chronic obstructive pulmonary disease, an eosinophilic disease, a mast cell disease, or human immunodeficiency viral disease. In embodiments, the Th2/ITK-mediated disease is autoimmune lymphoproliferative syndrome, chronic obstructive pulmonary disease, esophagitis, mastocytosis, mast cell activation syndrome, hereditary alpha tryptasemia, or human immunodeficiency viral disease. In embodiments, the Th2/ITK-mediated disease is psoriatic arthritis or vasculitis.

In embodiments, the Th2/ITK-mediated disease is atopic dermatitis. In embodiments, the Th2/ITK-mediated disease is asthma. In embodiments, the Th2/ITK-mediated disease is rhinitis. In embodiments, the Th2/ITK-mediated disease is conjunctivitis. In embodiments, the Th2/ITK-mediated disease is psoriasis. In embodiments, the Th2/ITK-mediated disease is a fibrotic disease. In embodiments, the fibrotic disease is scleroderma, pulmonary fibrosis, cirrhosis, or retroperitoneal fibrosis. In embodiments, the fibrotic disease is scleroderma, idiopathic pulmonary fibrosis, cirrhosis, or retroperitoneal fibrosis. In embodiments, the Th2/ITK-mediated disease is scleroderma. In embodiments, the Th2/ITK-mediated disease is pulmonary fibrosis. In embodiments, pulmonary fibrosis is idiopathic pulmonary fibrosis. In embodiments, the Th2/ITK-mediated disease is scleroderma. In embodiments, the Th2/ITK-mediated disease is idiopathic pulmonary fibrosis. In embodiments, the Th2/ITK-mediated disease is cirrhosis. In embodiments, the Th2/ITK-mediated disease is retroperitoneal fibrosis. In embodiments, the Th2/ITK-mediated disease is psoriatic arthritis. In embodiments, the Th2/ITK-mediated disease is vasculitis. In embodiments, the Th2/ITK-mediated disease is autoimmune lymphoproliferative syndrome. In embodiments, the Th2/ITK-mediated disease is chronic obstructive pulmonary disease. In embodiments, the Th2/ITK-mediated disease is an eosinophilic disease. In embodiments, the eosinophilic disease is esophagitis. In embodiments, the Th2/ITK-mediated disease is esophagitis. In embodiments, the Th2/ITK-mediated disease is a mast cell disease. In embodiments, the mast cell disease is mastocytosis, mast cell activation syndrome, or hereditary alpha tryptasemia. In embodiments, the Th2/ITK-mediated disease is mastocytosis, mast cell activation syndrome, or hereditary alpha tryptasemia. In embodiments, the Th2/ITK-mediated disease is mastocytosis. In embodiments, the Th2/ITK-mediated disease is mast cell activation syndrome. In embodiments, the Th2/ITK-mediated disease is hereditary alpha tryptasemia. In embodiments, the Th2/ITK-mediated disease is human immunodeficiency viral disease.

The term “Th2/ITK-mediated disease” refers to a disease in which there is an increased expression of ITK and/or an increased Th2 cell response, resulting in the secretion or increased secretion of pro-inflammatory cytokines.

The disclosure provides methods of treating an autoimmune disease or an allergy in a subject in need thereof by measuring a decreased level of Th1⁺ T cells, a decreased ratio of Th1⁺ T cells to Th2⁺ T cells; a decreased ratio of IFNγ⁺CD4⁺ T cells to IL-4⁺CD4⁺ T cells; a decreased level of IFNγ; a decreased level CD8+ cytotoxic lymphocytes; an increased level of Th2+ cells; an increased level of IL-4; an increased level of IL-5; an increased level of IL-10; an increased level of IL-13; an increased level of IL-17; an increased level of Th17+ T cells; an increased level of eosinophils; a decreased level of IL-10, a decreased level of IL-2, a decreased level of IL-12, a decreased level of TNF-α, a decreased level of TNF-γ, a decreased level of GMCS, or a combination of two or more thereof, relative to a control, in a biological sample obtained from the subject, and administering to the subject an effective amount of an ITK inhibitor or a pharmaceutically acceptable salt thereof. The disclosure provides methods of treating an autoimmune disease in a subject in need thereof by measuring an increased level of Th2+ cells; an increased level of IL-4; an increased level of IL-5; an increased level of IL-10; an increased level of IL-13; an increased level of IL-17; or a combination of two or more thereof, relative to a control, in a biological sample obtained from the subject, and administering to the subject an effective amount of an ITK inhibitor or a pharmaceutically acceptable salt thereof. The disclosure provides methods of treating an autoimmune disease in a subject in need thereof by measuring an increased level of IL-4; an increased level of IL-5; an increased level of IL-10; an increased level of IL-13; an increased level of IL-17; or a combination of two or more thereof, relative to a control, in a biological sample obtained from the subject, and administering to the subject an effective amount of an ITK inhibitor or a pharmaceutically acceptable salt thereof. The disclosure provides methods of treating an autoimmune disease in a subject in need thereof by measuring an increased level of IL-4, relative to a control, in a biological sample obtained from the subject, and administering to the subject an effective amount of an ITK inhibitor or a pharmaceutically acceptable salt thereof. The disclosure provides methods of treating an autoimmune disease in a subject in need thereof by measuring an increased level of IL-5, relative to a control, in a biological sample obtained from the subject, and administering to the subject an effective amount of an ITK inhibitor or a pharmaceutically acceptable salt thereof. The disclosure provides methods of treating an autoimmune disease in a subject in need thereof by measuring an increased level of IL-10, relative to a control, in a biological sample obtained from the subject, and administering to the subject an effective amount of an ITK inhibitor or a pharmaceutically acceptable salt thereof. The disclosure provides methods of treating an autoimmune disease in a subject in need thereof by measuring an increased level of IL-13, relative to a control, in a biological sample obtained from the subject, and administering to the subject an effective amount of an ITK inhibitor or a pharmaceutically acceptable salt thereof. The disclosure provides methods of treating an autoimmune disease in a subject in need thereof by measuring an increased level of IL-17, relative to a control, in a biological sample obtained from the subject, and administering to the subject an effective amount of an ITK inhibitor or a pharmaceutically acceptable salt thereof. In embodiments, the method is for treating an autoimmune disease. In embodiments, the method is for treating an allergy. In embodiments, the ITK inhibitor is a compound of Formula (I) or a pharmaceutically acceptable salt thereof. In embodiments, the ITK inhibitor is a compound of Formula (II) or a pharmaceutically acceptable salt thereof. In embodiments, the ITK inhibitor is a compound of Formula (A) or a pharmaceutically acceptable salt thereof. In embodiments, the ITK inhibitor is the free base form of the compound of Formula (A). In embodiments, the ITK inhibitor is a compound of any one of Formula (1) to Formula (34) or a pharmaceutically acceptable salt thereof.

The disclosure provides methods of treating atopic dermatitis, asthma, rhinitis, conjunctivitis, psoriasis, scleroderma, pulmonary fibrosis, cirrhosis, retroperitoneal fibrosis, psoriatic arthritis, vasculitis, autoimmune lymphoproliferative syndrome, chronic obstructive pulmonary disease, esophagitis, mastocytosis, mast cell activation syndrome, hereditary alpha tryptasemia, or human immunodeficiency viral disease in a subject in need thereof by measuring a decreased level of Th1 T cells, a decreased ratio of Th1⁺ T cells to Th2⁺ T cells; a decreased ratio of IFNγ⁺CD4⁺ T cells to IL-4⁺CD4⁺ T cells; a decreased level of IFNγ; a decreased level CD8+ cytotoxic lymphocytes; an increased level of Th2+ cells; an increased level of IL-4; an increased level of IL-5; an increased level of IL-10; an increased level of IL-13; an increased level of IL-17; an increased level of Th17+ T cells; an increased level of eosinophils; a decreased level of IL-1β, a decreased level of IL-2, a decreased level of IL-12, a decreased level of TNF-α, a decreased level of TNF-γ, a decreased level of GMCS, or a combination of two or more thereof, relative to a control, in a biological sample obtained from the subject, and administering to the subject an effective amount of an ITK inhibitor or a pharmaceutically acceptable salt thereof. The disclosure provides methods of treating atopic dermatitis, asthma, rhinitis, conjunctivitis, psoriasis, scleroderma, pulmonary fibrosis, cirrhosis, retroperitoneal fibrosis, psoriatic arthritis, vasculitis, autoimmune lymphoproliferative syndrome, chronic obstructive pulmonary disease, esophagitis, mastocytosis, mast cell activation syndrome, hereditary alpha tryptasemia, or human immunodeficiency viral disease in a subject in need thereof by measuring an increased level of Th2+ cells; an increased level of IL-4; an increased level of IL-5; an increased level of IL-10; an increased level of IL-13; an increased level of IL-17; or a combination of two or more thereof, relative to a control, in a biological sample obtained from the subject, and administering to the subject an effective amount of an ITK inhibitor or a pharmaceutically acceptable salt thereof. The disclosure provides methods of treating atopic dermatitis, asthma, rhinitis, conjunctivitis, psoriasis, scleroderma, pulmonary fibrosis, cirrhosis, retroperitoneal fibrosis, psoriatic arthritis, vasculitis, autoimmune lymphoproliferative syndrome, chronic obstructive pulmonary disease, esophagitis, mastocytosis, mast cell activation syndrome, hereditary alpha tryptasemia, or human immunodeficiency viral disease in a subject in need thereof by measuring an increased level of IL-4; an increased level of IL-5; an increased level of IL-10; an increased level of IL-13; an increased level of IL-17; or a combination of two or more thereof, relative to a control, in a biological sample obtained from the subject, and administering to the subject an effective amount of an ITK inhibitor or a pharmaceutically acceptable salt thereof. In embodiments, the method is for treating atopic dermatitis. In embodiments, the method is for treating asthma. In embodiments, the method is for treating rhinitis. In embodiments, the method is for treating conjunctivitis. In embodiments, the method is for treating psoriasis. In embodiments, the method is for treating scleroderma. In embodiments, the method is for treating pulmonary fibrosis. In embodiments, pulmonary fibrosis is idiopathic pulmonary fibrosis. In embodiments, the method is for treating cirrhosis. In embodiments, the method is for treating retroperitoneal fibrosis. In embodiments, the method is for treating psoriatic arthritis. In embodiments, the method is for treating vasculitis. In embodiments, the method is for treating autoimmune lymphoproliferative syndrome. In embodiments, the method is for treating chronic obstructive pulmonary disease. In embodiments, the method is for treating esophagitis. In embodiments, the method is for treating mastocytosis. In embodiments, the method is for treating mast cell activation syndrome. In embodiments, the method is for treating hereditary alpha tryptasemia. In embodiments, the method is for treating human immunodeficiency viral disease. In embodiments, the ITK inhibitor is a compound of Formula (I) or a pharmaceutically acceptable salt thereof. In embodiments, the ITK inhibitor is a compound of Formula (II) or a pharmaceutically acceptable salt thereof. In embodiments, the ITK inhibitor is a compound of Formula (A) or a pharmaceutically acceptable salt thereof. In embodiments, the ITK inhibitor is the free base form of the compound of Formula (A). In embodiments, the ITK inhibitor is a compound of any one of Formula (1) to Formula (34) or a pharmaceutically acceptable salt thereof.

### Dose and Dosing Regimens

The dosage and frequency (single or multiple doses) of the ITK inhibitors administered to a subject can vary depending upon a variety of factors, for example, whether the mammal suffers from another disease, and its route of administration; size, age, sex, health, body weight, body mass index, and diet of the recipient; nature and extent of symptoms of the disease being treated (e.g. cancer, an autoimmune disease, or an allergy), kind of concurrent treatment, complications from the disease being treated or other health-related problems. Other therapeutic regimens or agents can be used in conjunction with the methods and ITK inhibitors described herein. Adjustment and manipulation of established dosages (e.g., frequency and duration) are well within the ability of those skilled in the art.

For any composition and ITK inhibitor described herein, the effective amount can be initially determined from cell culture assays. Target concentrations will be those concentrations of ITK inhibitors that are capable of achieving the methods described herein, as measured using the methods described herein or known in the art. As is known in the art, effective amounts of ITK inhibitors for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

An “effective amount” is an amount of the ITK inhibitor sufficient to accomplish a stated purpose relative to the absence of the compound (e.g., achieve the effect for which it is administered, such as increase Th1 cell activity, treat cancer, treat an autoimmune disease, treat an allergy, increase Th1 signaling pathway activity, or reduce one or more symptoms of cancer, an autoimmune disease, or an allergy). An example of an “effective amount” of the IKT inhibitor is an amount sufficient to contribute to the treatment, prevention, or reduction of a symptom or symptoms of a disease (e.g., increase Th1 cell activity, treat cancer, treat an autoimmune disease, treat an allergy) which could also be referred to as a “therapeutically effective amount.” A “reduction” of a symptom or symptoms means decreasing of the severity or frequency of the symptoms, or elimination of the symptoms. A “prophylactically effective amount” of an ITK inhibitor is an amount of the ITK inhibitor that, when administered to a subject, will have the intended prophylactic effect, e.g., preventing or delaying the onset (or reoccurrence) of a disease, pathology or condition (e.g., increase Th1 cell activity, treat cancer, treat an autoimmune disease, treat an allergy), or reducing the likelihood of the onset (or reoccurrence) of a disease, pathology, or condition, or their symptoms (e.g., increase Th1 cell activity, treat cancer, treat an autoimmune disease, treat an allergy). The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

Dosages of the ITK inhibitors may be varied depending upon the requirements of the patient. The dose administered to a patient should be sufficient to affect a beneficial therapeutic response in the patient over time. The size of the dose also will be determined by the existence, nature, and extent of any adverse side-effects. Determination of the proper dosage for a particular situation is within the skill of the art. Generally, treatment is initiated with smaller dosages which are less than the optimum dose of the ITK inhibitor. Thereafter, the dosage is increased by small increments until the optimum effect under circumstances is reached. Dosage amounts and intervals can be adjusted individually to provide levels of the ITK inhibitors effective for the particular clinical indication being treated. This will provide a therapeutic regimen that is commensurate with the severity of the individual's disease state.

In embodiments of the methods described herein (e.g., increasing Th1 activity, treating cancer, treating an autoimmune disease, treating an allergy, treating a Th2/ITK-mediated disease, reversing T cell exhaustion), the effective amount of the ITK inhibitor is about 0.5 mmole to about 2.0 mmole of the ITK inhibitor per day. In embodiments, the effective amount is about 0.55 mmole to about 2.0 mmole of the ITK inhibitor per day. In embodiments, the effective amount is about 0.5 mmole to about 1.9 mmole of the ITK inhibitor per day. In embodiments, the effective amount is about 0.6 mmole to about 1.8 mmole of the ITK inhibitor per day. In embodiments, the effective amount is about 0.6 mmole to about 1.7 mmole of the ITK inhibitor per day. In embodiments, the effective amount is about 0.6 mmole to about 1.6 mmole of the ITK inhibitor per day. In embodiments, the effective amount is about 0.6 mmole to about 1.55 mmole of the ITK inhibitor per day. In embodiments, the effective amount is about 0.6 mmole to about 1.5 mmole of the ITK inhibitor per day. In embodiments, the effective amount is about 0.6 mmole to about 1.45 mmole of the ITK inhibitor per day. In embodiments, the effective amount is about 0.6 mmole to about 1.4 mmole of the ITK inhibitor per day. In embodiments, the effective amount is about 0.6 mmole to about 1.35 mmole of the ITK inhibitor per day. In embodiments, the effective amount is about 0.6 mmole to about 1.3 mmole of the ITK inhibitor per day. In embodiments, the effective amount is about 0.6 mmole to about 1.25 mmole of the ITK inhibitor per day. In embodiments, the effective amount is about 0.6 mmole to about 1.2 mmole of the ITK inhibitor per day. In embodiments, the effective amount is about 0.6 mmole to about 1.15 mmole of the ITK inhibitor per day. In embodiments, the effective amount is about 0.6 mmole to about 1.1 mmole of the ITK inhibitor per day. In embodiments, the effective amount is about 0.6 mmole to about 1.05 mmole of the ITK inhibitor per day.

In embodiments of the methods described herein (e.g., increasing Th1 activity, treating cancer, treating an autoimmune disease, treating an allergy, treating a Th2/ITK-mediated disease, reversing T cell exhaustion), the effective amount is about 0.6 mmole to about 1.0 mmole of the ITK inhibitor per day. In embodiments, the effective amount is about 0.61 mmole to about 0.99 mmole of the ITK inhibitor per day. In embodiments, the effective amount is about 0.62 mmole to about 0.98 mmole of the ITK inhibitor per day. In embodiments, the effective amount is about 0.63 mmole to about 0.97 mmole of the ITK inhibitor per day. In embodiments, the effective amount is about 0.64 mmole to about 0.96 mmole of the ITK inhibitor per day. In embodiments, the effective amount is about 0.65 mmole to about 0.95 mmole of the ITK inhibitor per day. In embodiments, the effective amount is about 0.66 mmole to about 0.94 mmole of the ITK inhibitor per day. In embodiments, the effective amount is about 0.67 mmole to about 0.93 mmole of the ITK inhibitor per day. In embodiments, the effective amount is about 0.68 mmole to about 0.92 mmole of the ITK inhibitor per day. In embodiments, the effective amount is about 0.69 mmole to about 0.91 mmole of the ITK inhibitor per day.

In embodiments of the methods described herein (e.g., increasing Th1 activity, treating cancer, treating an autoimmune disease, treating an allergy, treating a Th2/ITK-mediated disease, reversing T cell exhaustion), the effective amount is about 0.7 mmole to about 0.9 mmole of the ITK inhibitor per day. In embodiments, the effective amount is about 0.71 mmole to about 0.89 mmole of the ITK inhibitor per day. In embodiments, the effective amount is about 0.72 mmole to about 0.88 mmole of the ITK inhibitor per day. In embodiments, the effective amount is about 0.73 mmole to about 0.87 mmole of the ITK inhibitor per day. In embodiments, the effective amount is about 0.74 mmole to about 0.86 mmole of the ITK inhibitor per day. In embodiments, the effective amount is about 0.75 mmole to about 0.85 mmole of the ITK inhibitor per day. In embodiments, the effective amount is about 0.76 mmole to about 0.84 mmole of the ITK inhibitor per day. In embodiments, the effective amount is about 0.77 mmole to about 0.83 mmole of the ITK inhibitor per day. In embodiments, the effective amount is about 0.78 mmole to about 0.82 mmole of the ITK inhibitor per day. In embodiments, the effective amount is about 0.79 mmole to about 0.81 mmole of the ITK inhibitor per day. In embodiments, the effective amount is about 0.8 mmole of the ITK inhibitor per day.

In embodiments of the methods described herein (e.g., increasing Th1 activity, treating cancer, treating an autoimmune disease, treating an allergy, treating a Th2/ITK-mediated disease, reversing T cell exhaustion), the effective amount is about 0.25 mmole to about 1.0 mmole of the ITK inhibitor twice per day (BID). In embodiments, the effective amount is about 0.275 mmole to about 1.0 mmole of the ITK inhibitor twice per day. In embodiments, the effective amount is about 0.25 mmole to about 0.95 mmole of the ITK inhibitor twice per day. In embodiments, the effective amount is about 0.3 mmole to about 0.9 mmole of the ITK inhibitor twice per day. In embodiments, the effective amount is about 0.3 mmole to about 0.85 mmole of the ITK inhibitor twice per day. In embodiments, the effective amount is about 0.3 mmole to about 0.8 mmole of the ITK inhibitor twice per day. In embodiments, the effective amount is about 0.3 mmole to about 0.775 mmole of the ITK inhibitor twice per day. In embodiments, the effective amount is about 0.3 mmole to about 0.75 mmole of the ITK inhibitor twice per day. In embodiments, the effective amount is about 0.3 mmole to about 0.725 mmole of the ITK inhibitor twice per day. In embodiments, the effective amount is about 0.3 mmole to about 0.7 mmole of the ITK inhibitor twice per day. In embodiments, the effective amount is about 0.3 mmole to about 0.675 mmole of the ITK inhibitor twice per day. In embodiments, the effective amount is about 0.3 mmole to about 0.65 mmole of the ITK inhibitor twice per day. In embodiments, the effective amount is about 0.3 mmole to about 0.625 mmole of the ITK inhibitor twice per day. In embodiments, the effective amount is about 0.3 mmole to about 0.6 mmole of the ITK inhibitor twice per day. In embodiments, the effective amount is about 0.3 mmole to about 0.575 mmole of the ITK inhibitor twice per day. In embodiments, the effective amount is about 0.3 mmole to about 0.55 mmole of the ITK inhibitor twice per day. In embodiments, the effective amount is about 0.3 mmole to about 0.525 mmole of the ITK inhibitor twice per day.

In embodiments of the methods described herein (e.g., increasing Th1 activity, treating cancer, treating an autoimmune disease, treating an allergy, treating a Th2/ITK-mediated disease, reversing T cell exhaustion), the effective amount is about 0.3 mmole to about 0.5 mmole of the ITK inhibitor twice per day. In embodiments, the effective amount is about 0.305 mmole to about 0.495 mmole of the ITK inhibitor twice per day. In embodiments, the effective amount is about 0.31 mmole to about 0.49 mmole of the ITK inhibitor twice per day. In embodiments, the effective amount is about 0.315 mmole to about 0.485 mmole of the ITK inhibitor twice per day. In embodiments, the effective amount is about 0.32 mmole to about 0.48 mmole of the ITK inhibitor twice per day. In embodiments, the effective amount is about 0.325 mmole to about 0.475 mmole of the ITK inhibitor twice per day. In embodiments, the effective amount is about 0.33 mmole to about 0.47 mmole of the ITK inhibitor twice per day. In embodiments, the effective amount is about 0.335 mmole to about 0.465 mmole of the ITK inhibitor twice per day. In embodiments, the effective amount is about 0.34 mmole to about 0.46 mmole of the ITK inhibitor twice per day. In embodiments, the effective amount is about 0.345 mmole to about 0.455 mmole of the ITK inhibitor twice per day.

In embodiments of the methods described herein (e.g., increasing Th1 activity, treating cancer, treating an autoimmune disease, treating an allergy, treating a Th2/ITK-mediated disease, reversing T cell exhaustion), the effective amount is about 0.35 mmole to about 0.45 mmole of the ITK inhibitor twice per day. In embodiments, the effective amount is about 0.355 mmole to about 0.445 mmole of the ITK inhibitor twice per day. In embodiments, the effective amount is about 0.36 mmole to about 0.44 mmole of the ITK inhibitor twice per day. In embodiments, the effective amount is about 0.365 mmole to about 0.435 mmole of the ITK inhibitor twice per day. In embodiments, the effective amount is about 0.37 mmole to about 0.43 mmole of the ITK inhibitor twice per day. In embodiments, the effective amount is about 0.375 mmole to about 0.425 mmole of the ITK inhibitor twice per day. In embodiments, the effective amount is about 0.38 mmole to about 0.42 mmole of the ITK inhibitor twice per day. In embodiments, the effective amount is about 0.385 mmole to about 0.415 mmole of the ITK inhibitor twice per day. In embodiments, the effective amount is about 0.39 mmole to about 0.41 mmole of the ITK inhibitor twice per day. In embodiments, the effective amount is about 0.395 mmole to about 0.405 mmole of the ITK inhibitor twice per day. In embodiments, the effective amount is about 0.4 mmole of the ITK inhibitor twice per day.

In embodiments of the methods described herein (e.g., increasing Th1 activity, treating cancer, treating an autoimmune disease, treating an allergy, treating a Th2/ITK-mediated disease, reversing T cell exhaustion), the effective amount is about 250 mg to about 1,000 mg of the ITK inhibitor per day. In embodiments, the effective amount is about 250 mg to about 950 mg of the ITK inhibitor per day. In embodiments, the effective amount is about 250 mg to about 900 mg of the ITK inhibitor per day. In embodiments, the effective amount is about 250 mg to about 850 mg of the ITK inhibitor per day. In embodiments, the effective amount is about 250 mg to about 800 mg of the ITK inhibitor per day. In embodiments, the effective amount is about 250 mg to about 750 mg of the ITK inhibitor per day. In embodiments, the effective amount is about 250 mg to about 700 mg of the ITK inhibitor per day. In embodiments, the effective amount is about 250 mg to about 650 mg of the ITK inhibitor per day. In embodiments, the effective amount is about 250 mg to about 600 mg of the ITK inhibitor per day. In embodiments, the effective amount is about 250 mg to about 550 mg of the ITK inhibitor per day.

In embodiments of the methods described herein (e.g., increasing Th1 activity, treating cancer, treating an autoimmune disease, treating an allergy, treating a Th2/ITK-mediated disease, reversing T cell exhaustion), the effective amount is about 300 mg to about 1,000 mg of the ITK inhibitor per day. In embodiments, the effective amount is about 300 mg to about 950 mg of the ITK inhibitor per day. In embodiments, the effective amount is about 300 mg to about 900 mg of the ITK inhibitor per day. In embodiments, the effective amount is about 300 mg to about 850 mg of the ITK inhibitor per day. In embodiments, the effective amount is about 300 mg to about 800 mg of the ITK inhibitor per day. In embodiments, the effective amount is about 300 mg to about 750 mg of the ITK inhibitor per day. In embodiments, the effective amount is about 300 mg to about 700 mg of the ITK inhibitor per day. In embodiments, the effective amount is about 300 mg to about 650 mg of the ITK inhibitor per day. In embodiments, the effective amount is about 300 mg to about 600 mg of the ITK inhibitor per day. In embodiments, the effective amount is about 300 mg to about 550 mg of the ITK inhibitor per day.

In embodiments of the methods described herein (e.g., increasing Th1 activity, treating cancer, treating an autoimmune disease, treating an allergy, treating a Th2/ITK-mediated disease, reversing T cell exhaustion), the effective amount is about 300 mg to about 500 mg of the ITK inhibitor per day. In embodiments, the effective amount is about 305 mg to about 495 mg of the ITK inhibitor per day. In embodiments, the effective amount is about 310 mg to about 490 mg of the ITK inhibitor per day. In embodiments, the effective amount is about 315 mg to about 485 mg of the ITK inhibitor per day. In embodiments, the effective amount is about 320 mg to about 480 mg of the ITK inhibitor per day. In embodiments, the effective amount is about 325 mg to about 475 mg of the ITK inhibitor per day. In embodiments, the effective amount is about 330 mg to about 470 mg of the ITK inhibitor per day. In embodiments, the effective amount is about 335 mg to about 465 mg of the ITK inhibitor per day. In embodiments, the effective amount is about 340 mg to about 460 mg of the ITK inhibitor per day. In embodiments, the effective amount is about 345 mg to about 455 mg of the ITK inhibitor per day. In embodiments, the effective amount is about 350 mg to about 450 mg of the ITK inhibitor per day. In embodiments, the effective amount is about 355 mg to about 445 mg of the ITK inhibitor per day. In embodiments, the effective amount is about 360 mg to about 440 mg of the ITK inhibitor per day. In embodiments, the effective amount is about 365 mg to about 435 mg of the ITK inhibitor per day. In embodiments, the effective amount is about 370 mg to about 430 mg of the ITK inhibitor per day. In embodiments, the effective amount is about 375 mg to about 425 mg of the ITK inhibitor per day. In embodiments, the effective amount is about 380 mg to about 420 mg of the ITK inhibitor per day. In embodiments, the effective amount is about 385 mg to about 415 mg of the ITK inhibitor per day. In embodiments, the effective amount is about 390 mg to about 410 mg of the ITK inhibitor per day. In embodiments, the effective amount is about 395 mg to about 405 mg of the ITK inhibitor per day. In embodiments, the effective amount is about 400 mg of the ITK inhibitor per day.

In embodiments of the methods described herein (e.g., increasing Th1 activity, treating cancer, treating an autoimmune disease, treating an allergy, treating a Th2/ITK-mediated disease, reversing T cell exhaustion), the effective amount is about 125 mg to about 500 mg of the ITK inhibitor twice per day (BID). In embodiments, the effective amount is about 125 mg to about 475 mg of the ITK inhibitor twice per day. In embodiments, the effective amount is about 125 mg to about 450 mg of the ITK inhibitor twice per day. In embodiments, the effective amount is about 125 mg to about 425 mg of the ITK inhibitor twice per day. In embodiments, the effective amount is about 125 mg to about 400 mg of the ITK inhibitor twice per day. In embodiments, the effective amount is about 125 mg to about 375 mg of the ITK inhibitor twice per day. In embodiments, the effective amount is about 125 mg to about 350 mg of the ITK inhibitor twice per day. In embodiments, the effective amount is about 125 mg to about 325 mg of the ITK inhibitor twice per day. In embodiments, the effective amount is about 125 mg to about 300 mg of the ITK inhibitor twice per day. In embodiments, the effective amount is about 125 mg to about 275 mg of the ITK inhibitor twice per day.

In embodiments of the methods described herein (e.g., increasing Th1 activity, treating cancer, treating an autoimmune disease, treating an allergy, treating a Th2/ITK-mediated disease, reversing T cell exhaustion), the effective amount is about 150 mg to about 500 mg of the ITK inhibitor twice per day. In embodiments, the effective amount is about 150 mg to about 475 mg of the ITK inhibitor twice per day. In embodiments, the effective amount is about 150 mg to about 450 mg of the ITK inhibitor twice per day. In embodiments, the effective amount is about 150 mg to about 425 mg of the ITK inhibitor twice per day. In embodiments, the effective amount is about 150 mg to about 400 mg of the ITK inhibitor twice per day. In embodiments, the effective amount is about 150 mg to about 375 mg of the ITK inhibitor twice per day. In embodiments, the effective amount is about 150 mg to about 350 mg of the ITK inhibitor twice per day. In embodiments, the effective amount is about 150 mg to about 325 mg of the ITK inhibitor twice per day. In embodiments, the effective amount is about 150 mg to about 300 mg of the ITK inhibitor twice per day. In embodiments, the effective amount is about 150 mg to about 275 mg of the ITK inhibitor twice per day.

In embodiments of the methods described herein (e.g., increasing Th1 activity, treating cancer, treating an autoimmune disease, treating an allergy, treating a Th2/ITK-mediated disease, reversing T cell exhaustion), the effective amount is about 150 mg to about 250 mg of the ITK inhibitor twice per day. In embodiments, the effective amount is about 152.5 mg to about 247.5 mg of the ITK inhibitor twice per day. In embodiments, the effective amount is about 155 mg to about 245 mg of the ITK inhibitor twice per day. In embodiments, the effective amount is about 157.5 mg to about 242.5 mg of the ITK inhibitor twice per day. In embodiments, the effective amount is about 160 mg to about 240 mg of the ITK inhibitor twice per day. In embodiments, the effective amount is about 162.5 mg to about 237.5 mg of the ITK inhibitor twice per day. In embodiments, the effective amount is about 165 mg to about 235 mg of the ITK inhibitor twice per day. In embodiments, the effective amount is about 167.5 mg to about 232.5 mg of the ITK inhibitor twice per day. In embodiments, the effective amount is about 170 mg to about 230 mg of the ITK inhibitor twice per day. In embodiments, the effective amount is about 172.5 mg to about 227.5 mg of the ITK inhibitor twice per day. In embodiments, the effective amount is about 175 mg to about 225 mg of the ITK inhibitor twice per day. In embodiments, the effective amount is about 177.5 mg to about 222.5 mg of the ITK inhibitor twice per day. In embodiments, the effective amount is about 180 mg to about 220 mg of the ITK inhibitor twice per day. In embodiments, the effective amount is about 182.5 mg to about 217.5 mg of the ITK inhibitor twice per day. In embodiments, the effective amount is about 185 mg to about 215 mg of the ITK inhibitor twice per day. In embodiments, the effective amount is about 187.5 mg to about 212.5 mg of the ITK inhibitor twice per day. In embodiments, the effective amount is about 190 mg to about 210 mg of the ITK inhibitor twice per day. In embodiments, the effective amount is about 192.5 mg to about 207.5 mg of the ITK inhibitor twice per day. In embodiments, the effective amount is about 195 mg to about 205 mg of the ITK inhibitor twice per day. In embodiments, the effective amount is about 197.5 mg to about 202.5 mg of the ITK inhibitor twice per day. In embodiments, the effective amount is about 200 mg of the ITK inhibitor twice per day.

In embodiments, the methods described herein (e.g., increasing Th1 activity, treating cancer, treating an autoimmune disease, treating an allergy, treating a Th2/ITK-mediated disease, reversing T cell exhaustion) comprise a drug holiday. A “drug holiday” is a period of time, anywhere from a few days to a few months, when a patient discontinues taking medication for their treatment. A drug holiday can have a therapeutic benefit, e.g., allowing a drug to regain therapeutic benefit after a period of continuous use. In embodiments, the disclosure provides a method comprising the steps of (in order): (1) administering the ITK inhibitor for about 2 weeks to about 12 weeks; (2) discontinuing administration of the ITK inhibitor for about I week to about 8 weeks (i.e., drug holiday), and (3) administering the ITK inhibitor for at least 2 weeks. In embodiments, the disclosure provides a method comprising the steps of (in order): (1) administering the ITK inhibitor for about 4 weeks to about 12 weeks; (2) discontinuing administration of the ITK inhibitor for about 1 week to about 8 weeks (i.e., drug holiday), and (3) administering the ITK inhibitor for at least 2 weeks. In embodiments, the disclosure provides a method comprising the steps of (in order): (1) administering the ITK inhibitor for about 3 weeks to about 12 weeks; (2) discontinuing administration of the ITK inhibitor for about 1 week to about 8 weeks (i.e., drug holiday), and (3) administering the ITK inhibitor for at least 2 weeks. In embodiments, the method comprises the steps of (in order): (1) administering the ITK inhibitor for about 4 weeks to about 12 weeks; (2) discontinuing administration of the ITK inhibitor for about 1 week to about 8 weeks (i.e., drug holiday), and (3) administering the ITK inhibitor for at least 4 weeks.

In embodiments, the method (e.g., increasing Th1 activity, treating cancer, treating an autoimmune disease, treating an allergy, treating a Th2/ITK-mediated disease, reversing T cell exhaustion) comprises the steps of (in order): (1) administering the ITK inhibitor for about 4 weeks to about 12 weeks; (2) discontinuing administration of the ITK inhibitor for about 1 weeks to about 6 weeks, and (3) administering the ITK inhibitor for at least 4 weeks. In embodiments, the method comprises the steps of (in order): (1) administering the ITK inhibitor for about 4 weeks to about 12 weeks; (2) discontinuing administration of the ITK inhibitor for about 1 weeks to about 5 weeks, and (3) administering the ITK inhibitor for at least 4 weeks. In embodiments, the method comprises the steps of (in order): (1) administering the ITK inhibitor for about 2 weeks to about 12 weeks; (2) discontinuing administration of the ITK inhibitor for about 1 week to about 4 weeks, and (3) administering the ITK inhibitor for at least 4 weeks. In embodiments, the method comprises the steps of (in order): (1) administering the ITK inhibitor for about 4 weeks to about 12 weeks; (2) discontinuing administration of the ITK inhibitor for about 1 weeks to about 3 weeks, and (3) administering the ITK inhibitor for at least 4 weeks. In embodiments, the method comprises the steps of (in order): (1) administering the ITK inhibitor for about 4 weeks to about 12 weeks; (2) discontinuing administration of the ITK inhibitor for about 1 weeks to about 2 weeks, and (3) administering the ITK inhibitor for at least 4 weeks.

In embodiments, the method (e.g., increasing Th1 activity, treating cancer, treating an autoimmune disease, treating an allergy, treating a Th2/ITK-mediated disease, reversing T cell exhaustion) comprises the steps of (in order): (1) administering the ITK inhibitor for about 2 weeks to about 12 weeks; (2) discontinuing administration of the ITK inhibitor for about 2 weeks to about 4 weeks, and (3) administering the ITK inhibitor for at least 4 weeks. In embodiments, the method comprises the steps of (in order): (1) administering the ITK inhibitor for about 2 weeks to about 11 weeks; (2) discontinuing administration of the ITK inhibitor for about 2 weeks to about 4 weeks, and (3) administering the ITK inhibitor for at least 4 weeks. In embodiments, the method comprises the steps of (in order): (1) administering the ITK inhibitor for about 2 weeks to about 10 weeks; (2) discontinuing administration of the ITK inhibitor for about 2 weeks to about 4 weeks, and (3) administering the ITK inhibitor for at least 4 weeks. In embodiments, the method comprises the steps of (in order): (1) administering the ITK inhibitor for about 2 weeks to about 9 weeks; (2) discontinuing administration of the ITK inhibitor for about 2 weeks to about 4 weeks, and (3) administering the ITK inhibitor for at least 4 weeks. In embodiments, the method comprises the steps of (in order): (1) administering the ITK inhibitor for about 2 weeks to about 8 weeks; (2) discontinuing administration of the ITK inhibitor for about 2 weeks to about 4 weeks, and (3) administering the ITK inhibitor for at least 4 weeks. In embodiments, the method comprises the steps of (in order): (1) administering the ITK inhibitor for about 2 weeks to about 7 weeks; (2) discontinuing administration of the ITK inhibitor for about 2 weeks to about 4 weeks, and (3) administering the ITK inhibitor for at least 4 weeks. In embodiments, the method comprises the steps of (in order): (1) administering the ITK inhibitor for about 2 weeks to about 6 weeks; (2) discontinuing administration of the ITK inhibitor for about 2 weeks to about 4 weeks, and (3) administering the ITK inhibitor for at least 4 weeks. In embodiments, the method comprises the steps of (in order): (1) administering the ITK inhibitor for about 2 weeks to about 5 weeks; (2) discontinuing administration of the ITK inhibitor for about 2 weeks to about 4 weeks, and (3) administering the ITK inhibitor for at least 4 weeks. In embodiments, the method comprises the steps of (in order): (1) administering the ITK inhibitor for about 2 weeks to about 4 weeks; (2) discontinuing administration of the ITK inhibitor for about 2 weeks to about 4 weeks, and (3) administering the ITK inhibitor for at least 4 weeks.

In embodiments, the method (e.g., increasing Th1 activity, treating cancer, treating an autoimmune disease, treating an allergy, treating a Th2/ITK-mediated disease, reversing T cell exhaustion) comprises the steps of (in order): (1) administering the ITK inhibitor for about 2 weeks to about 12 weeks; (2) discontinuing administration of the ITK inhibitor for about 2 weeks to about 6 weeks, and (3) administering the ITK inhibitor for at least 4 weeks. In embodiments, the method comprises the steps of (in order): (1) administering the ITK inhibitor for about 2 weeks to about 11 weeks; (2) discontinuing administration of the ITK inhibitor for about 2 weeks to about 6 weeks, and (3) administering the ITK inhibitor for at least 4 weeks. In embodiments, the method comprises the steps of (in order): (1) administering the ITK inhibitor for about 2 weeks to about 10 weeks; (2) discontinuing administration of the ITK inhibitor for about 2 weeks to about 4 weeks, and (3) administering the ITK inhibitor for at least 4 weeks. In embodiments, the method comprises the steps of (in order): (1) administering the ITK inhibitor for about 2 weeks to about 9 weeks; (2) discontinuing administration of the ITK inhibitor for about 2 weeks to about 6 weeks, and (3) administering the ITK inhibitor for at least 4 weeks. In embodiments, the method comprises the steps of (in order): (1) administering the ITK inhibitor for about 2 weeks to about 8 weeks; (2) discontinuing administration of the ITK inhibitor for about 2 weeks to about 6 weeks, and (3) administering the ITK inhibitor for at least 4 weeks. In embodiments, the method comprises the steps of (in order): (1) administering the ITK inhibitor for about 2 weeks to about 7 weeks; (2) discontinuing administration of the ITK inhibitor for about 2 weeks to about 4 weeks, and (3) administering the ITK inhibitor for at least 4 weeks. In embodiments, the method comprises the steps of (in order): (1) administering the ITK inhibitor for about 2 weeks to about 6 weeks; (2) discontinuing administration of the ITK inhibitor for about 2 weeks to about 6 weeks, and (3) administering the ITK inhibitor for at least 4 weeks. In embodiments, the method comprises the steps of (in order): (1) administering the ITK inhibitor for about 2 weeks to about 5 weeks; (2) discontinuing administration of the ITK inhibitor for about 2 weeks to about 6 weeks, and (3) administering the ITK inhibitor for at least 4 weeks. In embodiments, the method comprises the steps of (in order): (1) administering the ITK inhibitor for about 2 weeks to about 4 weeks; (2) discontinuing administration of the ITK inhibitor for about 2 weeks to about 6 weeks, and (3) administering the ITK inhibitor for at least 4 weeks.

In embodiments, the method (e.g., increasing Th1 activity, treating cancer, treating an autoimmune disease, treating an allergy, treating a Th2/ITK-mediated disease, reversing T cell exhaustion) comprises the steps of (in order): (1) administering the ITK inhibitor for about 2 weeks to about 12 weeks; (2) discontinuing administration of the ITK inhibitor for about 1 week to about 4 weeks, and (3) administering the ITK inhibitor for at least 4 weeks. In embodiments, the method comprises the steps of (in order): (1) administering the ITK inhibitor for about 2 weeks to about 11 weeks; (2) discontinuing administration of the ITK inhibitor for about 1 week to about 4 weeks, and (3) administering the ITK inhibitor for at least 4 weeks. In embodiments, the method comprises the steps of (in order): (1) administering the ITK inhibitor for about 2 weeks to about 10 weeks; (2) discontinuing administration of the ITK inhibitor for about 1 week to about 4 weeks, and (3) administering the ITK inhibitor for at least 4 weeks. In embodiments, the method comprises the steps of (in order): (1) administering the ITK inhibitor for about 2 weeks to about 9 weeks; (2) discontinuing administration of the ITK inhibitor for about 1 week to about 4 weeks, and (3) administering the ITK inhibitor for at least 4 weeks. In embodiments, the method comprises the steps of (in order): (1) administering the ITK inhibitor for about 2 weeks to about 8 weeks; (2) discontinuing administration of the ITK inhibitor for about 1 week to about 4 weeks, and (3) administering the ITK inhibitor for at least 4 weeks. In embodiments, the method comprises the steps of (in order): (1) administering the ITK inhibitor for about 2 weeks to about 7 weeks; (2) discontinuing administration of the ITK inhibitor for about 1 week to about 4 weeks, and (3) administering the ITK inhibitor for at least 4 weeks. In embodiments, the method comprises the steps of (in order): (1) administering the ITK inhibitor for about 2 weeks to about 6 weeks; (2) discontinuing administration of the ITK inhibitor for about 1 week to about 4 weeks, and (3) administering the ITK inhibitor for at least 4 weeks. In embodiments, the method comprises the steps of (in order): (1) administering the ITK inhibitor for about 2 weeks to about 5 weeks; (2) discontinuing administration of the ITK inhibitor for about 1 week to about 4 weeks, and (3) administering the ITK inhibitor for at least 4 weeks. In embodiments, the method comprises the steps of (in order): (1) administering the ITK inhibitor for about 2 weeks to about 4 weeks; (2) discontinuing administration of the ITK inhibitor for about 1 week to about 4 weeks, and (3) administering the ITK inhibitor for at least 4 weeks.

In embodiments, the method (e.g., increasing Th1 activity, treating cancer, treating an autoimmune disease, treating an allergy, treating a Th2/ITK-mediated disease, reversing T cell exhaustion) comprises the steps of (in order): (1) administering the ITK inhibitor for about 2 weeks to about 12 weeks; (2) discontinuing administration of the ITK inhibitor for about 2 weeks to about 4 weeks, and (3) administering the ITK inhibitor for at least 2 weeks. In embodiments, the method comprises the steps of (in order): (1) administering the ITK inhibitor for about 2 weeks to about 11 weeks; (2) discontinuing administration of the ITK inhibitor for about 2 weeks to about 4 weeks, and (3) administering the ITK inhibitor for at least 2 weeks. In embodiments, the method comprises the steps of (in order): (1) administering the ITK inhibitor for about 2 weeks to about 10 weeks; (2) discontinuing administration of the ITK inhibitor for about 2 weeks to about 4 weeks, and (3) administering the ITK inhibitor for at least 2 weeks. In embodiments, the method comprises the steps of (in order): (1) administering the ITK inhibitor for about 2 weeks to about 9 weeks; (2) discontinuing administration of the ITK inhibitor for about 2 weeks to about 4 weeks, and (3) administering the ITK inhibitor for at least 4 weeks. In embodiments, the method comprises the steps of (in order): (1) administering the ITK inhibitor for about 2 weeks to about 8 weeks; (2) discontinuing administration of the ITK inhibitor for about 2 weeks to about 4 weeks, and (3) administering the ITK inhibitor for at least 2 weeks. In embodiments, the method comprises the steps of (in order): (1) administering the ITK inhibitor for about 2 weeks to about 7 weeks; (2) discontinuing administration of the ITK inhibitor for about 2 weeks to about 4 weeks, and (3) administering the ITK inhibitor for at least 2 weeks. In embodiments, the method comprises the steps of (in order): (1) administering the ITK inhibitor for about 2 weeks to about 6 weeks; (2) discontinuing administration of the ITK inhibitor for about 2 weeks to about 4 weeks, and (3) administering the ITK inhibitor for at least 2 weeks. In embodiments, the method comprises the steps of (in order): (1) administering the ITK inhibitor for about 2 weeks to about 5 weeks; (2) discontinuing administration of the ITK inhibitor for about 2 weeks to about 4 weeks, and (3) administering the ITK inhibitor for at least 2 weeks. In embodiments, the method comprises the steps of (in order): (1) administering the ITK inhibitor for about 2 weeks to about 4 weeks; (2) discontinuing administration of the ITK inhibitor for about 2 weeks to about 4 weeks, and (3) administering the ITK inhibitor for at least 2 weeks.

In embodiments, the method (e.g., increasing Th1 activity, treating cancer, treating an autoimmune disease, treating an allergy, treating a Th2/ITK-mediated disease, reversing T cell exhaustion) comprises the steps of (in order): (1) administering the ITK inhibitor for about 2 weeks to about 12 weeks; (2) discontinuing administration of the ITK inhibitor for about 2 weeks to about 6 weeks, and (3) administering the ITK inhibitor for at least 4 weeks. In embodiments, the method comprises the steps of (in order): (1) administering the ITK inhibitor for about 2 weeks to about 11 weeks; (2) discontinuing administration of the ITK inhibitor for about 2 weeks to about 6 weeks, and (3) administering the ITK inhibitor for at least 2 weeks. In embodiments, the method comprises the steps of (in order): (1) administering the ITK inhibitor for about 2 weeks to about 10 weeks; (2) discontinuing administration of the ITK inhibitor for about 2 weeks to about 4 weeks, and (3) administering the ITK inhibitor for at least 2 weeks. In embodiments, the method comprises the steps of (in order): (1) administering the ITK inhibitor for about 2 weeks to about 9 weeks; (2) discontinuing administration of the ITK inhibitor for about 2 weeks to about 6 weeks, and (3) administering the ITK inhibitor for at least 2 weeks. In embodiments, the method comprises the steps of (in order): (1) administering the ITK inhibitor for about 2 weeks to about 8 weeks; (2) discontinuing administration of the ITK inhibitor for about 2 weeks to about 6 weeks, and (3) administering the ITK inhibitor for at least 2 weeks. In embodiments, the method comprises the steps of (in order): (1) administering the ITK inhibitor for about 2 weeks to about 7 weeks; (2) discontinuing administration of the ITK inhibitor for about 2 weeks to about 4 weeks, and (3) administering the ITK inhibitor for at least 2 weeks. In embodiments, the method comprises the steps of (in order): (1) administering the ITK inhibitor for about 2 weeks to about 6 weeks; (2) discontinuing administration of the ITK inhibitor for about 2 weeks to about 6 weeks, and (3) administering the ITK inhibitor for at least 2 weeks. In embodiments, the method comprises the steps of (in order): (1) administering the ITK inhibitor for about 2 weeks to about 5 weeks; (2) discontinuing administration of the ITK inhibitor for about 2 weeks to about 6 weeks, and (3) administering the ITK inhibitor for at least 2 weeks. In embodiments, the method comprises the steps of (in order): (1) administering the ITK inhibitor for about 2 weeks to about 4 weeks; (2) discontinuing administration of the ITK inhibitor for about 2 weeks to about 6 weeks, and (3) administering the ITK inhibitor for at least 2 weeks.

In embodiments, the method (e.g., increasing Th1 activity, treating cancer, treating an autoimmune disease, treating an allergy, treating a Th2/ITK-mediated disease, reversing T cell exhaustion) comprises the steps of (in order): (1) administering the ITK inhibitor for about 2 weeks to about 12 weeks; (2) discontinuing administration of the ITK inhibitor for about 1 week to about 4 weeks, and (3) administering the ITK inhibitor for at least 2 weeks. In embodiments, the method comprises the steps of (in order): (1) administering the ITK inhibitor for about 2 weeks to about 11 weeks; (2) discontinuing administration of the ITK inhibitor for about 1 week to about 4 weeks, and (3) administering the ITK inhibitor for at least 2 weeks. In embodiments, the method comprises the steps of (in order): (1) administering the ITK inhibitor for about 2 weeks to about 10 weeks; (2) discontinuing administration of the ITK inhibitor for about 1 week to about 4 weeks, and (3) administering the ITK inhibitor for at least 2 weeks. In embodiments, the method comprises the steps of (in order): (1) administering the ITK inhibitor for about 2 weeks to about 9 weeks; (2) discontinuing administration of the ITK inhibitor for about 1 week to about 4 weeks, and (3) administering the ITK inhibitor for at least 2 weeks. In embodiments, the method comprises the steps of (in order): (1) administering the ITK inhibitor for about 2 weeks to about 8 weeks; (2) discontinuing administration of the ITK inhibitor for about 1 week to about 4 weeks, and (3) administering the ITK inhibitor for at least 2 weeks. In embodiments, the method comprises the steps of (in order): (1) administering the ITK inhibitor for about 2 weeks to about 7 weeks; (2) discontinuing administration of the ITK inhibitor for about 1 week to about 4 weeks, and (3) administering the ITK inhibitor for at least 2 weeks. In embodiments, the method comprises the steps of (in order): (1) administering the ITK inhibitor for about 2 weeks to about 6 weeks; (2) discontinuing administration of the ITK inhibitor for about 1 week to about 4 weeks, and (3) administering the ITK inhibitor for at least 4 weeks. In embodiments, the method comprises the steps of (in order): (1) administering the ITK inhibitor for about 2 weeks to about 5 weeks; (2) discontinuing administration of the ITK inhibitor for about 1 week to about 4 weeks, and (3) administering the ITK inhibitor for at least 2 weeks. In embodiments, the method comprises the steps of (in order): (1) administering the ITK inhibitor for about 2 weeks to about 4 weeks; (2) discontinuing administration of the ITK inhibitor for about 1 week to about 4 weeks, and (3) administering the ITK inhibitor for at least 2 weeks.

### Pharmaceutical Compositions

Provided herein are pharmaceutical compositions comprising an ITK inhibitor and a pharmaceutically acceptable excipient. The compositions are suitable for formulation and administration in vitro or in vivo. Suitable carriers and excipients and their formulations are described in Remington: The Science and Practice of Pharmacy, 21st Edition, David B. Troy, ed., Lippicott Williams & Wilkins (2005).

“Pharmaceutically acceptable excipient” and “pharmaceutically acceptable carrier” refer to a substance that aids the administration of an active agent to and absorption by a subject and can be included in the compositions of the disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful.

Solutions of the active compounds as free base or pharmacologically acceptable salt can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5 to 7. In addition, antimicrobial preservatives, similar to those used in ophthalmic preparations and appropriate drug stabilizers, if required, may be included in the formulation. Various commercial nasal preparations are known and can include, for example, antibiotics and antihistamines.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In embodiments, oral pharmaceutical compositions will comprise an inert diluent or edible carrier, or they may be enclosed in hard or soft shell gelatin capsule, or they may be compressed into tablets, or they may be incorporated directly with the food. For oral therapeutic administration, the active compounds may be incorporated with excipients and used in the form of ingestible tablets, buccal tablets, troches, capsules, elixirs, suspensions, syrups, wafers, and the like. The percentage of the compositions and preparations may, of course, be varied and may conveniently be between about 1 to about 80% of the weight of the unit. The amount of active compounds in such compositions is such that a suitable dosage can be obtained.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the active compounds in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. Dimethyl sulfoxide can be used as solvent for extremely rapid penetration, delivering high concentrations of the active agents to a small area.

The formulations of compounds can be presented in unit-dose or multi-dose sealed containers, such as ampules and vials. Thus, the composition can be in unit dosage form. In such form the preparation is subdivided into unit doses containing appropriate quantities of the active component. Thus, the compositions can be administered in a variety of unit dosage forms depending upon the method of administration. For example, unit dosage forms suitable for oral administration include, but are not limited to, powder, tablets, pills, capsules and lozenges.

In embodiments, the disclosure provides orally-administrable pharmaceutical compositions comprising an ITK inhibitor and a pharmaceutically acceptable excipient. In embodiments, the pharmaceutical composition is an oral composition. In embodiments, the oral composition is a solid oral composition. In embodiments, the oral composition is a liquid oral composition. In embodiments, the pharmaceutical composition is a tablet or a capsule. In embodiments, the pharmaceutical composition is a tablet. In embodiments, the pharmaceutical composition is a capsule. In embodiments, the pharmaceutical composition is a powder.

### Embodiments A1-A69

Embodiment A1. A method of treating a patient having deficient Th1 activity, the method comprising administering to the patient an ITK inhibitor at an effective amount to increase Th1 activity.

Embodiment A2. A method of treating a patient having deficient Th1 activity, the method comprising: (i) measuring increased Th2 activity, an increased level of IL-4, an increased level of IL-5, an increased level of IL-10, an increased level of IL-13, an increased level of IL-17, a decreased level of IFNγ, a decreased number of Th1⁺ T cells, a decreased ratio of Th1⁺ T cells to Th2⁺ T cells, a decreased ratio of IFNγ*CD4⁺ T cells to IL-4⁺CD4⁺ T cells, a decreased number CD8+ cytotoxic lymphocytes, an increased number of Th2+ cells, an increased number of Th17+ T cells, an increased number of eosinophils; or any combination of two or more thereof, relative to a control, in a biological sample obtained from the patient; and (ii) administering to the patient an ITK inhibitor at an effective amount to increase Th1 activity.

Embodiment A3. The method of Embodiment A1 or A2, wherein the patient has increased Th2 activity relative to a control.

Embodiment A4. The method of any one of Embodiments A1 to A3, wherein the patient has an increased level of a pro-inflammatory cytokine relative to a control.

Embodiment A5. The method of Embodiment A4, wherein the pro-inflammatory cytokine is IL-4, IL-5, IL-10, IL-13, IL-17, or a combination of two or more thereof.

Embodiment A6. The method of any one of Embodiments A1 to A5, wherein the effective amount to increase Th1 activity is an amount that: (a) increases the number of Th1⁺ T cells; (b) increases the ratio of Th1⁺ T cells to Th2⁺ T cells; (c) increases the ratio of IFNγ⁺CD4⁺ T cells to IL-4⁺CD4⁺ T cells; (d) increases IFNγ production; (e) increases CD8+ cytotoxic lymphocytes; (f) inhibits IL-4 production; (g) decreases Th17⁺ T cells; (h) decreases eosinophils; (i) a combination of two or more of (a)-(h).

Embodiment A7. The method of any one of Embodiments A1 to A5, wherein the effective amount to increase Th1 activity is an amount that: (a) increases the number of Th1⁺ T cells; (b) increases the ratio of Th1⁺ T cells to Th2⁺ T cells; (c) increases the ratio of IFNγ⁺CD4⁺ T cells to IL-4⁺CD4⁺ T cells; (d) increases IFNγ production; (e) increases CD8+ cytotoxic lymphocytes; (f) inhibits IL-4 production; (g) inhibits IL-13 production; (h) decreases Th2+ cells; (i) decreases Th17+ T cells; (j) decreases eosinophils; (k) a combination of two or more of (a)-(j).

Embodiment A8. The method of any one of Embodiments A1 to A7, wherein the effective amount to increase Th1 activity is an amount that inhibits production of cytokines secreted by Th2+ cells.

Embodiment A9. The method of any one of Embodiments A1 to A8, wherein the effective amount to increase Th1 activity is from about 0.6 mmole to about 1.6 mmole of the ITK inhibitor per day.

Embodiment A10. The method of Embodiment A9, wherein the effective amount to increase Th1 activity is about 0.6 mmole to about 1.0 mmole of the ITK inhibitor per day.

Embodiment A11. The method of Embodiment A10, wherein the effective amount to increase Th1 activity is about 0.7 mmole to about 0.9 mmole of the ITK inhibitor per day.

Embodiment A12. The method of Embodiment A11, wherein the effective amount to increase Th1 activity is about 0.8 mmole of the ITK inhibitor per day.

Embodiment A13. The method of any one of Embodiments A1 to A8, wherein the effective amount to increase Th1 activity is about 0.3 mmole to about 0.8 mmole of the ITK inhibitor twice per day.

Embodiment A14. The method of Embodiment A13, wherein the effective amount to increase Th1 activity is about 0.3 mmole to about 0.5 mmole of the ITK inhibitor twice per day.

Embodiment A15. The method of Embodiment A14, wherein the effective amount to increase Th1 activity is about 0.35 mmole to about 0.45 mmole of the ITK inhibitor twice per day.

Embodiment A16. The method of Embodiment A15, wherein the effective amount to increase Th1 activity is about 0.4 mmole of the ITK inhibitor twice per day.

Embodiment A17. A method of treating a cancer, an autoimmune disease, or an allergy in a patient in need thereof, the method comprising administering to the patient about 0.6 mmole per day to about 1.6 mmole per day of an ITK inhibitor.

Embodiment A18. A method of treating a cancer, an autoimmune disease, or an allergy in a patient in need thereof, the method comprising: (i) measuring a decreased level of Th1⁺ T cells, a decreased ratio of Th1⁺ T cells to Th2⁺ T cells; a decreased ratio of IFNγ⁺CD4⁺ T cells to IL-4⁺CD4⁺ T cells; a decreased level of IFNγ; a decreased level CD8+ cytotoxic lymphocytes; an increased level of Th2+ cells; an increased level of IL-4; an increased level of IL-5; an increased level of IL-10; an increased level of IL-13; an increased level of IL-17; an increased level of Th17+ T cells; an increased level of eosinophils; a decreased level of IL-1β, a decreased level of IL-2, a decreased level of IL-12, a decreased level of TNF-α, a decreased level of TNF-γ, a decreased level of GMCS, or a combination of two or more thereof, relative to a control, in a biological sample obtained from the subject, and (ii) administering to the patient about 0.6 mmole per day to about 1.6 mmole per day of an ITK inhibitor.

Embodiment A19. The method of Embodiment A17 or A18, wherein the cancer is T-cell lymphoma or T-cell leukemia.

Embodiment A20. The method of Embodiment A17 or A18, wherein the cancer is peripheral T-cell lymphoma.

Embodiment A21. The method of Embodiment A17 or A18, wherein the cancer is peripheral T-cell lymphoma not otherwise specified.

Embodiment A22. The method of Embodiment A17 or A18, wherein the cancer is cutaneous T-cell lymphoma.

Embodiment A23. The method of Embodiment A17 or A18, wherein the cancer is a solid tumor.

Embodiment A24. The method of Embodiment A17 or A18, wherein the cancer is lung cancer, colorectal cancer, pancreatic cancer, prostate cancer, breast cancer, gastric cancer, or head and neck cancer.

Embodiment A25. The method of Embodiment A17 or A18, wherein the cancer is lung cancer, colorectal cancer, pancreatic cancer, prostate cancer, breast cancer, gastric cancer, renal cancer, or head and neck cancer.

Embodiment A26. The method of any one of Embodiments A17 to A25, wherein the cancer is a relapsed/refractory cancer.

Embodiment A27. The method of Embodiment A17 or A18, wherein the autoimmune disease is autoimmune lymphoproliferative disease, ulcerative colitis, or systemic lupus erythematosus.

Embodiment A28. The method of Embodiment A17 or A18, wherein the autoimmune disease is autoimmune lymphoproliferative disease, colitis, inflammatory bowel disease, or systemic lupus erythematosus.

Embodiment A29. The method of Embodiment A17 or A18, wherein the allergy is asthma, dermatitis, rhinitis, or psoriasis.

Embodiment A30. A method of treating Th2/ITK-mediated disease in a patient in need thereof, the method comprising administering to the patient about 0.6 mmole per day to about 1.6 mmole per day of an ITK inhibitor.

Embodiment A31. A method of treating Th2/ITK-mediated disease in a patient in need thereof, the method comprising: (i) measuring a decreased level of Th1⁺ T cells, a decreased ratio of Th1⁺ T cells to Th2⁺ T cells; a decreased ratio of IFNγ*CD4⁺ T cells to IL-4⁺CD4⁺ T cells; a decreased level of IFNγ; a decreased level CD8+ cytotoxic lymphocytes; an increased level of Th2+ cells; an increased level of IL-4; an increased level of IL-5; an increased level of IL-10; an increased level of IL-13; an increased level of IL-17; an increased level of Th17+ T cells; an increased level of eosinophils; a decreased level of IL-1β, a decreased level of IL-2, a decreased level of IL-12, a decreased level of TNF-α, a decreased level of TNF-γ, a decreased level of GMCS, or a combination of two or more thereof, relative to a control, in a biological sample obtained from the subject, and (ii) administering to the patient about 0.6 mmole per day to about 1.6 mmole per day of an ITK inhibitor.

Embodiment A32. The method of Embodiment A30 or A31, wherein the Th2/ITK-mediated disease is atopic dermatitis, asthma, rhinitis, conjunctivitis, psoriasis, scleroderma, pulmonary fibrosis, cirrhosis, retroperitoneal fibrosis, psoriatic arthritis, vasculitis, autoimmune lymphoproliferative syndrome, chronic obstructive pulmonary disease, an eosinophilic disease, a mast cell disease, or human immunodeficiency viral disease.

Embodiment A33. The method of Embodiment A30 or A31, wherein the Th2/ITK-mediated disease is atopic dermatitis.

Embodiment A34. The method of Embodiment A30 or A31, wherein the Th2/ITK-mediated disease is asthma.

Embodiment A35. The method of Embodiment A30 or A31, wherein the Th2/ITK-mediated disease is rhinitis.

Embodiment A36. The method of Embodiment A30 or A31, wherein the Th2/ITK-mediated disease is conjunctivitis.

Embodiment A37. The method of Embodiment A30 or A31, wherein the Th2/ITK-mediated disease is psoriasis.

Embodiment A38. The method of Embodiment A30 or A31, wherein the Th2/ITK-mediated disease is a fibrotic disease.

Embodiment A39. The method of Embodiment A30 or A31, wherein the Th2/ITK-mediated disease is scleroderma.

Embodiment A40. The method of Embodiment A30 or A31, wherein the Th2/ITK-mediated disease is pulmonary fibrosis.

Embodiment A41. The method of Embodiment A40, wherein the pulmonary fibrosis is idiopathic pulmonary fibrosis.

Embodiment A42. The method of Embodiment A30 or A31, wherein the Th2/ITK-mediated disease is cirrhosis.

Embodiment A43. The method of Embodiment A30 or A31, wherein the Th2/ITK-mediated disease is retroperitoneal fibrosis. Th2/ITK-mediated disease.

Embodiment A44. The method of Embodiment A30 or A31, wherein the Th2/ITK-mediated disease is psoriatic arthritis.

Embodiment A45. The method of Embodiment A30 or A31, wherein the Th2/ITK-mediated disease is vasculitis.

Embodiment A46. The method of Embodiment A30 or A31, wherein the Th2/ITK-mediated disease is autoimmune lymphoproliferative syndrome.

Embodiment A47. The method of Embodiment A30 or A31, wherein the Th2/ITK-mediated disease is chronic obstructive pulmonary disease.

Embodiment A48. The method of Embodiment A30 or A31, wherein the Th2/ITK-mediated disease is an eosinophilic disease.

Embodiment A49. The method of Embodiment A48, wherein the eosinophilic disease is esophagitis.

Embodiment A50. The method of Embodiment A30 or A31, wherein the Th2/ITK-mediated disease is a mast cell disease.

Embodiment A51. The method of Embodiment A50, wherein the mast cell disease is mastocytosis, mast cell activation syndrome, or hereditary alpha tryptasemia.

Embodiment A52. The method of Embodiment A30 or A31, wherein the Th2/ITK-mediated disease is human immunodeficiency viral disease.

Embodiment A53. The method of any one of Embodiments A1 to A52, wherein the ITK inhibitor has a selectivity for ITK that is at least 50-fold greater than the selectivity for resting lymphocyte kinase.

Embodiment A54. The method of any one of Embodiments A1 to A52, wherein the ITK inhibitor has a selectivity for ITK that is at least 100-fold greater than the selectivity for resting lymphocyte kinase.

Embodiment A55. The method of any one of Embodiments A1 to A54, wherein the ITK inhibitor is a compound of Formula (A) or a pharmaceutically acceptable salt thereof:

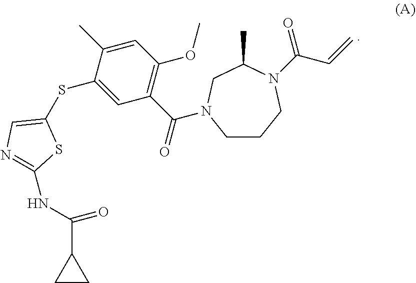 [SMILES: C=CC(=O)N1CCCN(C(=O)c2cc(Sc3cnc(NC(=O)C4CC4)s3)c(C)cc2OC)C[C@H]1C ; "."]

Embodiment A56. The method of any one of Embodiments A17 to A55, comprising administering to the patient about 0.6 mmole to about 1.2 mmole of the ITK inhibitor per day.

Embodiment A57. The method of Embodiment A56, comprising administering to the patient about 0.6 mmole to about 1.0 mmole of the ITK inhibitor per day.

Embodiment A58. The method of Embodiment A56, comprising administering to the patient about 0.7 mmole to about 0.9 mmole of the ITK inhibitor per day.

Embodiment A59. The method of Embodiment A56, comprising administering to the patient about 0.8 mmole of the ITK inhibitor per day.

Embodiment A60. The method of any one of Embodiments A17 to A55, comprising administering to the patient about 0.3 mmole to about 0.8 mmole of the ITK inhibitor twice per day.

Embodiment A61. The method of Embodiment A60, comprising administering to the patient about 0.3 mmole to about 0.5 mmole of the ITK inhibitor twice per day.

Embodiment A62. The method of Embodiment A60, comprising administering to the patient about 0.35 mmole to about 0.45 mmole of the ITK inhibitor twice per day.

Embodiment A63. The method of Embodiment A60, comprising administering to the patient about 0.4 mmole of the ITK inhibitor twice per day.

Embodiment A64. The method of any one of Embodiments A6-A16, A18-A29, and A31-A63, wherein (i) comprises measuring an increased level of Th2+ cells; an increased level of IL-4; an increased level of IL-5; an increased level of IL-10; an increased level of IL-13; an increased level of IL-17; or a combination of two or more thereof, relative to a control, in the biological sample obtained from the subject.

Embodiment A65. The method of Embodiment A64, wherein (i) comprises measuring an increased level of Th2+ cells, relative to a control, in the biological sample obtained from the subject.

Embodiment A66. The method of Embodiment A64, wherein (i) comprises measuring an increased level of IL-4; an increased level of IL-5; an increased level of IL-10; an increased level of IL-13; an increased level of IL-17; or a combination of two or more thereof, relative to a control, in a biological sample obtained from the subject.

Embodiment A67. The method of any one of Embodiments A1 to A66, comprising the steps, in order: (i) administering the ITK inhibitor for about 4 weeks to about 12 weeks; (ii) discontinuing administration of the ITK inhibitor for about 1 week to about 8 weeks, and (iii) administering the ITK inhibitor for at least four weeks.

Embodiment A68. The method of any one of Embodiments A1 to A66, comprising the steps, in order: (i) administering the ITK inhibitor for about 4 weeks to about 12 weeks; (ii) discontinuing administration of the ITK inhibitor for about 1 week to about 8 weeks, and (iii) administering the ITK inhibitor for about 4 weeks to about 12 weeks.

Embodiment A69. The method of Embodiment A67 or A68, further comprising repeating steps (ii) and (iii).

### Embodiments B1-B68

Embodiment B1. A method for treating a cancer, an autoimmune disease, or an allergy in a patient in need thereof, the method comprising administering to the patient about 250 mg to about 1,000 mg per day of a compound of Formula (A) or a pharmaceutically acceptable salt thereof, wherein the compound of Formula (A) is:

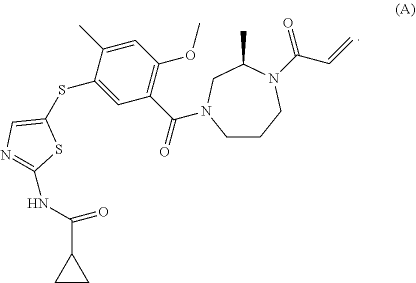 [SMILES: C=CC(=O)N1CCCN(C(=O)c2cc(Sc3cnc(NC(=O)C4CC4)s3)c(C)cc2OC)C[C@H]1C ; "."]

Embodiment B2. The method of Embodiment B1 for treating the cancer.

Embodiment B3. The method of Embodiment B2, wherein the cancer is lymphoma.

Embodiment B4. The method of Embodiment B3, wherein the lymphoma is T-cell lymphoma.

Embodiment B5. The method of Embodiment B3, wherein the lymphoma is peripheral T-cell lymphoma.

Embodiment B6. The method of Embodiment B3, wherein the lymphoma is peripheral T-cell lymphoma not otherwise specified.

Embodiment B7. The method of Embodiment B3, wherein the lymphoma is cutaneous T-cell lymphoma.

Embodiment B8. The method of Embodiment B2, wherein the cancer is a solid tumor.

Embodiment B9. The method of Embodiment B2, wherein the cancer is lung cancer, colorectal cancer, pancreatic cancer, prostate cancer, breast cancer, gastric cancer, or head and neck cancer.

Embodiment B10. The method of Embodiment B2, wherein the cancer is lung cancer, colorectal cancer, pancreatic cancer, prostate cancer, breast cancer, gastric cancer, renal cancer, or head and neck cancer.

Embodiment B11. The method of Embodiment B2, wherein the cancer is leukemia.

Embodiment B12. The method of Embodiment B11, wherein the leukemia is T-cell leukemia.

Embodiment B13. The method of any one of Embodiments B1 to B12, wherein the cancer is a relapsed/refractory cancer.

Embodiment B14. The method of Embodiment B1 for treating the autoimmune disease.

Embodiment B15. The method of Embodiment B14, wherein the autoimmune disease is autoimmune lymphoproliferative disease.

Embodiment B16. The method of Embodiment B14, wherein the autoimmune disease is colitis.

Embodiment B17. The method of Embodiment B16, wherein the colitis is ulcerative colitis.

Embodiment B18. The method of Embodiment B14, wherein the autoimmune disease is inflammatory bowel disease.

Embodiment B19. The method of Embodiment B14, wherein the autoimmune disease is systemic lupus erythematosus.

Embodiment B20. The method of Embodiment B1 for treating the allergy.

Embodiment B21. The method of Embodiment B20, wherein the allergy is asthma, dermatitis, rhinitis, or psoriasis.

Embodiment B22. The method of Embodiment B20, wherein the allergy is allergic asthma, atopic dermatitis, allergic dermatitis, allergic rhinitis, or psoriasis.

Embodiment B23. A method for an Th2/ITK-mediated disease in a patient in need thereof, the method comprising administering to the patient about 250 mg to about 1,000 mg per day of a compound of Formula (A) or a pharmaceutically acceptable salt thereof, wherein the compound of Formula (A) is:

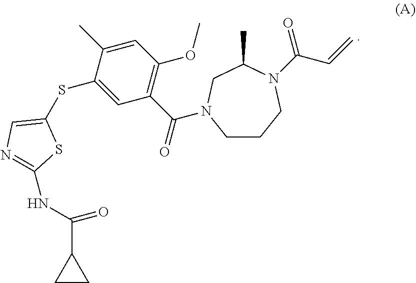 [SMILES: C=CC(=O)N1CCCN(C(=O)c2cc(Sc3cnc(NC(=O)C4CC4)s3)c(C)cc2OC)C[C@H]1C ; "."]

Embodiment B24. The method of Embodiment B23, wherein the Th2/ITK-mediated disease is atopic dermatitis, asthma, rhinitis, conjunctivitis, psoriasis, scleroderma, pulmonary fibrosis, cirrhosis, retroperitoneal fibrosis, psoriatic arthritis, vasculitis, autoimmune lymphoproliferative syndrome, chronic obstructive pulmonary disease, an eosinophilic disease, a mast cell disease, or human immunodeficiency viral disease.

Embodiment B25. The method of Embodiment B23, wherein the Th2/ITK-mediated disease is atopic dermatitis.

Embodiment B26. The method of Embodiment B23, wherein the Th2/ITK-mediated disease is asthma.

Embodiment B27. The method of Embodiment B23, wherein the Th2/ITK-mediated disease is rhinitis.

Embodiment B28. The method of Embodiment B23, wherein the Th2/ITK-mediated disease is conjunctivitis.

Embodiment B29. The method of Embodiment B23, wherein the Th2/ITK-mediated disease is psoriasis.

Embodiment B30. The method of Embodiment B23, wherein the Th2/ITK-mediated disease is a fibrotic disease.

Embodiment B31. The method of Embodiment B23, wherein the Th2/ITK-mediated disease is scleroderma.

Embodiment B32. The method of Embodiment B23, wherein the Th2/ITK-mediated disease is pulmonary fibrosis.

Embodiment B33. The method of Embodiment B32, wherein the pulmonary fibrosis is idiopathic pulmonary fibrosis.

Embodiment B34. The method of Embodiment B23, wherein the Th2/ITK-mediated disease is cirrhosis.

Embodiment B35. The method of Embodiment B23, wherein the Th2/ITK-mediated disease is retroperitoneal fibrosis.

Embodiment B36. The method of Embodiment B23, wherein the Th2/ITK-mediated disease is psoriatic arthritis.

Embodiment B37. The method of Embodiment B23, wherein the Th2/ITK-mediated disease is vasculitis.

Embodiment B38. The method of Embodiment B23, wherein the Th2/ITK-mediated disease is autoimmune lymphoproliferative syndrome.

Embodiment B39. The method of Embodiment B23, wherein the Th2/ITK-mediated disease is chronic obstructive pulmonary disease.

Embodiment B40. The method of Embodiment B23, wherein the Th2/ITK-mediated disease is an eosinophilic disease.

Embodiment B41. The method of Embodiment B40, wherein the eosinophilic disease is esophagitis.

Embodiment B42. The method of Embodiment B23, wherein the Th2/ITK-mediated disease is a mast cell disease.

Embodiment B43. The method of Embodiment B42, wherein the mast cell disease is mastocytosis, mast cell activation syndrome, or hereditary alpha tryptasemia.

Embodiment B44. The method of Embodiment B23, wherein the Th2/ITK-mediated disease is human immunodeficiency viral disease.

Embodiment B45. The method of any one of Embodiments B1 to B44, comprising administering to the patient about 250 mg to about 900 mg per day of the compound of Formula (A) or the pharmaceutically acceptable salt thereof.

Embodiment B46. The method of Embodiment B45, comprising administering to the patient about 250 mg to about 800 mg per day of the compound of Formula (A) or the pharmaceutically acceptable salt thereof.

Embodiment B47. The method of Embodiment B45, comprising administering to the patient about 250 mg to about 700 mg per day of the compound of Formula (A) or the pharmaceutically acceptable salt thereof.

Embodiment B48. The method of Embodiment B45, comprising administering to the patient about 250 mg to about 600 mg per day of the compound of Formula (A) or the pharmaceutically acceptable salt thereof.

Embodiment B49. The method of Embodiment B45, comprising administering to the patient about 250 mg to about 550 mg per day of the compound of Formula (A) or the pharmaceutically acceptable salt thereof.

Embodiment B50. The method of Embodiment B45, comprising administering to the patient about 300 mg to about 500 mg per day of the compound of Formula (A) or the pharmaceutically acceptable salt thereof.

Embodiment B51. The method of Embodiment B45, comprising administering to the patient about 350 mg to about 450 mg per day of the compound of Formula (A) or the pharmaceutically acceptable salt thereof.

Embodiment B52. The method of Embodiment B45, comprising administering to the patient about 400 mg per day of the compound of Formula (A) or the pharmaceutically acceptable salt thereof.

Embodiment B53. The method of any one of Embodiments B1 to B44, comprising administering to the patient about 125 mg to about 500 mg twice per day per day of the compound of Formula (A) or the pharmaceutically acceptable salt thereof.

Embodiment B54. The method of Embodiment B53, comprising administering to the patient about 125 mg to about 450 mg twice per day of the compound of Formula (A) or the pharmaceutically acceptable salt thereof.

Embodiment B55. The method of Embodiment B53, comprising administering to the patient about 125 mg to about 400 mg twice per day of the compound of Formula (A) or the pharmaceutically acceptable salt thereof.

Embodiment B56. The method of Embodiment B53, comprising administering to the patient about 125 mg to about 350 mg twice per day of the compound of Formula (A) or the pharmaceutically acceptable salt thereof.

Embodiment B57. The method of Embodiment B53, comprising administering to the patient about 125 mg to about 300 mg twice per day of the compound of Formula (A) or the pharmaceutically acceptable salt thereof.

Embodiment B58. The method of Embodiment B53, comprising administering to the patient about 125 mg to about 275 mg twice per day of the compound of Formula (A) or the pharmaceutically acceptable salt thereof.

Embodiment B59. The method of Embodiment B53, comprising administering to the patient about 150 mg to about 250 mg twice per day of the compound of Formula (A) or the pharmaceutically acceptable salt thereof.

Embodiment B60. The method of Embodiment B53, comprising administering to the patient about 175 mg to about 225 mg twice per day of the compound of Formula (A) or the pharmaceutically acceptable salt thereof.

Embodiment B61. The method of Embodiment B53, comprising administering to the patient about 200 mg twice per day of the compound of Formula (A) or the pharmaceutically acceptable salt thereof.

Embodiment B62. The method of any one of Embodiments B1 to B61, comprising the steps, in order: (i) administering the compound of Formula (A) or the pharmaceutically acceptable salt thereof for about 4 weeks to about 12 weeks; (ii) discontinuing administration of the compound of Formula (A) or the pharmaceutically acceptable salt thereof for about 1 week to about 8 weeks, and (iii) administering the compound of Formula (A) or the pharmaceutically acceptable salt thereof for at least 4 weeks.

Embodiment B63. The method of any one of Embodiments B1 to B61, comprising the steps, in order: (i) administering the compound of Formula (A) or the pharmaceutically acceptable salt thereof for about 4 weeks to about 12 weeks; (ii) discontinuing administration of the compound of Formula (A) or the pharmaceutically acceptable salt thereof for about 1 week to about 8 weeks, and (iii) administering the compound of Formula (A) or the pharmaceutically acceptable salt thereof for about 4 weeks to about 12 weeks.

Embodiment B64. The method of Embodiment B62 or B63, further comprising repeating steps (ii) and (iii).

Embodiment B65. The method of any one of Embodiments B1 to B64, further comprising; prior to administering to the patient the compound of Formula (A) or the pharmaceutically acceptable salt thereof; measuring a decreased level of Th1⁺ T cells, a decreased ratio of Th1⁺ T cells to Th2⁺ T cells; a decreased ratio of IFNγ⁺CD4⁺ T cells to IL-4⁺CD4⁺ T cells; a decreased level of IFNγ; a decreased level CD8⁺ cytotoxic lymphocytes; an increased level of Th2⁺ cells; an increased level of IL-4; an increased level of IL-5; an increased level of IL-10; an increased level of IL-13; an increased level of IL-17; an increased level of Th17+ T cells; an increased level of eosinophils; a decreased level of IL-1β, a decreased level of IL-2, a decreased level of IL-12, a decreased level of TNF-α, a decreased level of TNF-γ, a decreased level of GMCS, or a combination of two or more thereof, relative to a control, in a biological sample obtained from the subject.

Embodiment B66. The method of any one of Embodiments B1 to B64, further comprising; prior to administering to the patient the compound of Formula (A) or the pharmaceutically acceptable salt thereof; measuring an increased level of Th2+ cells; an increased level of IL-4; an increased level of IL-5; an increased level of IL-10; an increased level of IL-13; an increased level of IL-17; or a combination of two or more thereof, relative to a control, in a biological sample obtained from the subject,

Embodiment B67. The method of any one of Embodiments B1 to B64, further comprising; prior to administering to the patient the compound of Formula (A) or the pharmaceutically acceptable salt thereof; measuring an increased level of IL-4; an increased level of IL-5; an increased level of IL-10; an increased level of IL-13; an increased level of IL-17; or a combination of two or more thereof, relative to a control, in a biological sample obtained from the subject.

Embodiment B68. The method of any one of Embodiments B1 to B64, further comprising; prior to administering to the patient the compound of Formula (A) or the pharmaceutically acceptable salt thereof; measuring an increased level of Th2+ cells, relative to a control, in a biological sample obtained from the subject.

### Embodiments C1-C4

Embodiment C1. A pharmaceutical composition comprising about 150 mg to about 250 mg of a compound of Formula (A) or a pharmaceutically acceptable salt thereof and a pharmaceutically acceptable excipient, wherein the compound of Formula (A) is:

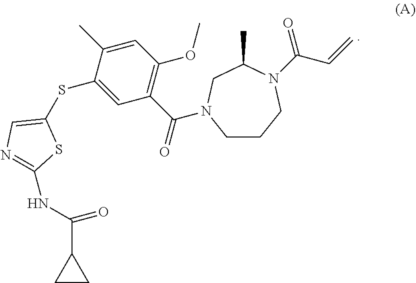 [SMILES: C=CC(=O)N1CCCN(C(=O)c2cc(Sc3cnc(NC(=O)C4CC4)s3)c(C)cc2OC)C[C@H]1C ; "."]

Embodiment C2. The pharmaceutical composition of Embodiment 125, comprising about 200 mg of the compound of Formula (A) or the pharmaceutically acceptable salt thereof.

Embodiment C3. A pharmaceutical composition comprising about 300 mg to about 500 mg of a compound of Formula (A) or a pharmaceutically acceptable salt thereof and a pharmaceutically acceptable excipient, wherein the pharmaceutical composition is in a single or divided dose, and wherein the compound of Formula (A) or the pharmaceutically acceptable salt thereof is:

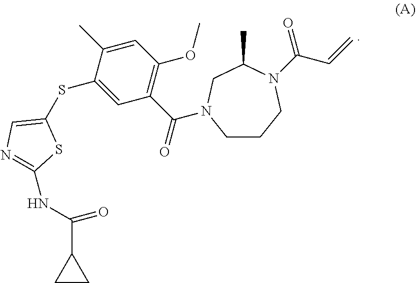 [SMILES: C=CC(=O)N1CCCN(C(=O)c2cc(Sc3cnc(NC(=O)C4CC4)s3)c(C)cc2OC)C[C@H]1C ; "."]

Embodiment C4. The pharmaceutical composition of Embodiment 127, comprising about 400 mg of the compound of Formula (A) or the pharmaceutically acceptable salt thereof, wherein the pharmaceutical composition is in a single or divided dose.

### Embodiments D1-D53

Embodiment D1. A method of reversing T cell exhaustion in a patient in need thereof, the method comprising administering to the patient an effective amount of an ITK inhibitor.

Embodiment D2. A method of reversing T cell exhaustion in a patient in need thereof, the method comprising: (i) measuring an increased level of LAG3, an increased level of TIGIT, an increased level of PD-1, or a combination of two or more thereof, relative to a control, in a biological sample obtained from the patient; and (ii) administering to the patient an effective amount of an ITK inhibitor.

Embodiment D3. A method of reversing T cell exhaustion in a patient in need thereof, the method comprising: (i) measuring a decreased level of IFN, a decreased level of granzyme B, or a combination of two or more thereof, relative to a control, in a biological sample obtained from the patient; and (ii) administering to the patient an effective amount of an ITK inhibitor.

Embodiment D4. A method of reversing T cell exhaustion in a patient in need thereof, the method comprising: (i) measuring an increased level of LAG3, an increased level of TIGIT, an increased level of PD-1, a decreased level of IFN, a decreased level of granzyme B, or a combination of two or more thereof, relative to a control, in a biological sample obtained from the patient; and (ii) administering to the patient an effective amount of an ITK inhibitor.

Embodiment D5. The method of any one of Embodiments D1 to D4, wherein the T cell is a CD4 T cell.

Embodiment D6. The method of any one of Embodiments D1 to D5, wherein the patient has cancer.

Embodiment D7. A method of treating cancer in a patient in need thereof, the method comprising administering to the patient an effective amount of an ITK inhibitor; wherein the patient has T cell exhaustion.

Embodiment D8. A method of treating cancer in a patient in need thereof, the method comprising (i) measuring an increased level of LAG3, TIGIT, PD-1, or a combination of two or more thereof, relative to a control, in a biological sample obtained from the patient; and (ii) administering to the patient an effective amount of an ITK inhibitor.

Embodiment D9. A method of treating cancer in a patient in need thereof, the method comprising (i) measuring a decreased level of IFN, a decreased level of granzyme B or a combination of two or more thereof, relative to a control, in a biological sample obtained from the patient; and (ii) administering to the patient an effective amount of an ITK inhibitor.

Embodiment D10. A method of treating cancer in a patient in need thereof, the method comprising (i) measuring an increased level of LAG3, an increased level of TIGIT, an increased level of PD-1, a decreased level of IFN, a decreased level of granzyme B, or a combination of two or more thereof, relative to a control, in a biological sample obtained from the patient; and (ii) administering to the patient an effective amount of an ITK inhibitor.

Embodiment D11. The method of any one of Embodiments D1 to D10, wherein the method increases the level of IFNγ; increases the level of granzyme B; or increases the level of IFNγ and granzyme B, relative to a control or relative to the level prior to administration of the ITK inhibitor.

Embodiment D12. The method of any one of Embodiments D1 to D10, wherein the method decreases the level of LAG3, decreases the level of TIGIT, decreases the level of PD-1, or the combination of two or more thereof, relative to the level of LAG3, TIGIT, PD-1, or the combination of two or more thereof, relative to a control or relative to the level prior to administration of the ITK inhibitor.

Embodiment D13 The method of any one of Embodiments D1 to D10, wherein the method increases the level of IFNγ; increases the level of granzyme B; decreases the level of LAG3, decreases the level of TIGIT, decreases the level of PD-1, or a combination of two or more of the foregoing, relative to the level of IFNγ, granzyme B, LAG3, TIGIT, PD-1, or the combination of two or more thereof, respectively, relative to a control or relative to the level prior to administration of the ITK inhibitor.

Embodiment D14. The method of any one of Embodiments D6 to D13, wherein the cancer is lymphoma.

Embodiment D15. The method of Embodiment D14, wherein the lymphoma is T-cell lymphoma.

Embodiment D16. The method of Embodiment D14, wherein the lymphoma is peripheral T-cell lymphoma.

Embodiment D17. The method of Embodiment D14, wherein the lymphoma is peripheral T-cell lymphoma not otherwise specified.

Embodiment D18. The method of Embodiment D14, wherein the lymphoma is cutaneous T-cell lymphoma.

Embodiment D19. The method of any one of Embodiments D6 to D13, wherein the cancer is a solid tumor.

Embodiment D20. The method of any one of Embodiments D6 to D13, wherein the cancer is lung cancer, colorectal cancer, pancreatic cancer, prostate cancer, breast cancer, gastric cancer, renal cancer, or head and neck cancer.

Embodiment D21. The method of any one of Embodiments D6 to D13, wherein the cancer is leukemia.

Embodiment D22. The method of Embodiment D21, wherein the leukemia is T-cell leukemia.

Embodiment D23. The method of any one of Embodiments D6 to D22, wherein the cancer is a relapsed/refractory cancer.

Embodiment D24. The method of any one of Embodiments D1 to D23, wherein the effective amount is from about 250 mg to about 1,000 mg per day.

Embodiment D25. The method of Embodiment D24, wherein the effective amount is from about 250 mg to about 900 mg per day.

Embodiment D26. The method of Embodiment D24, wherein the effective amount is from about 250 mg to about 800 mg per day.

Embodiment D27. The method of Embodiment D24, wherein the effective amount is from about 250 mg to about 700 mg per day.

Embodiment D28. The method of Embodiment D24, wherein the effective amount is from about 250 mg to about 600 mg per day.

Embodiment D29. The method of Embodiment D24, wherein the effective amount is from about 250 mg to about 550 mg per day.

Embodiment D30. The method of Embodiment D24, wherein the effective amount is from about 300 mg to about 500 mg per day.

Embodiment D31. The method of Embodiment D24, wherein the effective amount is from about 350 mg to about 450 mg per day.

Embodiment D32. The method of Embodiment D24, wherein the effective amount is about 400 mg per day.

Embodiment D33. The method of any one of Embodiments D1 to D23, wherein the effective amount is from about 125 mg to about 500 mg twice per day per day.

Embodiment D34. The method of Embodiment D33, wherein the effective amount is from about 125 mg to about 450 mg twice per day.

Embodiment D35. The method of Embodiment D33, wherein the effective amount is from about 125 mg to about 400 mg twice per day.

Embodiment D36. The method of Embodiment D33, wherein the effective amount is from about 125 mg to about 350 mg twice per day.

Embodiment D37. The method of Embodiment D33, wherein the effective amount is from about 125 mg to about 300 mg twice per day.

Embodiment D38. The method of Embodiment D33, wherein the effective amount is from about 125 mg to about 275 mg twice per day.

Embodiment D39. The method of Embodiment D33, wherein the effective amount is from about 150 mg to about 250 mg twice per day.

Embodiment D40. The method of Embodiment D33, wherein the effective amount is from about 175 mg to about 225 mg twice per day.

Embodiment D41. The method of Embodiment D33, wherein the effective amount is about 200 mg twice per day.

Embodiment D42. The method of any one of Embodiments D1 to D23, wherein the effective amount is from about 0.6 mmole to about 1.6 mmole per day.

Embodiment D43. The method of Embodiment D42, wherein the effective amount is from about 0.6 mmole to about 1.0 mmole per day.

Embodiment D44. The method of Embodiment D42, wherein the effective amount is from about 0.7 mmole to about 0.9 mmole per day.

Embodiment D45. The method of Embodiment D42, wherein the effective amount is about 0.8 mmole per day.

Embodiment D46. The method of any one of Embodiments D1 to D23, wherein the effective amount is from about 0.3 mmole to about 0.8 mmole twice per day.

Embodiment D47. The method of Embodiment D46, wherein the effective amount is from about 0.3 mmole to about 0.5 mmole twice per day.

Embodiment D48. The method of Embodiment D46, wherein the effective amount is from about 0.35 mmole to about 0.45 mmole twice per day.

Embodiment D49. The method of Embodiment D46, wherein the effective amount is about 0.4 mmole twice per day.

Embodiment D50. The method of any one of Embodiments D1 to D49, wherein the ITK inhibitor is a compound of Formula (A) or a pharmaceutically acceptable salt thereof:

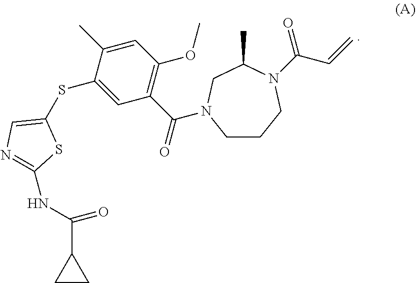 [SMILES: C=CC(=O)N1CCCN(C(=O)c2cc(Sc3cnc(NC(=O)C4CC4)s3)c(C)cc2OC)C[C@H]1C ; "."]

Embodiment D51. The method of any one of Embodiments D1 to D50, further comprising the steps, in order: (a) discontinuing administration of the ITK inhibitor for about 1 week to about 8 weeks, and (b) administering the ITK inhibitor for at least 4 weeks.

Embodiment D52. The method of any one of Embodiments D1 to D50, further comprising the steps, in order: (a) discontinuing administration of the ITK inhibitor for about 1 week to about 8 weeks, and (b) administering the ITK inhibitor for about 4 weeks to about 12 weeks.

Embodiment D53. The method of Embodiment D51 or D52, further comprising repeating steps (a) and (b).

### Embodiment E1-E2

Embodiment E1. The method of any one of Embodiments A1-A54, A56-A69, D1-D49, and D51-D53, wherein wherein the ITK inhibitor is a compound of Formula (I) or a pharmaceutically acceptable salt thereof, having the formula:

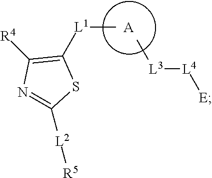

wherein Ring A is

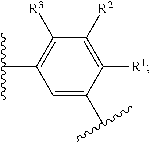

R¹ is independently hydrogen, halogen, —CX¹₃, —CHX¹₂, —CH₂X¹, —OCX¹₃, —OCH₂X¹, —OCHX², —CN, —SOₙ₁R^{1D}, —SOᵥ₁NR^{1A}R^{1B}, —NHC(O)NR^{1A}R^{1B}, —N(O)^{m1}, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² is independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³ is independently hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁴ is independently hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁵ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L¹ is —O—, —S—, or substituted or unsubstituted C₁-C₂ alkylene, or substituted or unsubstituted 2 membered heteroalkylene; L² is a bond, —NH—, or —NHC(O)—; L³ is a bond, —S(O)₂—, —N(R⁶)—, —O—, —S—, —C(O)—, —C(O)N(R⁶)—, —N(R⁶)C(O)—, —N(R⁶)C(O)NH—, —NHC(O)N(R⁶)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁶ is independently hydrogen, —CX⁶₃, —CHX⁶₂, —CH₂X⁶, —CN, —C(O)R^{6C}, —C(O)OR^{6C}, —C(O)NR^{6A}R^{6B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L⁴ is substituted or unsubstituted heterocycloalkylene; E is:

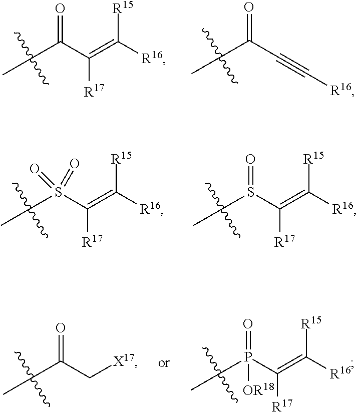

each R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{6A}, R^{6B} and R^{6C} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{6A}, and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; each X, X¹, X², X³, X⁴, and X⁶ is independently —F, —Cl, —Br, or —I; n1, n2, n3, and n4 are independently an integer from 0 to 2; m1, m2, m3, m4, v1, v2, v3, and v4 are independently 1 to 2; R¹⁵ is independently hydrogen, halogen, —CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R¹⁶ is independently hydrogen, halogen, —CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC═(O)NHNR^{16A}R^{16B}, NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16Λ}SO₂R^{16D}, —NR^{16Λ}C(O)R^{16C}, —NR^{16Λ}C(O)OR^{16C}, —NR^{16Λ}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R¹⁷ is independently hydrogen, halogen, —CX¹⁷₃, —CHX¹⁷₂, —CH₂X¹⁷, —CN, —SOₙ₁₇R^{17D}, SOᵥ₁₇NR^{17A}R^{17B}, —NHNR^{17A}R^{17B}, ONR^{17A}R^{17B}, —NHC═(O)NHNR^{17A}R^{17B}, —NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; R^{15Λ}, R^{15B}, R^{15C}, R^{15D}, R^{16A}, R^{16B}, R^{16C}, R^{16D}, R^{17A}, R^{17B}, R^{17C}, R^{17D}, R^{15A}, R^{18B}, and R^{18C} are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl; each X, X¹⁵, X¹⁶, X¹⁷ and X¹⁸ is independently —F, —Cl, —Br, or —I; n15, n16, and n17 are independently an integer from 0 to 2; v15, v16, and v17 are independently 1 or 2; and m15, m16, and m17 are independently 1 or 2.

Embodiment E2. The method of any one of Embodiments A1-A54, A56-A69, D1-D49, and D51-D53, wherein wherein the ITK inhibitor is a compound of Formula (II) or a pharmaceutically acceptable salt thereof, having the formula:

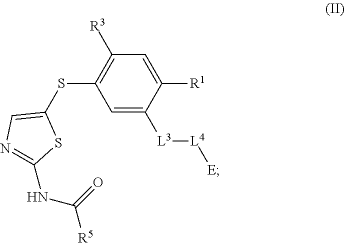

wherein: R¹ is hydrogen, halogen, —CX¹₃, —CHX¹₂, —CH₂X¹, —OCX¹₃, —OCH₂X¹, —OCHX¹₂, —CN, —SOₙ₁NR^{1D}, SOᵥ₁NR^{1A}R^{1B}, NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³ is hydrogen, halogen, —CX³₃, —CHX³₂, —CH₂X³, —OCX³₃, —OCH₂X³, —OCHX³₂, —CN, —SO₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, unsubstituted or substituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁵ is unsubstituted or substituted cycloalkyl, substituted or unsubstituted alkyl, substituted or unsubstituted heteroalkyl, substituted or unsubstituted heterocycloalkyl, substituted or unsubstituted aryl, or substituted or unsubstituted heteroaryl; L³ is —C(O)—, a bond, —N(R⁶)—, or —C(O)N(R⁶)—; R⁶ is hydrogen or methyl; L⁴ is substituted or unsubstituted 5 to 8 membered monocyclic heterocycloalkylene; E is —C(O)CH═CH₂; R^{1A}, R^{1B}, R^{1C}, R^{1D}, R^{3A}, R^{3B}, R^{3C}, and R^{3D} are each independently hydrogen, —CX₃, —CN, —COOH, —CONH₂, —CHX₂, —CH₂X, unsubstituted alkyl, unsubstituted heteroalkyl, unsubstituted cycloalkyl, unsubstituted heterocycloalkyl, unsubstituted aryl, or unsubstituted heteroaryl; X, X¹, and X³ are each independently —F, —Cl, —Br, or —I; n1 and n3 are independently an integer from 0 to 4; and m1, m3, v1, and v3 are independently 1 or 2.

### EXAMPLES

The following examples are for purposes of illustration and are not intended to limit the spirit or scope of the disclosure or claims.

### Example 1

The specific role of ITK in T cells suggests a utility for selective pharmacologic ITK inhibition for diseases driven by T cell activation, expansion and differentiation, including lupus, colitis, and T cell malignancy. However, data from knockout mouse studies do not distinguish between effects on T cell development and effects on TCR signaling in mature cells. To better understand the effects of selective inhibition of ITK on T cell signaling and immune function, we developed CPI-818, an irreversible small molecule inhibitor of ITK which possesses a high degree of selectivity for ITK over RLK and the other TEC-family kinases. Covalent binding of CPI-818 to Cys 442 of ITK was demonstrated by mass spectrometry and by competitive probe binding. Following T cell activation, CPI-818 inhibited the phosphorylation of the ITK substrate PLC γl (Y783) and downstream signaling molecules, and blocked IL-2 secretion. CPI-818 was found to be efficacious in the T cell adoptive transfer model of colitis, a model driven by Th1-driven IFNγ. So, while selective inhibition of ITK by CPI-818 alters TCR-driven signaling resulting in increased Th1 polarization, the overall decrease in the Th1 phenotype was sufficient to ameliorate colitis disease progression. CPI-818 has recently entered a Phase I clinical trial in patients with T cell lymphoma (NCT03952078).

### Experimental Procedures

### Biochemical Kinase Assays

Two biochemical assays of kinase activity were employed. The first is based on the Invitrogen Lanthascreen platform while the second used the PerkinElmer microfluidic based LabChip 3000 system. In the Lanthascreen assay each kinase (1.0 nM) was incubated with compound for 120 minutes at 22° C. in a buffer composed of 50 mM HEPES (pH 7.5), 10 mM MgCl₂, 1 mM EGTA, and 0.01% Brij 35. The kinase reaction was initiated by the addition of ATP (50 μM final) and fluorescein-poly-Glu-Tyr peptide (200 nM final). The reaction was quenched after 60 minutes at 22° C., and phospho-peptide product detected by the addition of EDTA (10 mM final) and terbium PY20 antibody (2 nM final). The phospho-peptide product was measured by time-resolved fluorescence resonance energy transfer (TR FRET) between the terbium of the antibody and fluorescein of the phospho-peptide using an EnVision plate reader (PerkinElmer, Waltham, MA) equipped to detect the TR-FRET signal (excitation 320 nm, dual emission at 495 nm and 520 nm). All reagents including recombinant human kinases, fluorescein-poly-Glu-Tyr peptide, terbium PY20 antibody, and kinase buffer A were purchased from Invitrogen.

Alternatively, in the microfluidic assay each kinase (0.2 nM) was incubated with compound for 15 minutes at 22° C. in a buffer composed of 100 mM HEPES (pH 7.5), 0.10% BSA, 0.01% Triton X-100, 1 mM DTT, 5 mM MgCl₂, 10 μM Sodium Orthovanadate, 10 μM Beta-Glycerophosphate and 1% DMSO. The kinase reaction was initiated by the addition of ATP (10 μM final) and fluorescein labeled Scrtide peptide (1 μM final). The reaction was quenched after 3 hours at 22° C. by the addition of EDTA (20 mM final), and phospho-peptide product detected by direct fluorescence (ex: 480 nm, em: 520 nm) after electrophoretic separation from the substrate using a PerkinElmer EZ reader. All reagents were purchased from Sigma-Aldrich.

Inhibition of kinase activity was measured by construction of a concentration response curve of the inhibitor. Controls were included containing no inhibitor (positive control) or a saturating concentration of a reference inhibitor providing complete inhibition (negative control). Both assay formats gave linear progress curves over the time course of the positive control reactions. The response reading was normalized according to equation 1 and plotted against the Log base 10 transformed values of the molar concentration of inhibitor. The IC₅₀ value was obtained by fitting equation 2 to the data. Technical duplicates of the concentration response were collected at each determination. Inhibition constants for all compounds were determined by the Lanthascreen assay except for CP-1651 and CP-2231 which were measured using the microfluidic assay.

$$Equations ( 1 ) and ( 2 )$$

$$Normalized Sample Value = \frac{Positive - Sample}{Positive - Negative} \times 100 \qquad ( 1 )$$

$$Y = \frac{100}{1 + {10}^{( ( Log ( {IC}_{50} ) - ( I ) ) \times C )}} \qquad ( 2 )$$

With reference to Equation (2): Y is the normalized response, (I) is the compound concentration, IC₅₀ is the compound concentration giving 50% inhibition and C is the slope of the curve.

### Dialysis Assay

Test compound (or DMSO control) was incubated at 20-fold the IC₅₀ value measured at the K_{M} for ATP with 20 nM ITK for one hour in a buffer composed of 100 mM HEPES (pH7.5), 0.1% BSA, 5 mM MgCl₂, 1 mM DTT, 0.01% Triton X-100. Dialysis was performed at room temperature against the same buffer for 24 hours in a Slide-A-Lyzer dialysis device (10 kDa MWCO) with three changes of the dialysis buffer giving a nominal final dialysis factor of 8×10⁶. Un-dialyzed control samples incubated in parallel for 24 hours. ITK activity was measured following dialysis in a continuous sampling format using the microfluidic assay and 300 μM ATP and 2 μM Scrtide peptide substrate. The enzymatic velocity of treated samples was normalized to the appropriate control rate and expressed as a percent.

### Competitive Tracer-Binding Assay

The assay is based on the rapid and reversible binding of the Alexa Fluor 647-labeled, ATP-competitive kinase inhibitor, tracer 236, to recombinant tagged forms of the human kinases ITK and RLK. Binding of the tracer to a kinase is detected using a europium-labeled anti-tag antibody where simultaneous binding of both the tracer and antibody to the kinase results in a high degree of FRET. An inhibitor to the kinase competes for binding with the tracer, resulting in a loss of FRET. The assay can be performed in a continuous mode enabling the collection of progress curves and measurement of binding kinetics. All reagents including GST-tagged recombinant human ITK (PV3875), GST-tagged recombinant human RLK (PV5860), Kinase tracer 236 (PV5592), Lanthascreen Eu-Anti-GST antibody (PV5594), 5× kinase buffer A (PV3189) were purchased from Invitrogen. A 14-point serial dilution of compound was prepared in 100% DMSO and subsequently diluted 33-fold in kinase buffer A to provide and intermediate solution of inhibitor. Kinase and Eu-anti-GST antibody were combined in kinase buffer A at a concentration of 15 nM and incubated for 10 min at room temperature. This solution was diluted with an equal volume 120 nM tracer 236 in kinase buffer A to yield the assembled kinase complex solution which was then incubated for 20 min at room temperature. The assay was initiated by the addition of 10 μL of assembled kinase complex solution to 5 μL of intermediate inhibitor solution. Data collection began immediately after assay initiation using an EnVision plate reader (excitation 340 nm, dual emission at 615 nm and 665 nm) at 1 min intervals for 2 h (ITK) or 5 h (RLK). The emission ratio (665 nm: 615 nm) was calculated and equation 3 fit to the data to provide a k_{obs} value for each inhibitor concentration using GraphPad Prism version 8.3.1, GraphPad Software, San Diego, California USA. Individual values for k_{inact} and Kᵢ or their ratio k_{inact}/Ki were obtained by fitting equation 4 or 5, respectively to a secondary plot of k_{obs} versus inhibitor concentration (Kitz-Wilson plot).

$$Equations ( 3 ) , ( 4 ) , and ( 5 )$$

$$A = {A}_{∞} + ( {A}_{0} - {A}_{∞} ) {e}^{- {k}_{obs} t} \qquad ( 3 )$$

$${k}_{obs} = \frac{{k}_{inact} [ I ]}{[ I ] + {K}_{i} ( 1 + \frac{[ L ]}{{K}_{d}} )} \qquad ( 4 )$$

$${k}_{obs} = \frac{\frac{{k}_{inact}}{{K}_{i}} [ I ]}{\frac{[ I ]}{{K}_{i}} + ( 1 + \frac{[ L ]}{{K}_{d}} )} \qquad ( 5 )$$

With reference to Equations (3)-(5): A is the tracer-bound enzyme at time t, A₀ is the tracer-bound enzyme at t=0, A_{∞} is the tracer-bound enzyme at t=∞, k_{obs} is the apparent first order rate constant of inactivation, [I] is the concentration of compound, [L] is the concentration of tracer 236, K_{d} is the equilibrium dissociation constant of tracer 236 for the kinase, Kᵢ is the equilibrium dissociation constant of the compound for the inhibitor with kinase for the initial reversible step and k_{inact} is the first order rate constant for the chemical step of inactivation (FIG. 11). Technical triplicates of the progress curves were collected at each determination and the values reported are the mean of three independent determinations.

### Jump Dilution Assay

The assay format is similar to the Competitive Tracer-binding Assay and relies on the reversible binding of tracer 236 to recombinant tagged human ITK. Recombinant human ITK (0.5 μM) in kinase buffer A, was incubated with 1.5 μM CPI-818 or BMS-509744 (MedChemExpress, Monmouth Junction, NJ) for 120 min at room temperature. The mixture was then filtered through a desalting column (Thermo Scientific, Zeba Spin Desalting Column, 40K MWCO) to remove unbound inhibitor. The assay was initiated immediately after the desalting step by diluting the eluate containing the inhibited kinase complex 50-fold into a solution of anti-tag antibody (2 nM final) and excess tracer 236 (40 nM final). Dissociation of inhibitor and binding of tracer was monitored immediately after assay initiation using an EnVision plate reader (excitation 340 nm, dual emission at 665 nm and 615 nm). Reagents including GST-tagged recombinant human ITK, Kinase tracer 236, Lanthascreen Eu-Anti-GST antibody and kinase buffer A were purchased from ThermoFisher Scientific/Invitrogen. Each data point represents the mean of technical triplicates. Equation 3 was fitted to the BMS-509744 data to obtain the rate constant using GraphPad Prism from which the half-life was calculated.

### Glutathione Reactivity Assay

The reactivity of acrylamides with glutathione was determined by measuring the disappearance of starting material and appearance of the adduct with glutathione in HPLC-MS spectra as a function of time. All the stock solutions and solvents were bubbled with nitrogen and all reactions were run under nitrogen at 37° C. A 250 μL volume of 20 mM test compound in DMSO was mixed with 250 μL of 2.0 mM indoprofen (used as internal standard) in DMSO and 4.5 mL of 11.1 mM glutathione in 100 mM potassium phosphate buffer (pH 7.4). Each reaction contained 1 mM test compound, 0.1 mM indoprofen, and 10 mM glutathione in 100 mM potassium phosphate buffer and 10% DMSO. The reaction mixture was stirred at 37° C. for 5 hours and sampled for HPLC-MS analysis every hour. The reaction product, internal standard and starting materials were separated by liquid chromatography using an Agilent Eclipse XBD-C18 column (4.6×150 mm, 3.5 um) and quantified by UV absorption. The mobile phase consisted of solvent A (water) and solvent B (acetonitrile), employing a gradient of 10% to 95% solvent B over 10 min.

### Mass Spectrometry

ITK was obtained as a glutathione S transferase (GST) fusion protein from Cama Bioscience (Product Number 08 081, Lot 13CBS 0356K). The protein was supplied at 586 μg/mL (5.9 μM ITK) in 50 mM TRIS, 150 mM NaCl, 0.05% Brij35, 1 mM dithiothreitol, and 10% glycerol at pH 7.5. The protein construct comprised amino acids 2 to 620 of ITK and is fused to GST at the N terminus of ITK (ITK GST). To prepare the samples for mass spectral analysis, a 2 μM sample of the ITK GST fusion protein was incubated with a 5-fold molar excess of CPI 818 (10 μM). A control, untreated sample of ITK GST was prepared in parallel. The samples were incubated at room temperature for 2.5 hours to allow for complete inhibition of ITK GST by CPI-818. After the incubation phase, samples were frozen and stored at −80° C. Mass spectral analysis was conducted in collaboration with the Stanford University mass spectrometry facility. The control, untreated ITK GST fusion protein, and the CPI 818 inhibited sample were subjected to chymotrypsin digestion and then analyzed by liquid chromatography coupled with tandem mass spectrometry (LC MS/MS). In a typical analysis, peptides were separated using an Acquity M-Class liquid chromatograph (Waters Corporation, Milford, MA) at a flow rate of 450 nl/min. Peptide separation was completed on an in-house pulled and packed fused silica column about 25 cm in length, with an I.D. of 100 microns packed with 1.9 micron C18 stationary phase. An analytical gradient of 80 minutes was used, with mobile phase A being 0.2% formic acid in water, and mobile phase B being 0.2% formic acid in acetonitrile. Ions electrosprayed from this column were detected using an Orbitrap Fusion mass spectrometer (Thermo Scientific, San Jose, CA) operated in a data dependent fashion using both ETD and HCD fragmentation to improve detection efficiency of modified peptides. To analyze these data, raw spectral files were investigated using Byonic v2.10.5 (Protein Metrics, Cupertino, CA) in a targeted database for the protein of interest. Peptides were constrained to 12 ppm mass accuracy for precursor ions, and 0.4 Da mass accuracies for fragment ions detected in the ion trap. Peptides were assumed to be digested in a semi specific fashion, and up to 2 missed cleavages were allowed. Assigned observed peptides were then validated by inspection of the MS 1, MS2, and chromatographic XIC using Byologic (Protein Metrics, Cupertino, CA). This work was supported in part by NIH P30 CA124435 utilizing the Stanford Cancer Institute Proteomics/Mass Spectrometry Shared Resource.

### Autophosphorlyation Assay

HEK293 cells were transiently transfected with a vector encoding full-length human wild type BMX, BTK, ITK, JAK3, TEC, or RLK. Cells were dispensed into 96-well plates at an appropriate cell number previously determined by cell titration. A serial dilution of compound was prepared in 100% DMSO and subsequently diluted in cell culture medium to provide an intermediate solution containing 5% DMSO. The intermediate solution was diluted 10-fold into the cells contained in 96-well plates. The plate included control wells containing no inhibitor (positive control) or a saturating concentration of a reference inhibitor (negative control) at 0.5% DMSO. Cells were incubated with compound at 37° C. for 2 hours before collection and lysis. Cell lysates were transferred to an ELISA plate coated with the appropriate anti-kinase antibody and incubated for 1 h. The lysate was removed, and the wells washed then incubated with an anti-phospho-tyrosine antibody to detect phosphorylated kinase. HRP-conjugated secondary antibody was dispensed to the wells following a wash step and incubated for 1 h at room temperature. The secondary antibody was removed, and the wells washed before addition of HRP substrate followed by incubation at room temperature to allow the reaction to proceed. Absorbance was measured at 450 nm and data were normalized according to equation 1. Inhibition of autophosphorylation was quantified by fitting equation 2 to the log base 10 transformed molar concentration of compound versus the normalized data using GraphPad Prism. Technical duplicates of the concentration response were collected for a single IC₅₀ determination.

pERK and pS6 Flow Cytometry

PBMCs were pelleted (400×g at 4° C. for 5 minutes) and resuspended in RPMI+10% heat-inactivated fetal bovine serum (FBS) at 14.3×106 cells/mL; 70 μL (1×106 cells) were plated in a 96 well U bottom plate and incubated at 37° C. for 1 hour. A 10 mM stock solution of CPI-818 was prepared in DMSO and then serially diluted to 100, 10, 1, and 0.1 μM, which is 10× the final concentration; 10 μL CPI-818 were added to the cells and incubated at 37° C. for 1 hour. CD3-biotin and CD28-biotin were prepared together at 10 μg/mL each, which is 10× the final concentration; 10 μL antibodies were added to the cells and incubated at 37° C. for 15 minutes. Avidin was prepared at 500 μg/mL, which is 10× the final concentration; 10 μL avidin were added to the cells and incubated at 37° C. for 5 minutes to induce TCR crosslinking. A total volume of 100 μL cells were transferred to 200 μL warm 2.4% paraformaldehyde (PFA) in deep well blocks. Pipetting was performed using a liquidator 96 to add reagents to the entire plate with uniform timing. Cells were fixed with PFA at 37° C. for 10 minutes. Cells were pelleted (1000× g for 5 minutes), and the plate was aspirated leaving approximately 100 μL residual volume. The plate was then vigorously vortexed for 10 seconds, and 1 mL −20° C. methanol was added to each well. Cells were stored at 80° C. for approximately 1 month. For antibody staining, cells were pelleted (1000× g for 5 minutes), washed twice with fluorescent-activated cell sorter (FACS) buffer (phosphate-buffered saline [PBS]+1% bovine serum albumin [BSA]+0.1% sodium azide), stained at room temperature for 1 hour, washed twice with FACS buffer, fixed with 1.6% PFA, and acquired on a cytoflex flow cytometer. Inhibition was measured by construction of a concentration response curve of the Log base 10 transformed values of the molar concentration of inhibitor. The IC₅₀ values were obtained by fitting equation 6 to the data.

$$Equation ( 6 )$$

$$Y = A + \frac{A - B}{1 + {10}^{( ( Log ( {IC}_{50} ) - ( I ) ) \times C )}} \qquad ( 6 )$$

With reference to Equation (6): Y is the response, A and B are the top and bottom of the curve, [I] is the compound concentration, IC₅₀ is the compound concentration giving 50% inhibition and C is the slope of the curve.

### Inhibition of PLCγ1 Phosphorylation

Jurkat human acute T cell leukemia cell line (ATCC, TIB-152) was cultured in RPMI 1640 medium supplemented with Penicillin-Streptomycin (Gibco) and 10% fetal bovine serum (FBS) at 37° C. and 5% CO2. 5×10⁷ cells per sample in plain medium were pre-treated with CPI-818 for 1 hour at 37° C., washed and resuspended in 1 mL PBS, and stimulated for 30 seconds with 6 μg biotin anti-CD3 (Invitrogen) plus 1.5 μg avidin (Pierce). The cell pellet was resuspended in NP40 lysis buffer (Invitrogen) containing protease and phosphatase inhibitors cocktail (Cell Signaling Technology), incubated for 30 minutes on ice, and centrifuged at 15, 000×g for 10 minutes. The lysate supernatant from each condition containing 50 μg of total protein was resolved on an 8% Bis-Tris gel (Invitrogen) under reducing condition and transferred onto a nitrocellulose membrane (Invitrogen) using an iBlot2 gel transfer device. Unless specified otherwise, all reagents described above were purchased from Thermo Fisher Scientific. Membranes were blocked for 1 hour at room temperature and incubated overnight at 4° C. with primary antibodies (1:1000 dilution) in Odyssey blocking buffer (LI-COR) with 0.2% Tween 20. Membranes were washed 4 times (5 min each) in Tris-buffered saline, 0.10% Tween 20 and incubated with IRDye 800CW Anti-Rabbit IgG and IRDye 700CW Anti-Mouse IgG (LI-COR) secondary antibodies (1:20,000 dilution) for 1 hour at room temperature. After washes, membranes were imaged in the LI-COR Odyssey® Fc Imaging System. Primary antibodies against PLCγ1, Tyr 783 phosphorylated PLCγ1, ZAP70, Tyr 319 phosphorylated ZAP70, and μ-actin were purchased from Cell Signaling Technology.

### Inhibition of IL-2 Production

Jurkat cells (0.25×10⁶) in Dulbecco's Modified Eagle's medium (Gibco) supplemented with 5% FBS were seeded in each well of a 96 well MultiScreenR HV filter plate (EMD Millipore) placed on top of a 96 well collection plate. CPI 818 was serially diluted in DMSO and added to the culture. After a 2-hour incubation at 37° C. the plates were centrifuged (188×g for 15 seconds) to remove the inhibitor-containing medium from the filter plate. Cells were washed twice by resuspending with 200 μL of medium per well and centrifuged. Cells in each well were then resuspended with 1.25×10⁶ (50 μL) of Dynabeads CD3 (Life Technologies) and incubated for 18 hours at 37° C. Conditioned media was collected by centrifugation (188×g for 1 min). Human IL-2 concentration was measured with the AlphaLISA human IL2 Kit (PerkinElmer) and data collected on an Envision Plate Reader (PerkinElmer). Data were analyzed and graphed using GraphPad Prism version b7.0b. Technical duplicates of the concentration response were collected at each determination.

### T Cell Activation

Fresh PBMCs from healthy human donors were centrifuged and reconstituted at 0.312×106 cells/mL in RPMI 1640 medium+10% heat-inactivated FBS+1× penicillin streptomycin and 100,000 cells/well were distributed into 24 well tissue culture plate. Cells were incubated at 37° C. for 1 hour, followed by the addition of CPI 596 or vehicle. The cells were then incubated at 37° C. for 1 hour, followed by the addition of 1 mg/mL anti CD3 and anti CD28 (both eBioscience). Cells were incubated at 37° C. for 6 days, with supplemental media supplied on day 3. On Day 6, the cells were transferred to a 96 deep well plate, washed, and treated with 50 nM phorbol 12-myristate 13-acetate, 1 μg/mL ionomycin, and 1 μg/mL brefeldin A, and then incubated at 37° C. for 4 hours. The cells were then fixed and permeabilized using eBioscience Fox3/transcription factor staining buffer. Cells were treated with Fc block (BioLegend) and then stained with CD3 BV605, CD45RA PerCP-Cy5.5, Tbet PE, IFNγ AF488, IL-4 APC (BioLegend), CD4 PE-Cy7, cPARP AF700 (BD Biosciences), and CD8 Krome Orange (Beckman Coulter). For flow cytometry, 300,000 cells were acquired on Cytoflex (Beckman Coulter). Data were analyzed using Flowjo software. The percentage of CD4⁺ T cells that were positive for IFNγ and IL 4 were reported.

### ITK Active Site Occupancy

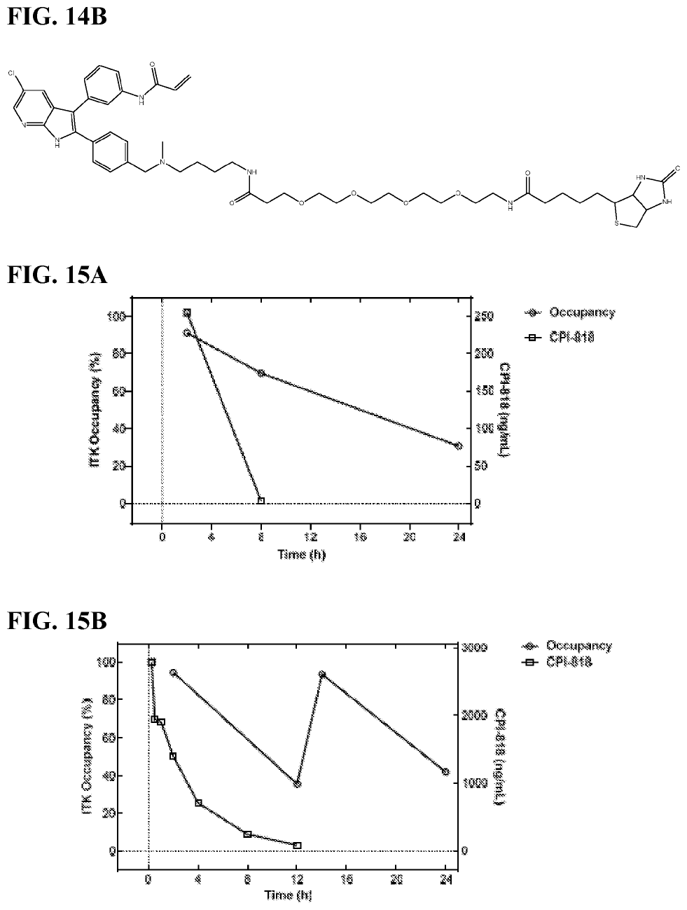

The active site occupancy of ITK by a covalent inhibitor was measured using an irreversibly bound biotinylated probe in an immuno-electrochemiluminescence assay. The binding of an inhibitor and the probe to the ATP binding site is mutually exclusive. In lysates of immune cell lines or immune cells collected from treated animals, the biotinylated probe reacts with free (uninhibited) ITK and is subsequently captured on a streptavidin-coated surface and quantified. The free ITK in each sample is normalized by dividing it by either the lysate protein concentration (BCA protein assay, Pierce) or cell count of the sample. Normalized free ITK values are generated for samples collected prior to and after treatment from which occupancy was calculated using equation 7. Single cell suspensions of Jurkat cells, mouse splenocytes, dog PBMC or purified normal human T cells were prepared. Cell lysis was performed using a buffer containing CelLytic M (Sigma Aldrich) supplemented with protease and phosphatase inhibitors (Pierce). The cell pellet resuspended in lysis buffer was incubated at room temperature for 30 minutes when the lysate was clarified by centrifugation at 20,000×g for 5 minutes at 4° C. A solution of the biotinylated probe CP-613 (27.5 nM final, FIG. 14B) was added to the lysate and incubated with mixing for 60 minutes at room temperature. The lysate mixture (35 μL) was transferred in duplicate to separate wells of a blocked streptavidin-coated plate (Mesoscale Diagnostics) then incubated at room temperature with mixing for 60 minutes. The lysate mixture was removed from the plate and the wells were washed with three rounds of PBS/Tween 20 buffer (Thermo Scientific). Rabbit polyclonal anti-ITK antibody solution (Abcam) was dispensed into the wells of the plate and incubated at room temperature with mixing for 60 minutes. The antibody solution was removed from the wells and washed with three rounds of PBS/Tween 20 buffer. Anti-rabbit antibody (goat) sulfo-TAG (Mesoscale Diagnostics) was dispensed into the wells of the plate then incubated at room temperature with mixing for 60 minutes. The antibody solution was removed from the plate and the wells washed with three rounds of PBS/Tween 20 buffer. Read buffer (Mesoscale Diagnostics) diluted two-fold with water was dispensed into the wells of the plate which was read immediately using an MESO Quickplex SQ 120 instrument.

$$ITK Occupancy ( \% ) = ( 1 - \frac{Posttreatment Free ITK}{Posttreatment Free ITK} ) \times 100 \qquad Equation ( 7 )$$

### Measurement of ITK Turnover

The method utilized the ITK active site occupancy assay to measure either newly synthesized ITK after irreversible inhibition of existing kinase by a covalent inhibitor or persistence of existing enzyme in cells where new protein synthesis was blocked by a sub-cytotoxic concentration of cycloheximide. To measure rates of degradation in Jurkat cells and purified normal human T cells, cycloheximide was added to a culture of 3×10⁶ cells/mL at a final concentration of 50 μM and mixed. The cells were then dispensed into the wells of a 24-well plate (1.0 mL/well) and returned to culture. At timed intervals, the cells from appropriate wells were transferred to a microfuge tube and collected by centrifugation. The cell pellet was stored at −80° C. until the day of the occupancy assay. To measure rates of newly synthesized ITK in cells, existing ITK was first irreversibly inhibited by addition of CP-464 to the culture at a final concentration of either 4, 10, 20 or 50 nM and mixed. The cells were returned to culture for one hour at which time they were collected by centrifugation, washed then resuspended in fresh medium to remove residual CP-464. The cells were then dispensed into the wells of a 24-well plate (3E+06 cells/mL. 1.0 mL/well) and returned to culture. At timed intervals, the cells from appropriate wells were transferred to a microfuge tube and collected by centrifugation. The cell pellet was stored at −80° C. until the day of the occupancy assay. The data were fitted to first order exponential equations for association or decay to obtain the rate constants from which the half-lives were calculated.

### Mouse ITK Occupancy Studies

Male C57Bl/6 mice (6-8 weeks of age) were purchased from Charles River Laboratories. On the day of the experiment 100 μL of 50 mg/kg CPI-818 dissolved in a vehicle composed of 2% Tween 80, 2% Cremophor ELP, 10% PG, 6% PEG 300 and 2% HPC-SL in water or vehicle solution was administered PO. At scheduled time points mice were euthanized by asphyxiation by CO2 and their spleens were collected immediately and placed into ice cold PBS (N=4/timepoint). All animal procedures were conducted in accordance with a protocol approved by the IACUC committee at Corvus Pharmaceuticals.

### Mouse T Cell Adoptive Transfer Colitis Model

To enable long-term dosing in animal studies, a CPI-818-formulated mouse diet (300 mg/kg/day), and a control diet, with macronutrient levels matching the common Teklad 2018 diet were created (Research Diets, Inc.). Adequate drug exposure was confirmed by standard LC-MS method in plasma samples. Female BALB/c mice (11-12 weeks old) and female Fox Chase C.B-17 severe combined immunodeficient (SCID) mice (6-7 weeks old) were purchased from Charles River Laboratories. On study day −7, SCID mice (10/group) began to have ad libitum access to control or CPI-818 diets. A third group on control diet was treated weekly via intraperitoneal (IP) route with 0.5 mg/mouse of anti-IL-12 p40 (clone C17.8) (BioXcell BE0051). On study day 0, SCID mice were injected IP with 4×10⁵ CD4⁺CD45RB^{hi} splenic T cells from BALB/c mice. On study day 48, animals were sacrificed. Colons were harvested for measurements. The distal section was preserved in 10% neutral buffered formalin, embedded into paraffin blocks, and sent to HistoTox Labs, Inc. for histologic analyses. Therapeutic efficacy was based on body weights, colon weights, and colon lengths. This study was carried out at Bolder BioPATH, Inc. in accordance with the test facility standard operating procedures, the World Health Organization Quality Practices in Basic Biomedical Research guidelines, and in compliance with all state and federal regulations.

### Histopathology Analyses

Each slide containing 3 cross sections of the distal colon from each animal was stained with hematoxylin and eosin (H&E), or with an anti-CD3 antibody, and evaluated by a board-certified veterinary pathologist blinded to treatment information. Histologic lesions in H&E-stained slides were scored separately for severity 0-5, where 0=absent, 1=minimal, 2=mild, 3=moderate, 4=marked, 5=severe. Anti-CD3-stained slides were scanned using an Aperio AT2 whole slide scanner. Exclusions were applied to remove debris and tissue artifacts from the analyzed area. An algorithm was applied to the samples using Visiopharm (VIS) image analysis software. A count-based thresholding method was utilized to determine positivity of each pixel on the slide. The average of the three colon samples per slide was taken to determine both the number of immunolabeled CD3 cells and the total area of the colon. The output images underwent a quality control check after analysis was complete. CD3-immunolabeled cell density was calculated per 10,000 μm² using the following formula:

$$CD 3 immunolabeled cells per 10 , 000 μ {m}^{2} = \frac{CD {3}^{+} Cells}{Total Area ( μ {m}^{2} )} \times 10 , 000$$

### Statistical Analyses of In Vivo Studies

The body weights over time between two groups of animals were compared using the two-way analysis of variance (2-way ANOVA) method. Organ weights or lengths were compared using a Student's unpaired, two-tailed, t-test with Welch's correction. All statistical analyses were performed using Prism Version 8.3.0 (GraphPad Software).

### Results

### Design of a Covalent Irreversible ITK Inhibitor

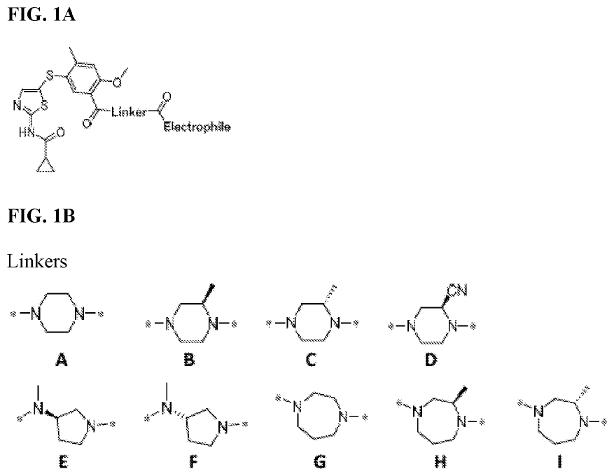

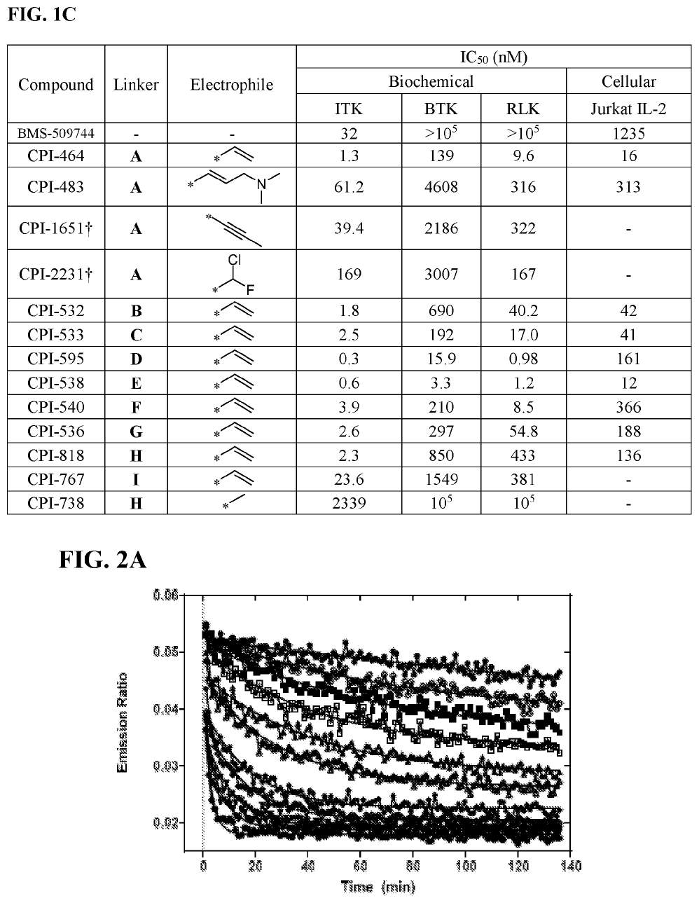

Analysis of ligand bound ITK crystal structures in the Protein Data Bank (PDB) suggested the aminothiazole-based molecular scaffold of BMS-509744 as a suitable starting point for structure-based drug design of ITK-selective covalent inhibitors. BMS-509744 forms two hydrogen bonds with the Met-438 residue in the hinge region and a hydrophobic interaction with the gatekeeper residue Phe-435 (PDB entry 3MJ2). Kutach et al, Chem Biol Drug Des 76, 154-163 (2010). The piperazine-acetamide fragment projects toward Cys-442, providing a useful vector for ligand modification with different electrophilic substituents to engage Cys-442 in covalent bond formation. Guided by modeling efforts we first designed CP-464 by adding an acrylamide capable of interacting with Cys-442. In addition, we replaced the benzylamine moiety of BMS-509744 with the less bulky and less polar cyclopropyl-substituent to achieve better overall drug-like properties. Docking of CP-464 into the ITK active site suggested retention of key ligand-protein interactions that were observed for BMS-509744. The distance between the terminal carbon atom of the acrylamide group and the sulfur atom of Cys-442 was 4.1 Å, making it possible to form a covalent bond through a Michael addition reaction. CP-464 demonstrated improved ITK potency compared to non-covalent inhibition by BMS-509744 (FIG. 1) as well as improved functional activity measured by inhibition of IL-2 production by Jurkat cells. However, CP-464 displayed a modest 7-fold biochemical selectivity over RLK and optimization was continued addressing diamine linkers and electrophilic groups. Compounds with unsubstituted acrylamide moieties demonstrated the highest potencies, while activities of amine-substituted acrylamides (CP-483), propargylamides (CP-1651) and chlorofluoroacetamides (CP-2231) were comparable to, or less potent than the reversible inhibitor BMS-509744. Compounds with piperazine- and amino-pyrrolidine-based linkers showed moderate to no selectivity over BTK and/or RLK kinases. Some improvement in selectivity was achieved for the homopiperazine-containing analog CP-536. Introduction of a chiral methyl into the 7-membered homopiperazine ring allowed further improvement of selectivity and yielded the potent and highly selective inhibitor CPI-818. Presumably, the chiral methyl acts as an anchor to stabilize one of the conformations of the 7-membered ring that directs the acrylamide fragment towards Cys-442 facilitating irreversible covalent bond formation between CPI-818 and ITK via Michael reaction.

### Kinetics of Covalent Inhibition by CPI-818

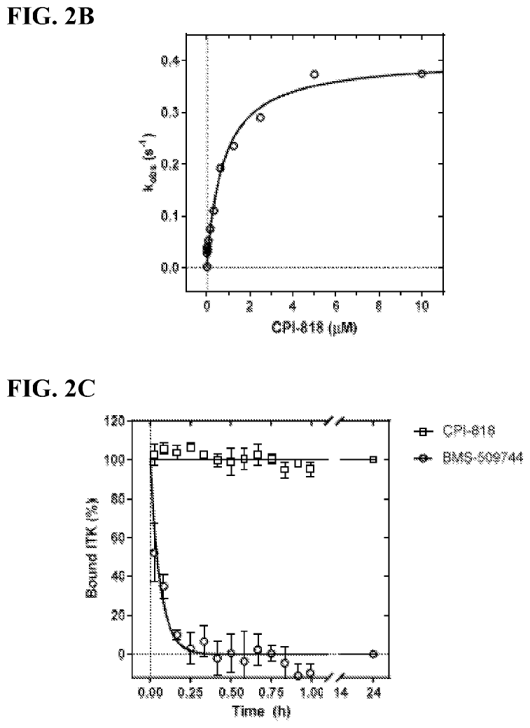

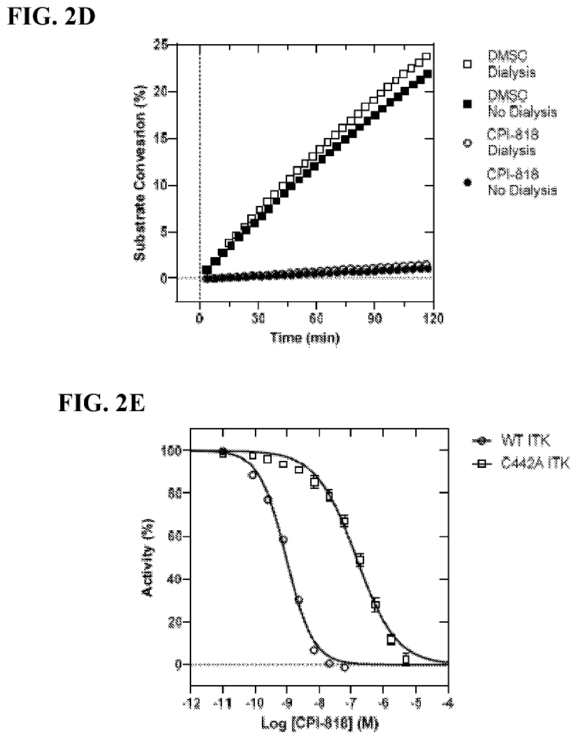

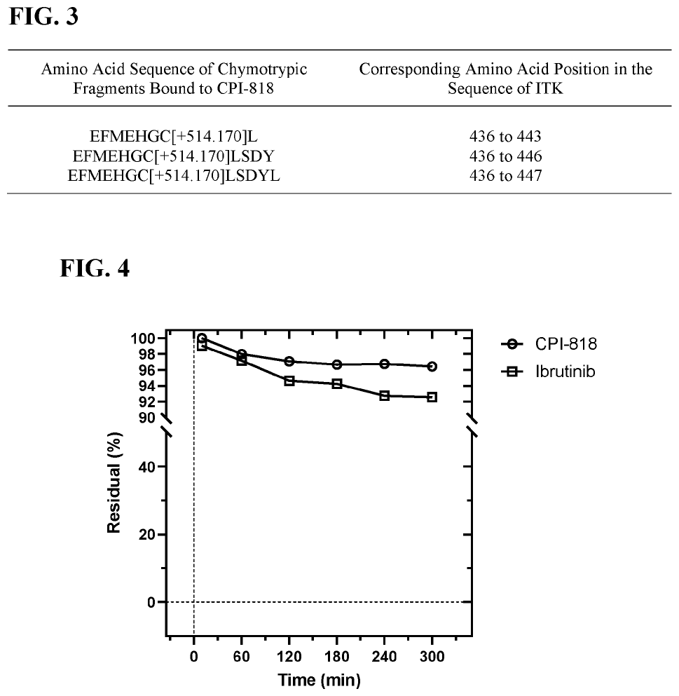

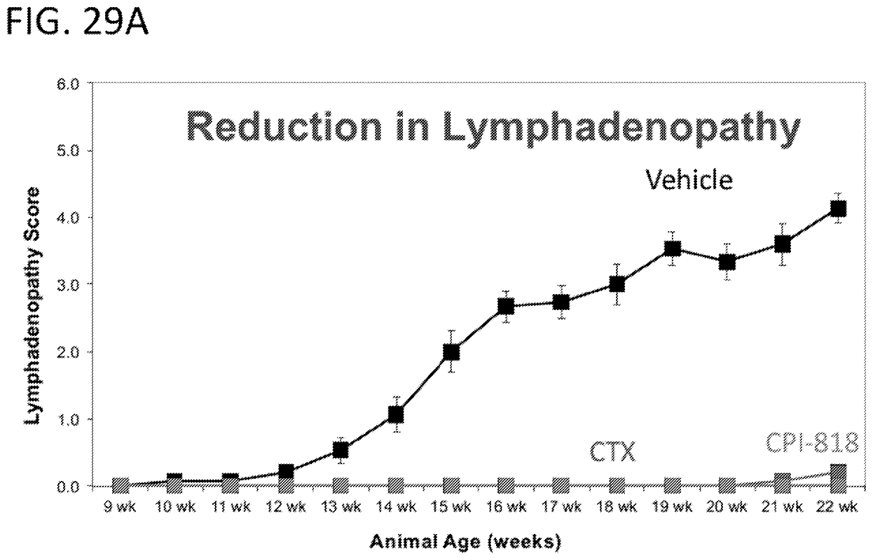

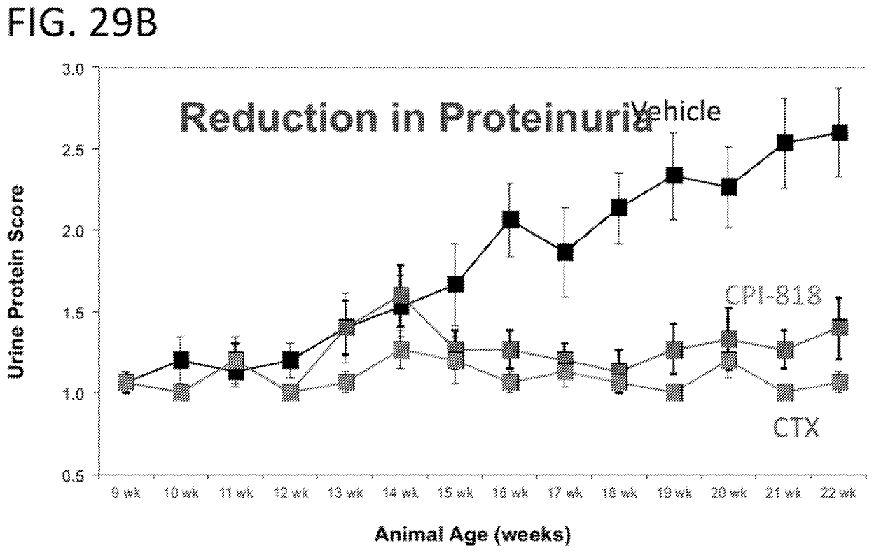

Irreversible and covalent inhibition of ITK by CPI-818 was demonstrated using complimentary biochemical and biophysical techniques. Irreversible inhibition is time-dependent and the reaction proceeds toward completion rather than equilibrium (FIG. 11). Strelow, SLAS Discov 22, 3-20 (2017). To demonstrate time-dependent inhibition, CPI-818 was evaluated in a competitive tracer binding assay performed in a continuous mode to monitor the fraction of ITK active sites occupied by the inhibitor. Progress curves of tracer binding to ITK in a mixture of the kinase and CPI-818 displayed decreasing binding over the assay time course indicating a time-dependent reduction in free ITK active sites available to the tracer as the sites became occupied by CPI-818 (FIG. 2A). Extending the use of this assay by collecting progress curves of probe binding for a series of compound concentrations permits analysis of the inactivation kinetics to reveal the contributions of molecular binding affinity and chemical reactivity to overall potency. Fitting equation 1 to the progress curves provided pseudo-first-order rate constants for inactivation (k_{obs}) that increased with increasing concentrations of CPI-818. A Kitz-Wilson plot of k_{obs} gave a hyperbola typical of an enzyme inactivator with a mechanism conforming to scheme 1 in FIG. 11. Fitting equation 2 to this data gave k_{inact}/Kᵢ=1.37×10⁻² μ⁻¹s⁻¹ for CPI-818 inactivation of ITK (FIG. 2B). In addition, we investigated the reversibility of inhibition by CPI-818 and the non-covalent inhibitor BMS-509744 using a jump-dilution assay format where preformed enzyme-inhibitor complex was diluted into a solution containing an excess of competitive tracer. Dilution of the BMS-509744-ITK complex into a solution of the tracer resulted in rapid dissociation of the inhibitor and binding of the tracer to the enzyme's unoccupied active site. In contrast, the CPI-818-ITK complex did not dissociate upon dilution and blocked binding of the tracer, consistent with CPI-818's irreversible mode of inhibition (FIG. 2C). The BMS-509744 complex displayed a half-life of 2.6 minutes under these conditions while blockade of tracer binding to ITK by CPI-818 persisted for greater than 24 h. Similarly, kinase activity was not recovered for CPI-818 inhibited ITK following extensive dialysis of the inhibited complex and dilution into substrate, indicating irreversible inhibition by CPI-818 (FIG. 29). In addition, an analog of CPI-818 (CP-738) was synthesized in which the acrylamide was replaced with an acetamide. The acetamide lacks a reactive electrophile to participate in covalent bond formation with Cys-442 and CP-738 consequently displays much weaker reversible inhibition of ITK (IC₅₀=2.3 μM, FIG. 1). In a complimentary approach, substitution of the cysteine with alanine at position 442 of ITK removes the nucleophilic cysteine residue on the enzyme preventing the covalent interaction between ITK and the acrylamide of CPI-818. Loss of the ability to form a covalent bond with the mutated kinase decreased the IC₅₀ of CPI-818 at Cys442Ala ITK by >100-fold compared to the wild-type enzyme when measured under similar conditions (FIG. 2E). Finally, a chymotrypic digest of CPI-818-inhibited ITK was analyzed by LC MS/MS to detect the presence of the covalently linked inhibitor to the enzyme. The digest generated 247 peptide fragments and their sequences were identified through deconvolution of the mass spectra. A set of three nested peptides were detected in the sample treated with CPI-818 that were absent from the untreated control sample (FIG. 3). Each of the three peptides contained Cys-442 and their masses were shifted by the mass of CPI-818 when compared to the corresponding unmodified peptide sequence. The presence of the three unique peptide fragments generated by CPI-818 treatment is consistent with the inhibitor covalently labeling Cys-442. There were no other peptide fragments detected that were modified by CPI-818. Collectively these data demonstrate that CPI-818 is an irreversible and covalent inhibitor of ITK.

### Kinase Selectivity of CPI-818

The five members of the TEC kinase family and six other enzymes of the human kinome contain a cysteine in a position homologous to Cys-442 in ITK and could potentially be irreversibly inhibited by CPI-818 (13). Liu et al, Chem Biol 20, 146-159 (2013). To investigate the selectivity against the 11 cysteine-containing kinases, IC₅₀ values for CPI-818 at each enzyme were obtained. CPI-818 displayed an IC₅₀=6.5 nM for ITK and was at least 80-fold selective over the remaining cysteine-containing kinases (FIG. 12). CPI-818 was then profiled against the human kinome at the single concentration of 1.0 μM. CPI-818 displayed a high degree of selectivity where only eight kinases were inhibited ≥65% and only the targeted kinase, ITK, was inhibited by >95%. Importantly, other kinases in the T cell receptor pathway were not inhibited by CPI-818 (kinase, percent control: Lck, 89; Fyn, 99; ZAP-70, 59; RLK, 70). Therefore, the functional effects caused by the molecule following TCR engagement can be attributed to ITK inhibition and not to another kinase in the signaling pathway. An important goal of our chemical design strategy was to achieve selective inhibition of ITK while sparing RLK to avoid the potentially unfavorable effects on T-dependent immunity. To quantify the degree of selectivity we measured k_{inact}/Kᵢ values at the two kinases and found CPI-818 is 115-fold selective toward ITK over RLK. Inspection of the Kitz-Wilson plot for ITK revealed k_{obs} was saturable and estimates for constants governing both the reversible and irreversible steps of inactivation could be obtained (FIG. 2B). After correction for the concentration of competing tracer, we measured Kᵢ=520±110 nM for the reversible step and k_{inact}=0.0067±0.0004 s⁻¹ for the chemical step. The Kᵢ value for the initial reversible interaction is consistent with the reversible inhibition displayed by CPI-818 at Cys442Ala ITK (Kᵢ=410±140 nM). In contrast to ITK, k_{obs} for RLK inhibition by CPI-818 was not saturable under similar conditions and only the k_{inact}/Kᵢ value could be fitted to the data where a value of 1.13×10⁻⁴ μM⁻¹ s⁻¹ was obtained. This non-saturable behavior over similar inhibitor concentrations indicates a much higher Kᵢ value for the initial reversible step of RLK inhibition where the affinity is expected to be comparable to that displayed by CP-738 at RLK (Kᵢ=2.3±0.2 μM). Potent and selective inhibition of ITK was maintained in the cellular context where CPI-818 competes with cytoplasmic concentrations of ATP. To evaluate selectivity in a functional assay against JAK3 and the TEC family kinases we employed an autophosphorylation assay where each full-length kinase was transiently and separately expressed in HEK293 cells. In this assay for inhibition of ITK autophosphorylation, the molecule had an IC₅₀=45 nM and was 40-fold selective over TEC kinase and >80-fold over the other four members of the TEC family and JAK3 kinases (FIG. 12). It is conceivable that the expression levels of the kinases ectopically expressed in HEK293 cells differ and the compound may appear more potent toward a kinase with low abundance. However, levels of autophosphorylated kinases in the uninhibited control samples were no more than 3-fold different across the kinases evaluated and were not significantly different between ITK and RLK (1.4-fold).

An often-cited concern for drugs containing an acrylamide is their potential for non-specific reactivity with thiols and other nucleophilic groups. We assessed the non-specific reactivity of CPI-818 at 37° C. using physiological levels of glutathione as a surrogate nucleophile and compared it with Ibrutinib. CPI-818 was stable under these conditions with only 4% loss after 5 h, compared to the 7.5% loss over the same period suffered by the approved acylamide-containing drug Ibrutinib (FIG. 4).

### CPI-818 Inhibits TCR Signaling Downstream of ITK and Blocks IL-2 Production

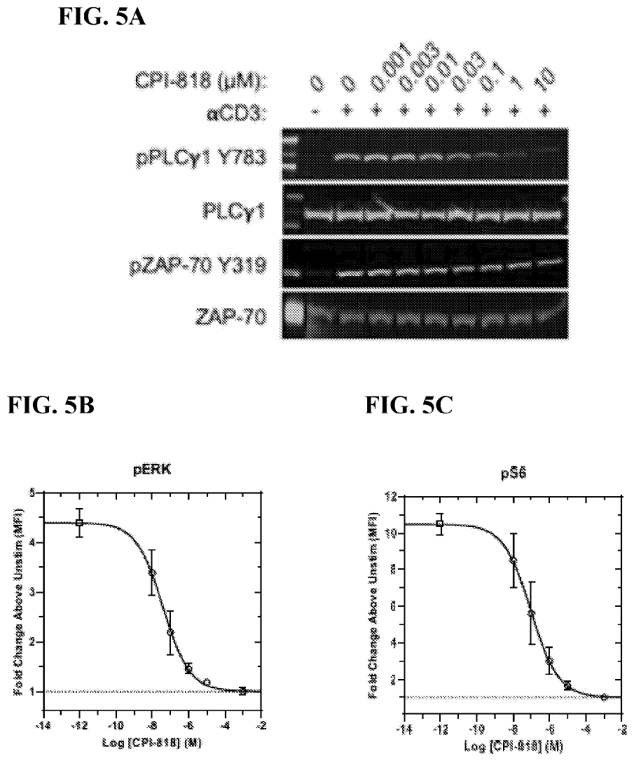

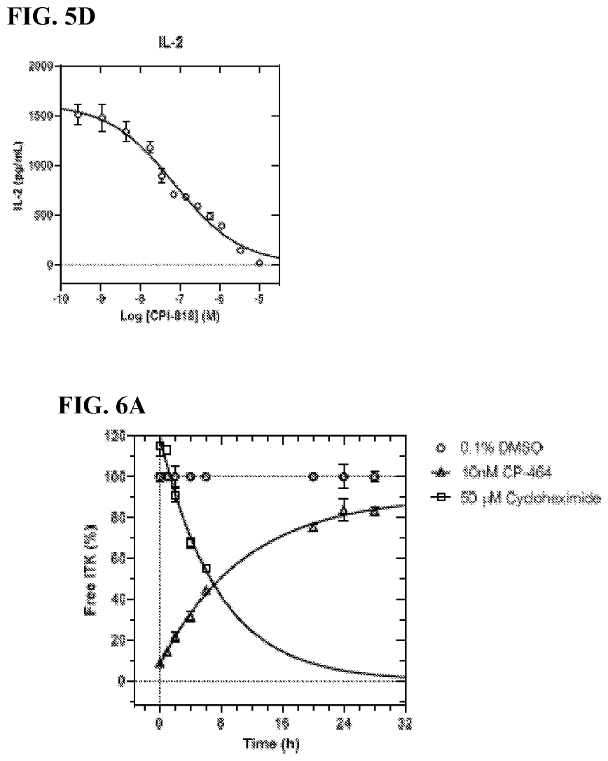

We next investigated the effect of CPI-818 on TCR signaling. Upon TCR engagement, receptor-associated Lck phosphorylates ZAP-70 and ITK. Activated ITK relays the signal through autophosphorylation and phosphorylation of tyrosine 783 on PLCγ1. In the Jurkat T cell line stimulated with anti-CD3, CPI-818 dose-dependently inhibited the phosphorylation of PLCγ1, but not ZAP-70 (FIG. 5A). The effects on the downstream signaling molecules ERK and ribosomal protein S6 were evaluated in primary CD4+ T cells from healthy donors. CPI-818 dose-dependently inhibited anti-CD3/CD28 stimulated phosphorylation of ERK and S6. Under these conditions phospho-ERK and phospho-S6 gave IC₅₀ values of 39 nM and 96 nM, respectively (FIGS. 5B-5D). The secretion of IL-2 is a critical and early landmark of T cell activation and is often used to measure the functional impact of TCR signal transduction inhibitors. To assess the potency of covalent inhibition of ITK by CPI-818, Jurkat cells incubated with CPI-818 were washed extensively and stimulated with anti-CD3 for 18 hours. CPI-818 dose-dependently inhibited IL-2 secretion with a mean IC₅₀ of 136 nM (FIGS. 5B-5D).

### Determination of ITK Turnover Rate

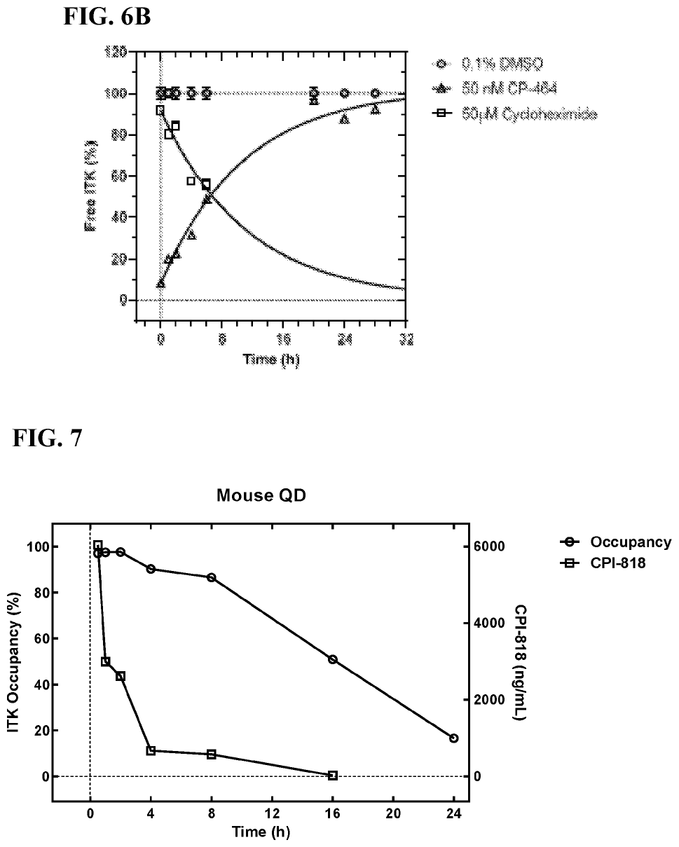

Since CPI-818 does not dissociate from ITK, relief of inhibition and restoration of ITK kinase-dependent pathway activity occurs only after the inhibitor has been cleared and new ITK synthesized. To investigate the durability of irreversible ITK inhibition, the turnover of ITK in Jurkat cells and primary human T cells was measured using the ITK occupancy assay. To quantify the uninhibited form of ITK we developed a chemical probe-based method using electrochemiluminescence to measure the amount of probe-labelled ITK. The probe is a biotinylated covalent ITK inhibitor that reacts with unoccupied ITK and captures it on a streptavidin coated surface. Detection of captured ITK is subsequently achieved by conventional ELISA methodology. Using this approach, we demonstrated titration of ITK by the tool compound CP-464 in Jurkat cells where the IC₉₀=15 nM and exposure to higher concentrations of the compound resulted in complete target engagement under the assay conditions (FIG. 14). We next investigated degradation of ITK in Jurkat cells using the protein synthesis inhibitor cycloheximide to block new ITK synthesis. Cycloheximide (50 μM) did not affect cell viability measured by cellular ATP levels during the initial 6 hours of culture. Analysis of decreasing levels of ITK detected over the first 6 hours using the occupancy assay gave T_{1/2}=6.9±1.3 h for ITK degradation in Jurkat cells (FIG. 6A). To measure the synthesis of new kinase we first blocked the active site of existing ITK using a range of CP-464 concentrations. The covalent inhibitor was subsequently removed by extensive washing before returning the cells to culture. Increases above baseline ITK accessible to the probe after removal of inhibitor were attributed to newly synthesized enzyme. As expected, the initial amount of uninhibited ITK in the culture following incubation and washout was dose-dependent on CP-464, and the rate of new enzyme synthesis did not vary between these conditions. Data fitting of newly synthesized ITK gave a T_{1/2}=8.2=1.2 h in Jurkat cells (FIG. 6A). The rates for degradation and synthesis are similar in these cells and indicate a steady state level of enzyme is maintained in their resting state. Using the same approach, we measured ITK turnover in purified primary T cells from normal donors and obtained half-life values of 6.6±0.6 h and 7.6±3.0 h for degradation and synthesis, respectively (FIG. 6B). Again, the rates of the opposing processes suggest turnover of the enzyme is tuned to maintain a constant level of ITK in resting cells and that the kinase in the acute T cell leukemia cell line and primary human T cells are turned over at comparable rates. These data also suggest a covalent inhibitor that is cleared rapidly from the circulation may need to be dosed more frequently than once per day to maintain high levels of target engagement as newly synthesized enzyme replaces the inhibitor-inactivated pool.

Ex Vivo ITK Occupancy in Mice Dosed with CPI-818

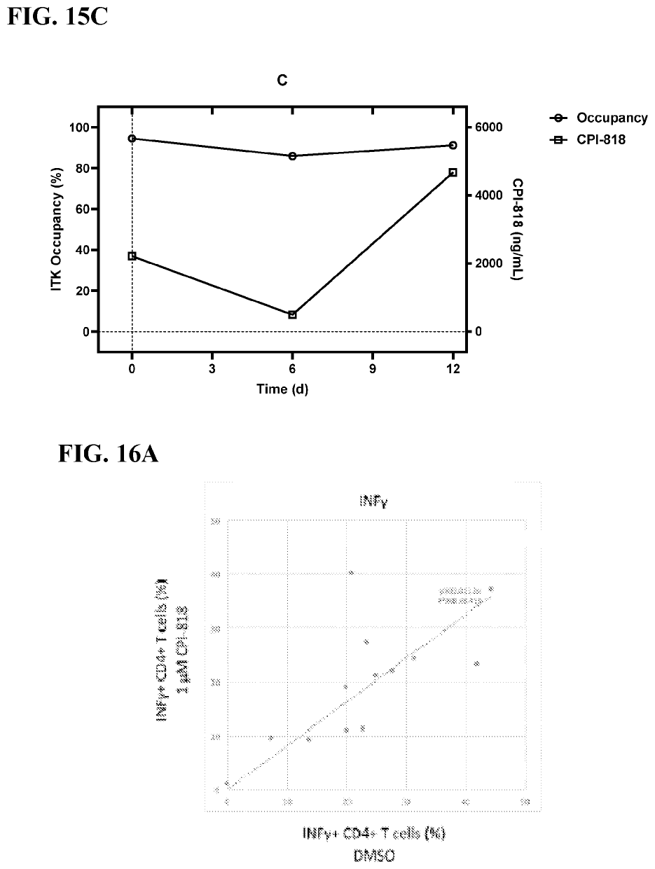

We next investigated the pharmacokinetic and pharmacodynamic properties of CPI-818 after oral (PO) administration to mice. Plasma and splenocytes were harvested at intervals over 24 h following a single 50 mg/kg PO dose. CPI-818 was rapidly absorbed (Tₘₐₓ=30 min) and cleared from circulation with a half-life of 2.4 h. The molecule displayed very high levels of ITK occupancy that were maintained between 87% to 98% in splenocytes during the first 8 h while over the same period its plasma concentration had decreased 10-fold (FIG. 7). Occupancy decreased to 17% at 24 h in the absence of detectable CPI-818 in plasma. The PK/PD relationship of CPI-818 was further explored following a single dose in dogs and BID dosing in mice (FIGS. 15A-15B). In the dog study, occupancy measured in PBMC at 2 h after administration was 91% and decreased to 31% at 24 h when the compound was not detected in plasma. BID dosing in mice led to 95% occupancy in splenocytes 2 h after dosing and decreased to 36% just prior to the second dose at 12 h. Plasma compound levels demonstrated the molecule was essentially cleared from circulation during the 12 h dosing period. The repeat dose provided the same range of occupancies as the first dose measured at 2 h and 12 h following administration. The high levels of ITK occupancy that persisted beyond the compound's clearance from plasma observed in these studies is consistent with the irreversible binding mode of CPI-818 where recovery of unbound ITK is dependent on de novo synthesis of the targeted kinase in the absence of the drug. A mouse chow formulation was then developed to deliver 300 mg/kg/day in diets to facilitate evaluation of disease progression in response to ITK inhibition in model animals. This approach also provided very high levels enzyme occupancy, ranging between 86% to 95%, with continuous target coverage and a facile route of administration (FIG. 15C).

### Effect of Selective ITK Inhibition by CPI-818 on Th Cell Differentiation

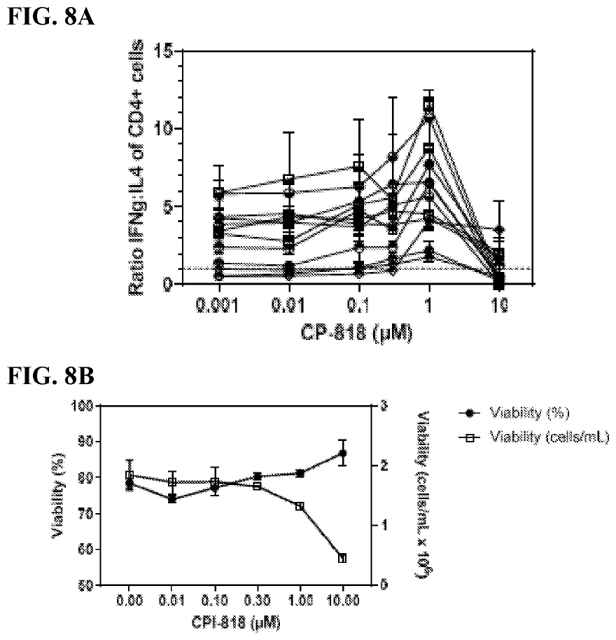

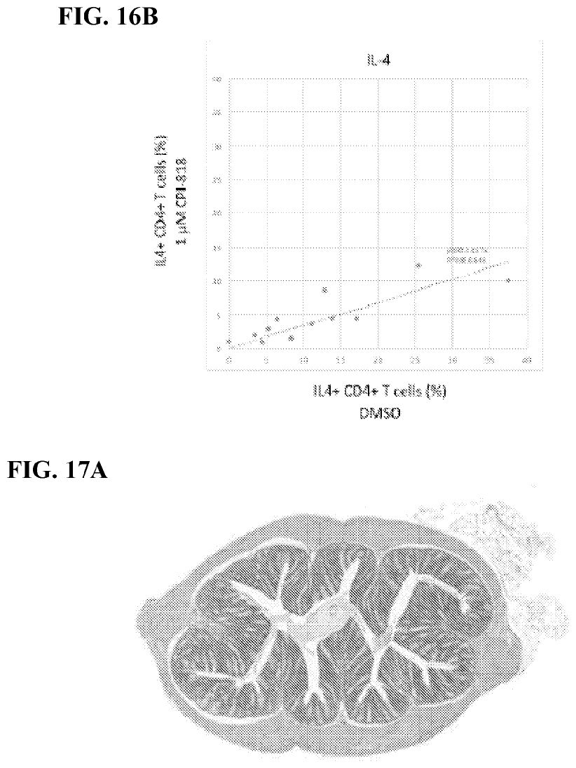

Immunophenotyping of lymphocyte subsets in ITK-null mice suggests highly selective inhibition of ITK, while sparing RLK, can skew T-helper cells toward the Th-1 phenotype. We therefore assessed the effect of CPI-818 on differentiation and activation of helper T cell subtypes. Levels of intracellular cytokines for Th1 (IFNγ) and Th2 (IL-4) were quantified in PBMCs activated with TCR-crosslinking and cultured in the presence of CPI-818. There was very little effect (average <20%) on the percentage of IFNγ⁺CD4⁺ T cells at CPI-818 concentrations up to 1 μM, but at 10 μM IFNγ-positivity was robustly inhibited. IL-4 production was more sensitive to CPI-818 at both 0.3 and 1 μM, with complete inhibition observed at 10 μM. Treatment with 1 μM CPI-818 had a small but significant effect on IFNγ production (average 19% across samples). In contrast, 1 μM CPI-818 strongly inhibited the percentage of CD4⁺ T cells that produced IL-4 (average 64%) (FIGS. 16A-16B). Therefore, 1 μM CPI-818 induced Th1-skewing, with a significant increase in the ratio of IFNγ⁺CD4⁺ T cells to IL-4⁺CD4⁺ T cells (p<0.001) (FIG. 8A). Viability assessment revealed that high concentrations of CPI-818 reduced the total number of cells while maintaining cell viability (FIG. 8B), consistent with a mechanism where 10 μM CPI-818 prevents T cell activation and proliferation without inducing direct T cell killing. Therefore, CPI-818 preferentially inhibits T cell production of the Th2 cytokine IL-4 compared to IFNγ at lower concentrations, and abrogates total T cell proliferation at 10 μM.

### Selective Inhibition of ITK Preserves NK-Mediated ADCC

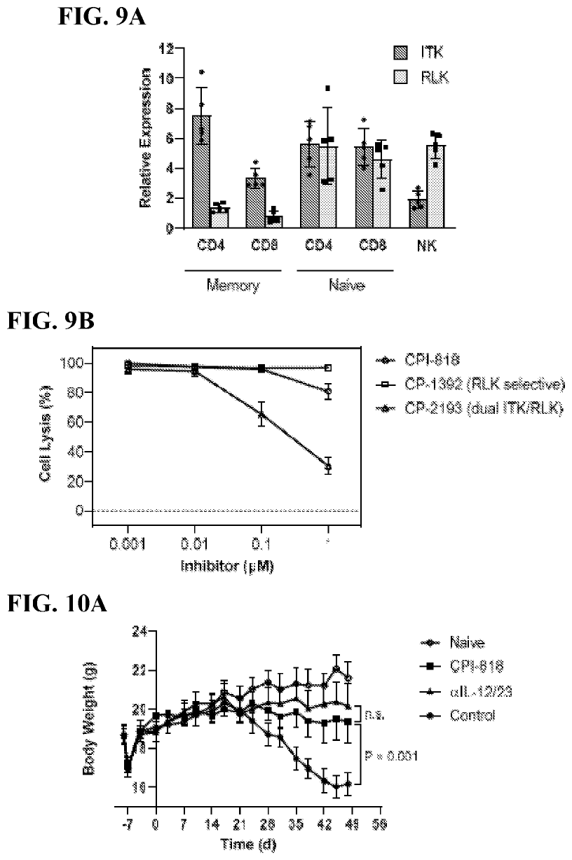

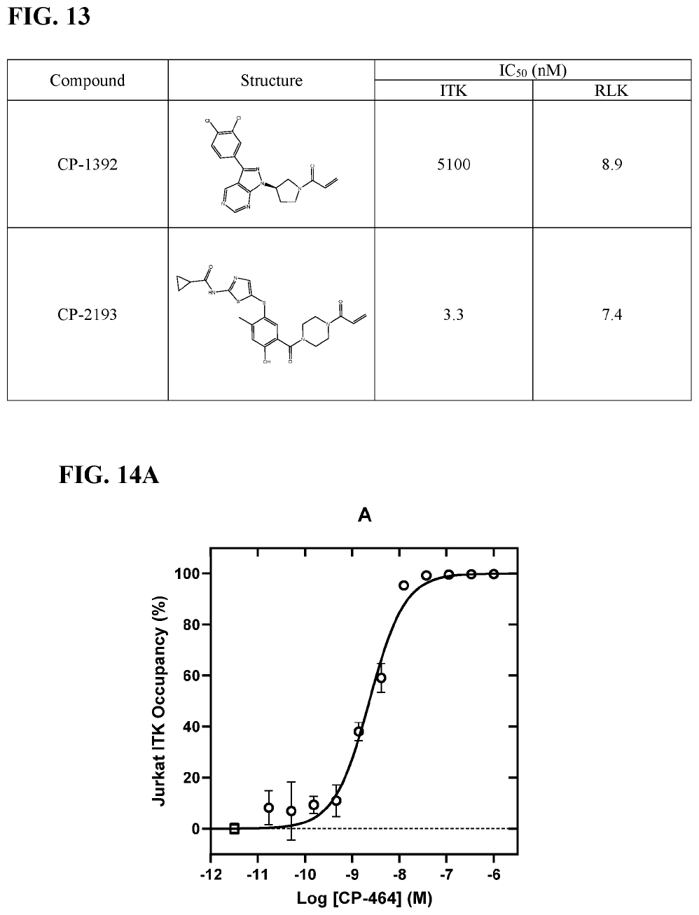

NK cells express both ITK and RLK (FIG. 9A). ITK has been shown to be involved in FcγRIII signaling and antibody-dependent cellular cytotoxicity (ADCC) and RLK may play a similar role. We evaluated the effect of selective ITK inhibition by CPI-818 on NK-mediated ADCC and compared it to an RLK-specific inhibitor and to a dual selective compound (FIG. 13). Peripheral blood NK cells from 7 healthy donors were co-cultured with an anti-CD20 bound target B-cell line at a ratio of 10:1 for 18 hours in the presence of the inhibitors over a range of concentrations up to 1 μM. Cell lysis was detected by flow cytometry using viability dye staining. As expected, the dual ITK/RLK inhibitor CP-2193 showed dose-dependent inhibition of ADCC. In contrast, ITK selective inhibition by CPI-818 decreased ADCC by 20% at 1 μM while the RLK-specific compound CP-1392 showed no decrease in ADCC (FIG. 9B). These data show NK cell ADDC function is maintained following ITK inhibition alone, but not following ITK/RLK dual inhibition, which is consistent with a redundant role for these two kinases in NK cells.

### Orally Administered CPI-818 is Efficacious in a Murine Model of Colitis

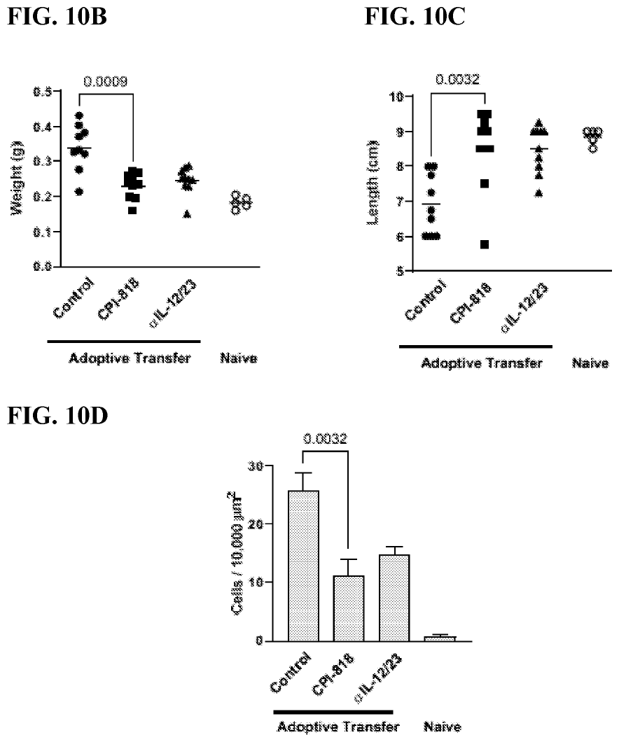

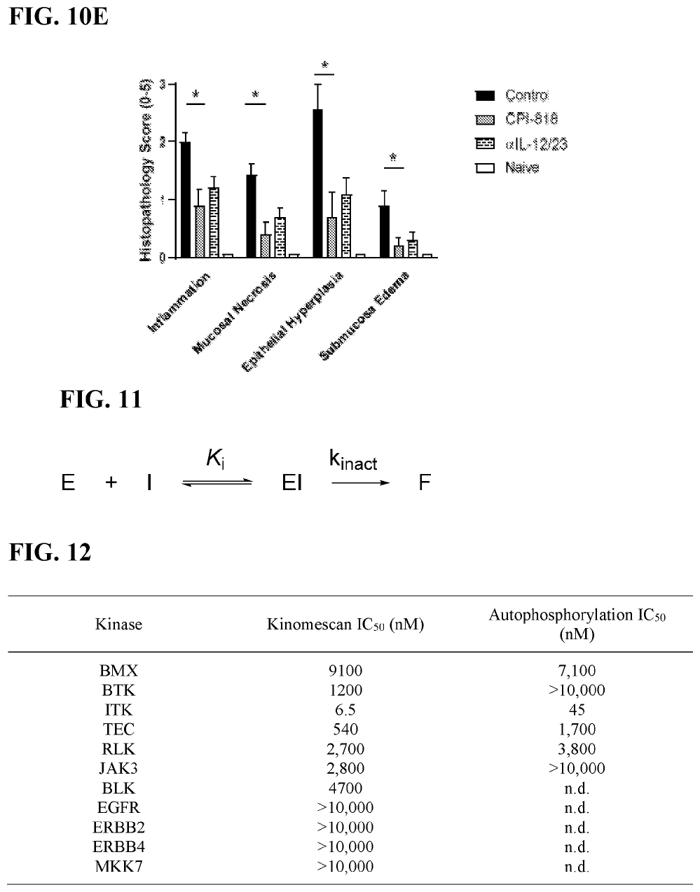

We have demonstrated that CPI-818 selectively inhibits ITK, and blocks TCR-induced cytokine (IL-2) production in T cells. In addition, CPI-818 is orally bioavailable and exhibits sustained target occupancy in vivo. These data suggest the potential of this compound as a therapeutic for diseases caused by autoreactive T cells, such as ulcerative colitis. In a widely used model of colitis (Koboziev et al, Inflamm Bowel Dis 17, 1229-1245 (2011); Leach et al, Am J Pathol 148, 1503-1515 (1996)), CD4⁺CD45RB^{hi} T cells were transferred from BALB/c mice to immunodeficient C.B17 SCID mice. Beginning at 2 weeks post-transfer animals in the vehicle-treated group suffered from diarrhea and progressive weight loss. By contrast, SCID mice that began treatment with CPI-818 one week prior to the transfer were protected from colitis to a similar degree as mice treated with anti-IL-12/IL-23, a positive control therapeutic in this model (Dann et al, J Immunol 201, 548-559 (2018). (FIG. 10A). Consistent with these observations, necropsy at 7 weeks post-transfer indicated that CPI-818-treated mice maintained colon weights and lengths within the ranges of naïve animals that had not received T cell transfer (FIGS. 10B-10C).

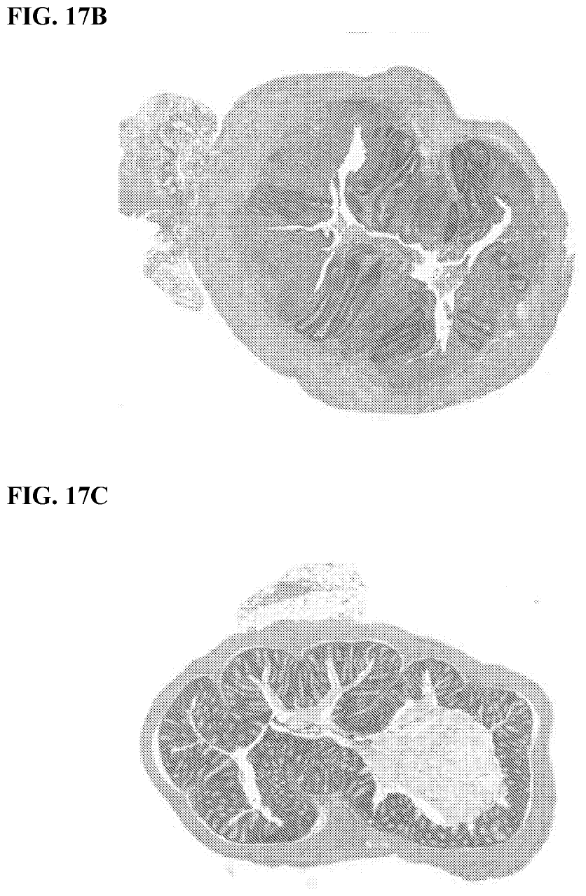

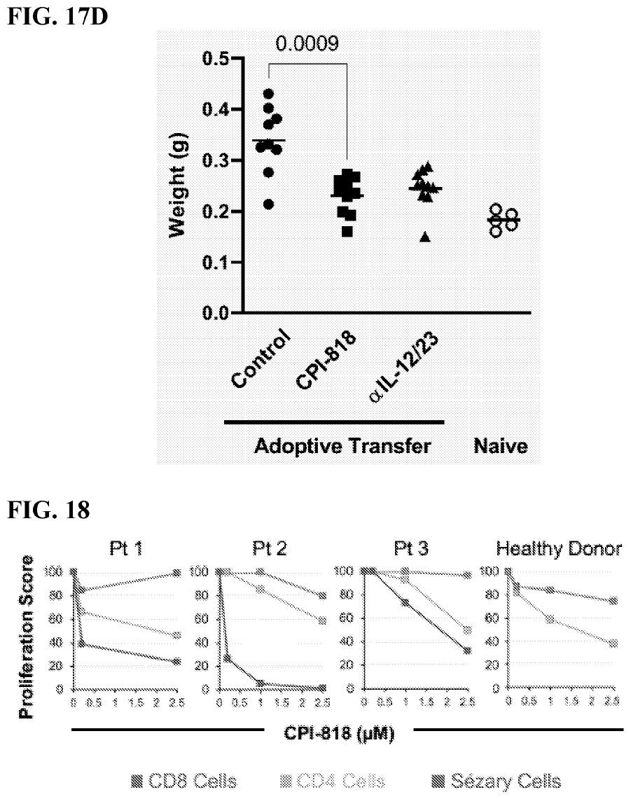

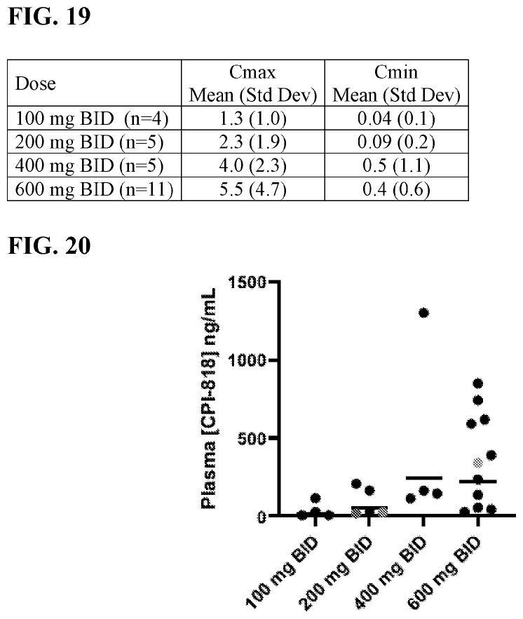

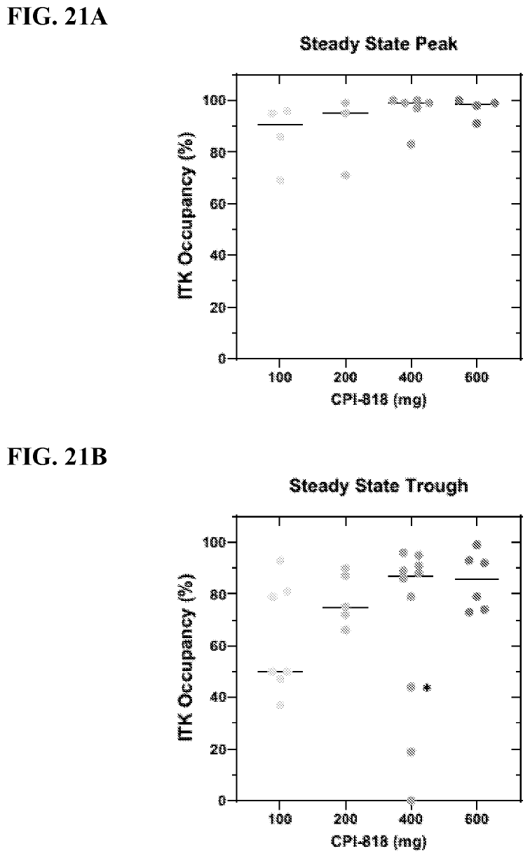

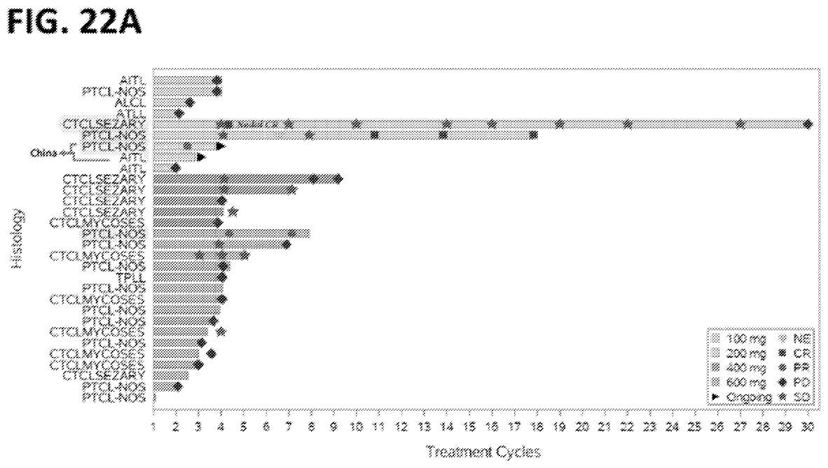

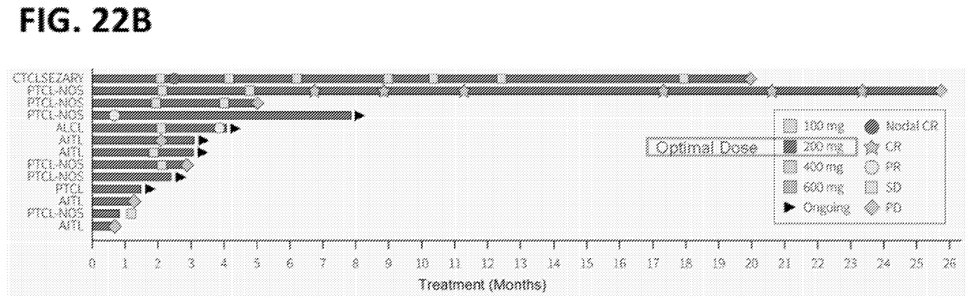

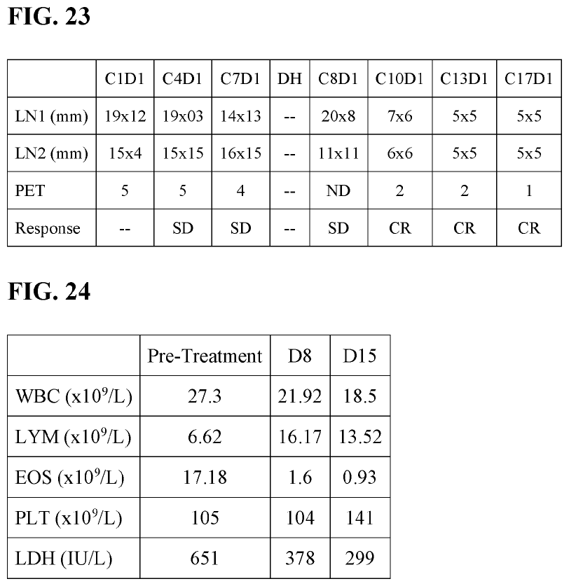

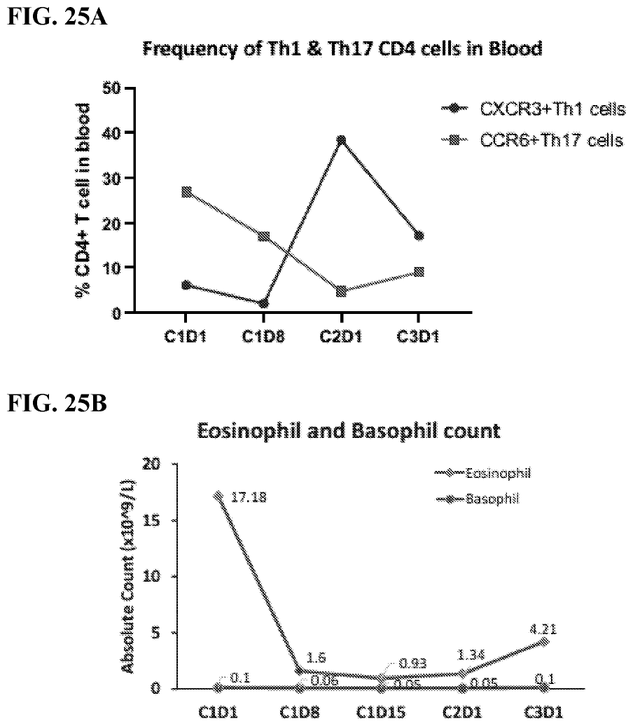

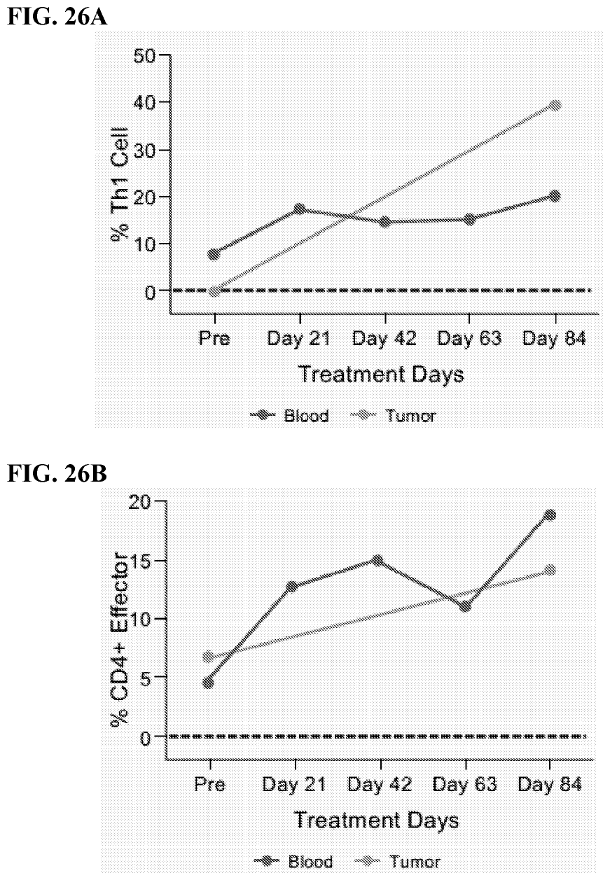

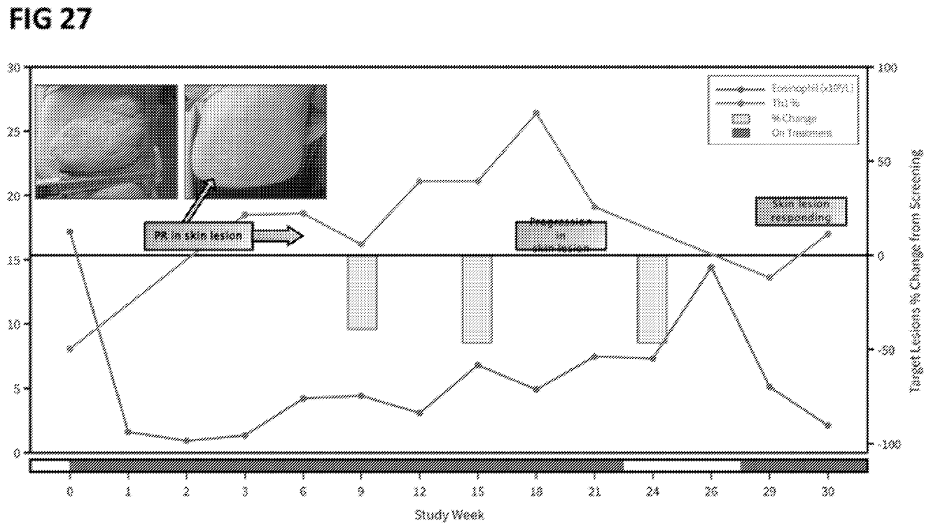

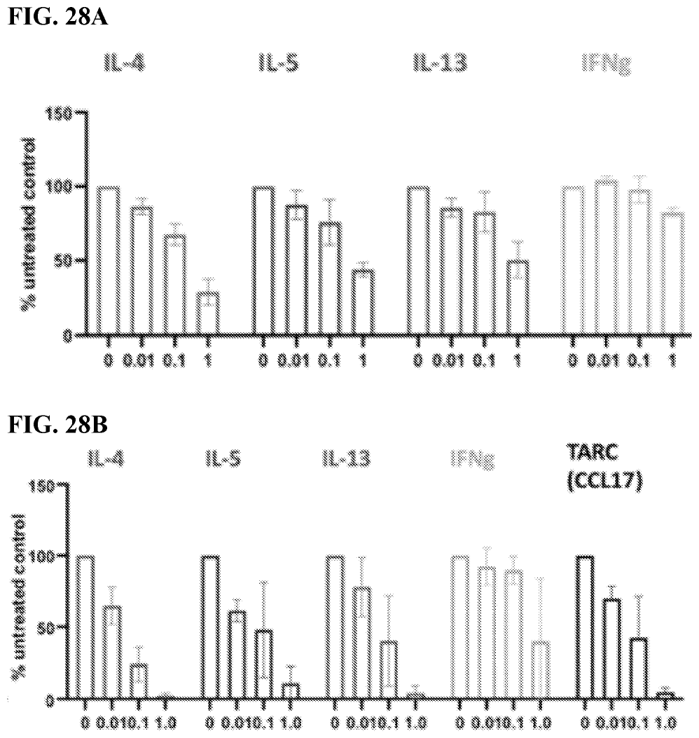

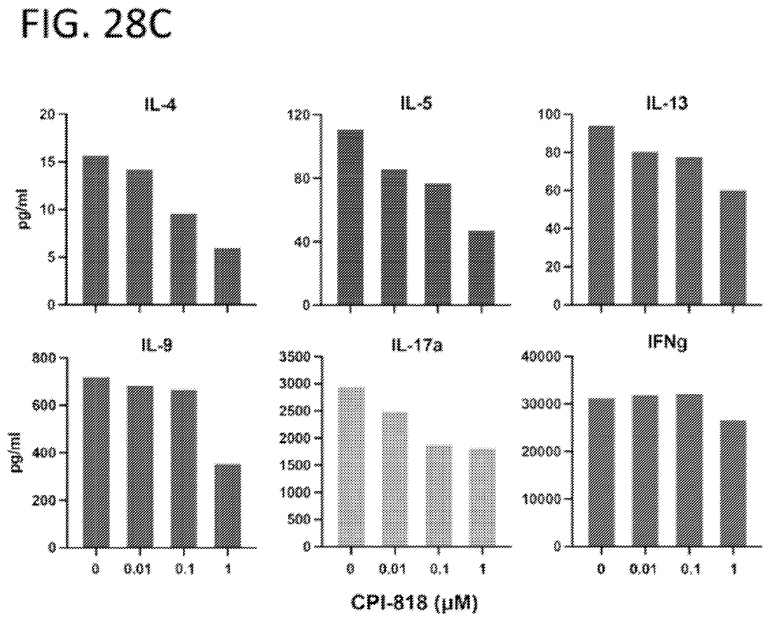

Histological analysis of colon sections indicated that CPI-818 blocked the infiltration of CD3⁺ T cells (FIG. 10D). In addition, the severity of inflammation, mucosal necrosis, epithelial hyperplasia, and submucosa edema among samples were compared. Colons from the vehicle group showed significant inflammation of the mucosa and submucosa layers with infiltration of lymphocytes, neutrophils and macrophages. Mucosal necrosis in these samples was characterized by crypt distortion and loss of colonic glands. Other lesions included epithelial hyperplasia and submucosal edema, which is the expansion of fluid-filled spaces and distention of lymphatic vessels in the tissue. Treatment with CPI-818 or anti-IL-12/23 significantly reduced the severity of these lesions (FIGS. 10E, 17A-17C).

### Discussion

Dysregulated activation and expansion of T cells underlies many human autoimmune and inflammatory diseases. Agents that reduce aberrant TCR signaling without strongly inhibiting overall T cell immunity would be of significant clinical benefit. To this end, ITK is an attractive target due to its selective expression in T and NK cells, and genetic observations that ITK knockouts, or models where ITK is replaced with a kinase-dead allele, together demonstrate that ITK modulates T cell activation and effector functions while allowing for T-dependent immunity. This modulatory role for ITK is thought to be due to partial redundancy between ITK and the structurally similar T cell kinase, RLK. For this reason, we sought to develop a series of ATP-competitive small molecule inhibitors that selectively inhibit ITK.

Several chemical series with potent inhibitory activities toward ITK have been discovered by various medicinal chemistry efforts, while only one has progressed into phase 2 clinical trials (NCT02919475). However, these molecules display relatively low specificity due to the conserved architecture of the ATP binding site of other TEC family enzymes and kinases in general. (Guo et al, Mol Pharmacol 82, 938-947 (2012)). One proven approach to improve specificity is to target a cysteine that is poorly conserved in the ATP site of kinases for covalent inhibition. This strategy has demonstrated clinical validation for the two protein tyrosine kinases, EGFR and BTK, and the KRAS Gly12Cys mutant GTPase involved in solid organ and blood cancers. In addition, there are programs in clinical trials targeting JAK3 and CDK7 using a similar covalent strategy (NCT03395184, NCT04247126). We exploited this approach to target Cys-442 in the hinge region of ITK for covalent interaction. To achieve the desired selectivity, we started with the 2-amino-5-(thioaryl)thiazole scaffold. Analysis of available crystal structures and computer modeling suggested that the methyl-methoxy-substituted aryl ring of this scaffold could interact with the Phe-435 and Ser-499 sidechains, which are unique to ITK. We preserved these interactions important for selectivity while designing and optimizing our inhibitors. This effort culminated in the discovery and characterization of CPI-818 which displays the highest specificity of a covalent ITK inhibitor reported to date.

A primary goal of our chemistry optimization effort was to potently and selectively inhibit ITK while sparing RLK since T cells from ITK null mice have only impaired activation and differentiation while ITK^{-/-}RLK^{-/-} double knockout T cells have a profound loss of normal T cell function. Thus, achieving significant selectivity over RLK will avoid potential effects on overall T-dependent immunity that might arise from chronic dosing of a broad TEC kinase or dual ITK/RLK inhibitor. The most informative measure of potency for an irreversible inhibitor is the second order rate constant of inactivation, k_{inact}/Kᵢ. We determined a k_{inact}/Kᵢ value of 1.3×10⁻² μM⁻¹ s⁻¹ for CPI-818 at ITK while at RLK the value was 1.13×10⁻⁴ μM⁻¹ s⁻¹. Thus CPI-818 is 115-fold selective toward ITK compared to RLK.

Not every kinase of the eleven that contain a homologous Cys in the ATP binding site is easily amenable to measurement of inactivation by CPI-818 and we resorted to an IC₅₀ measurement for this collection of enzymes. By this criterion the molecule was at least 80-fold selective over the ten other homologous Cys containing kinases. Evaluating selectivity even more broadly against the human kinome revealed that only the targeted kinase ITK was inhibited by >95% when tested at 1 μM.

Covalent ITK inhibitors targeting Cys-442 have been reported previously, however the molecules are not selective and inhibit TEC kinases or other kinases that harbor a homologous cysteine residue. Ibrutinib is a potent BTK inhibitor and a weaker ITK inhibitor. (Honigberg et al, Proc Natl Acad Sci USA 107, 13075-13080 (2010)). Although it has been reported as an irreversible molecular inhibitor or ITK it is relatively nonselective toward several other kinases and achieves only modest levels of ITK occupancy (50%) in PBMCs from subjects administered up to 840 mg ibrutinib. Pfizer described PF-06465469 with a k_{inact}/Kᵢ for ITK of 0.016 μM⁻¹ s⁻¹ which is 4-fold weaker than CPI-818 and was reported to be equally potent at BTK in an IC₅₀ assay. (Zapf et al, J Med Chem 55, 10047-10063 (2012)). GSK also produced a chemical series for inhaled delivery where a representative molecule was reported with k_{inact}/Kᵢ=1.9 μM⁻¹ s⁻¹ and BTK IC₅₀=80 nM. (Harling et al, J Biol Chem 288, 28195-28206 (2013)). More recently Principia described PRN694 as a dual ITK and RLK inhibitor with k_{inact}/Kᵢ values of 4.7 μM⁻¹ s⁻¹ and 0.46 μM⁻¹ s⁻¹, respectively. (Zhong et al, J Biol Chem 290, 5960-5978 (2015)). The molecule also displayed significant inhibition toward the other TEC kinases. Thus, among reported covalent kinase inhibitors, CPI-818 is the most selective irreversible ITK inhibitor characterized to date.

CPI-818 inhibited canonical functional effects of T cell activation in Jurkat and human CD4⁺ cells by selective blockade of ITK activity. The TCR proximal signaling molecules PLC-γ. ERK and ribosomal protein S6 are rapidly and transiently activated by phosphorylation following stimulation of the receptor and this phosphorylation was potently inhibited by CPI-818. IL-2 secretion is a downstream consequence of TCR engagement, and its accumulation was also strongly suppressed in Jurkat cultures treated with CPI-818. In an engineered functional assay measuring autophosphorylation, the specificity for ITK observed in biochemical assays was maintained in cells where CPI-818 was 40-fold selective over TEC and ≥80-fold over JAK3 and the other four members of the TEC family kinases.

By developing an ITK occupancy assay we gained valuable insight into the rate of its cellular turnover and the level of target engagement in animal models which enabled an initial PK/PD relationship for CPI-818 to be established. Using this probe-based method we found the rates of ITK degradation and synthesis were similar in the Jurkat cell line and in primary human T cells, where half-lives in both cell types for the opposing processes ranged between 6.6 to 8.2 h. These values differ from those reported previously. Using a pulse-chase method and ³⁵S Met/Cys in unstimulated primary human CD4⁺ cells, the half-life of ITK degradation was one hour which increased to 22 h when cells were treated with an irreversible ITK inhibitor. In contrast, another study using the same method in Jurkat cells stimulated with anti-CD3/CD28 found that cells treated with an irreversible ITK inhibitor increased the rate of the enzyme's degradation while in untreated cells the kinase had a long half-life. Neither study reported a rate of ITK synthesis. It is possible the differences between these studies lie in the different cell types and stimulation, but we found the Jurkat cell line and primary human T cells display similar ITK turnover rates in the unstimulated condition.

When a single dose of CPI-818 was administered to mice, essentially complete ITK occupancy was achieved 30 to 120 min following administration and was maintained at >87% during the first 8 h but decreased to 17% at 24 h. The initial blockade of the ITK pool present at the time of compound administration can eventually be overcome by synthesis of new kinase after clearance of the inhibitor. However, the production of uninhibited enzyme can be counteracted by more frequent dosing and our studies in mice and human T cells indicate the turnover rate of ITK requires BID dosing for continuous high levels of ITK occupancy by CPI-818.

Having established the potent and selective activity of CPI-818 in biochemical and functional assays and the molecules favorable drug attributes through in vivo studies we demonstrated the therapeutic potential of CPI-818 in a model of ulcerative colitis. The compound had a protective effect measured by gross anatomical end points where it maintained colon weights and lengths within the ranges of naïve animals. Histological evidence of disease severity was significantly reduced by treatment with CPI-818 to levels comparable to those of anti-IL-12/23 treatment and prevented infiltration of CD3+ T cells into lesions.

By exploiting covalent inhibition of ITK, our strategy benefits from several chemical design features including (1) potent irreversible inhibition that persists over the biological lifetime of the target, (2) introduction of a selectivity element that significantly reduces the complexity of unintended targets within a class of enzymes that has over 500 members sharing a conserved ATP binding site, (3) kinases that do not contain the targeted cysteine may be weakly inhibited by a reversible mode only briefly before drug clearance and (4) limited chemical reactivity of acrylamide in CPI-818 is comparable to the approved agent, ibrutinib. These features, that were incorporated into the design of CPI-818, may lead to superior safety, tolerability, and therapeutic properties. CPI-818 is currently being evaluated in a P1/1b study in patients with relapsed/refractory T cell lymphoma (NCT03952078).

### Example 2

A 57-year-old female patient with peripheral T-cell lymphoma NOS has multiple nodules in her neck, mediastinum abdomen, pelvis, and groin. CHOP therapy (combination chemotherapy with cyclophosphamide, doxorubicin, vincristine, and prednisolone) resulted in partial response for 5 months. The patient had an autologous stem cell transplant for progressive disease, and relapsed one year later. The patient was started on treatment with CPI-818 (200 mg BID) and achieved complete remission lasting 25 months.

### Example 3

A patient with lupus will be treated with CIP-818, and their symptoms will be reduced and the number of flares the patient experiences will be reduced compared to the number of flares the patient experiences without treatment with CPI-818. These results will be obtained because CPI-818 inhibits lymphadenopathy (FIG. 29A) and proteinuria (FIG. 29B) in mouse MRL lymphoproliferation strain (a.k.a. MRL/lpr^{-/-}) lupus model.

### Example 4

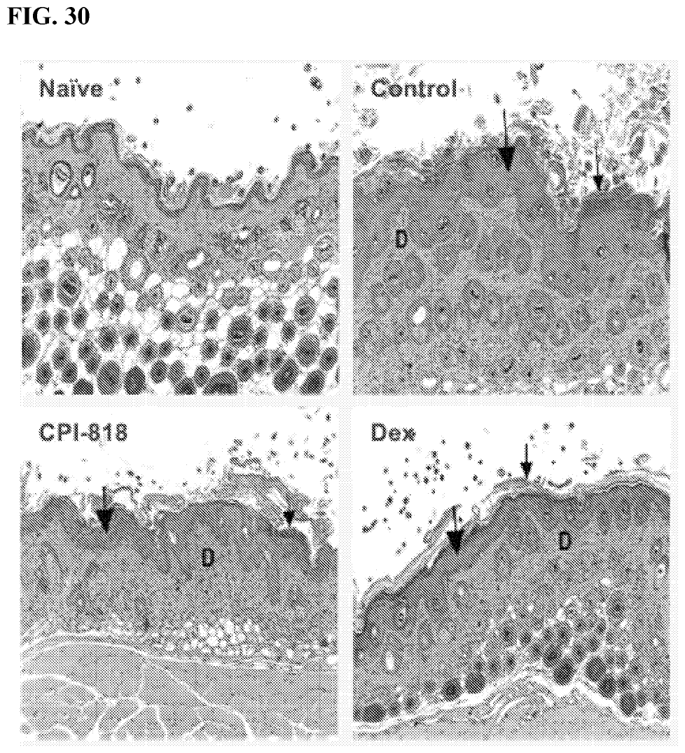

A patient with psoriasis will be treated with CPI-818, and they will experience significantly reduced skin thickening and dermal inflammation. These results are expected based on a imiquimod-induced model of psoriasis in which CPI-818 significantly reduced skin thickening and dermal inflammation (FIG. 30).

### Example 5

A study was conducted to show the effects of CPI-818 in a bleomycin-induced mouse pulmonary fibrosis model. Mice were administered bleomycin at day 0; oral QD dosing of mice with CPI-818, vehicle, or nintedanib began at day 7; and the mice were sacrificed at day 21. The treatment conditions are shown in the table below.

| Group | N | Challenge | Treatment | Description |
|---|---|---|---|---|
| 1 | 5 | PBS | Vehicle | Normal Lungs |
| 2 | 9 | Bleomycin | Vehicle | Negative Control |
| 3 | 10 | 1.5 U/kg | CPI-818 | High Dose Test Article |
| | | oropharyngeal | 30 mg/kg QD | |
| 4 | 10 | dose | CPI-818 | Low Dose Test Article |
| | | | 10 mg/kg QD | |
| 5 | 10 | | Nintedanib | Positive Cotrol |
| | | | 50 mg/kg QD | |

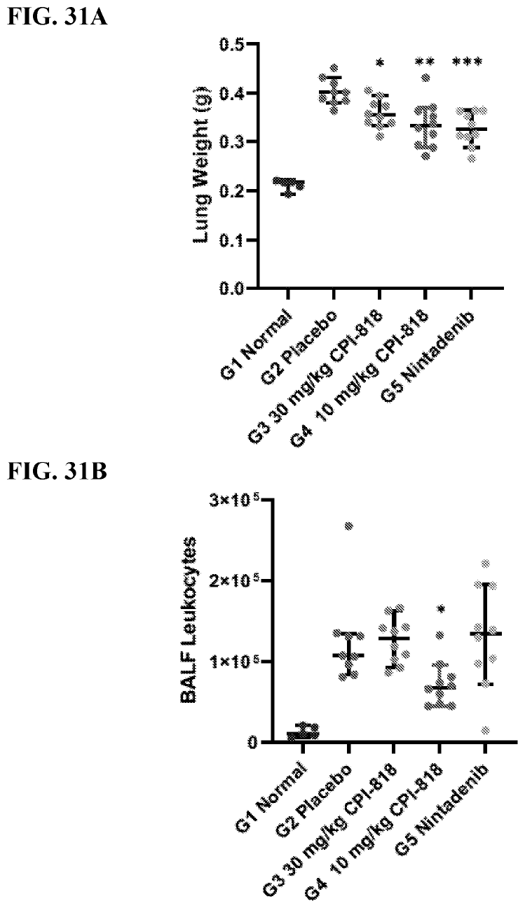

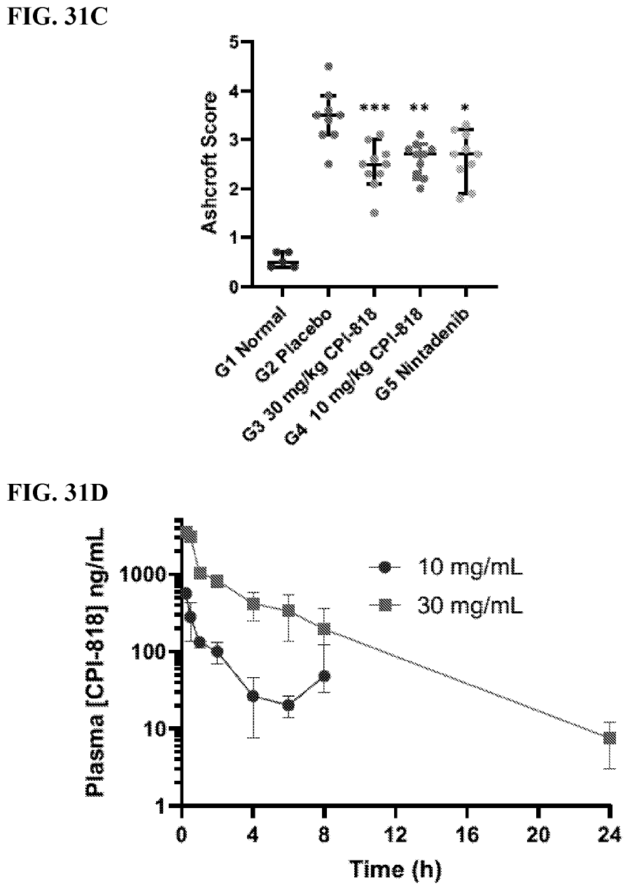

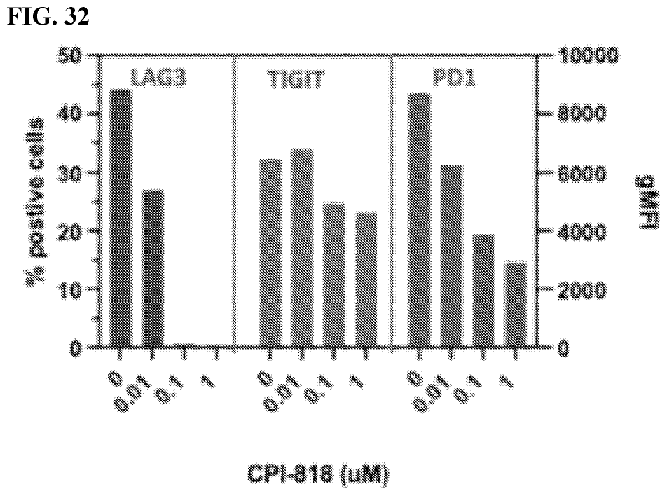

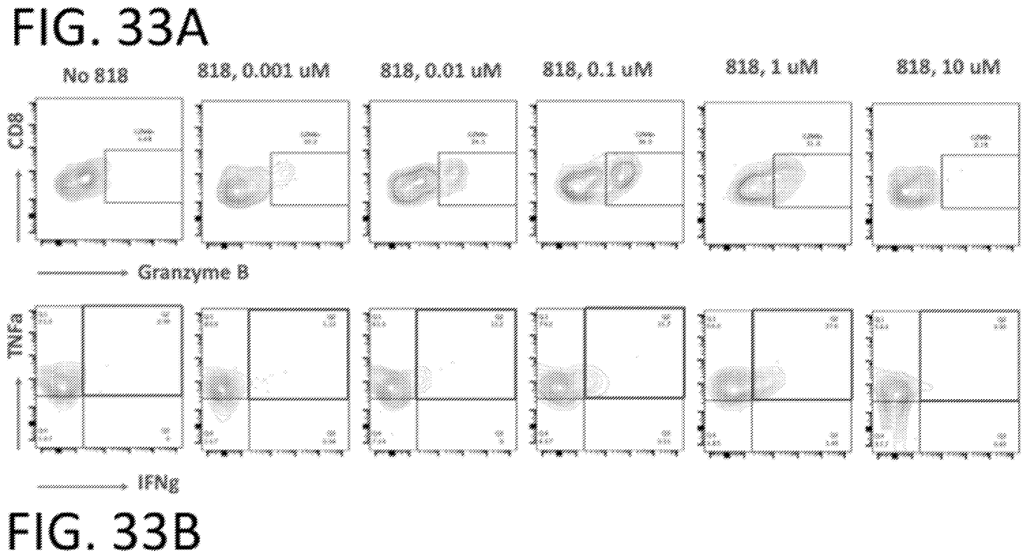

Following sacrifice, analysis was made of body weight, lung weight, BALF cell counts and differentials, and Ashcroft scoring (histological assessment of lung tissue by a veterinary pathologist blinded to treatment conditions. FIGS. 31A-31B show the positive impact of CPI-818 on lung weight and BALF leukocytes, respectively. FIG. 31C shows that CPI-818 reduced the Ashcroft score comparable to/better than nintedanib. FIG. 31D shows the plasma concentration of the 10 mg/mL and 30 mg/mL solutions of CPI-818 in mice.

The section headings used herein are for organizational purposes only and are not to be construed as limiting the subject matter described. All documents, or portions of documents, cited in the application are hereby expressly incorporated by reference in their entirety for any purpose.

While various embodiments and aspects are shown and described herein, it will be obvious to those skilled in the art that such embodiments and aspects are provided by way of example only. Numerous variations, changes, and substitutions will now occur to those skilled in the art. Various alternatives to the embodiments and aspects described herein may be used.

What is claimed is:

1. A method of treating a patient having deficient Th1 activity, the method comprising administering to the patient an ITK inhibitor at an effective amount to increase Th1 activity.

2. The method of claim 1, wherein the patient has increased Th2 activity relative to a control.

3. The method of claim 1, wherein the patient has an increased level of a pro-inflammatory cytokine relative to a control; wherein the pro-inflammatory cytokine is IL-4, IL-5, IL-10, IL-13, IL-17, or a combination of two or more thereof.

4. A method of treating a patient having deficient Th1 activity, the method comprising:
(i) measuring increased Th2 activity, an increased level of IL-4, an increased level of IL-5, an increased level of IL-10, an increased level of IL-13, an increased level of IL-17, a decreased level of IFNγ, a decreased number of Th1⁺ T cells, a decreased ratio of Th1⁺ T cells to Th2⁺ T cells, a decreased ratio of IFNγ⁺CD4⁺ T cells to IL-4⁺CD4⁺ T cells, a decreased number CD8+ cytotoxic lymphocytes, an increased number of Th2+ cells, an increased number of Th17+ T cells, an increased number of eosinophils, or any combination of two or more thereof, relative to a control, in a biological sample obtained from the patient; and
(ii) administering to the patient an ITK inhibitor at an effective amount to increase Th1 activity.

5. The method of claim 1, wherein the effective amount to increase Th1 activity is an amount that: (a) increases the number of Th1⁺ T cells; (b) increases the ratio of Th1⁺ T cells to Th2⁺ T cells; (c) increases the ratio of IFNγ⁺CD4⁺ T cells to IL-4⁺CD4⁺ T cells; (d) increases IFNγ production; (e) increases CD8+ cytotoxic lymphocytes; (f) inhibits IL-4 production; (g) inhibits IL-13 production; (h) decreases Th2+ cells; (i) decreases Th17+ T cells; (j) decreases eosinophils; (k) inhibits Th2; (1) inhibits the differentiation of naïve CD4 cells into Th2 cells; or (m) a combination of two or more of (a)-(j).

6. The method of claim 1, wherein the effective amount to increase Th1 activity is an amount that inhibits production of cytokines secreted by Th2+ cells.

7. The method of claim 1, wherein the effective amount to increase Th1 activity is about 0.6 mmole to about 1.0 mmole of the ITK inhibitor per day or about 0.3 mmole to about 0.8 mmole of the ITK inhibitor twice per day.

8. A method of treating a cancer, an autoimmune disease, or an allergy in a patient in need thereof, the method comprising administering to the patient about 0.6 mmole per day to about 1.6 mmole per day of an ITK inhibitor.

9. The method of claim 8, wherein the cancer is T-cell lymphoma, T cell leukemia, lung cancer, colorectal cancer, pancreatic cancer, prostate cancer, breast cancer, gastric cancer, or head and neck cancer; wherein the autoimmune disease is autoimmune lymphoproliferative disease, colitis, inflammatory bowel disease, or systemic lupus erythematosus; and wherein the allergy is asthma, dermatitis, rhinitis, or psoriasis.

10. A method of treating Th2/ITK-mediated disease in a patient in need thereof, the method comprising administering to the patient about 0.6 mmole per day to about 1.6 mmole per day of an ITK inhibitor.

11. A method of treating Th2/ITK-mediated disease in a patient in need thereof, the method comprising:
(i) measuring a decreased level of Th1⁺ T cells, a decreased ratio of Th1V T cells to Th2⁺ T cells; a decreased ratio of IFNγ⁺CD4⁺ T cells to IL-4⁺CD4⁺ T cells; a decreased level of IFNγ; a decreased level CD8+ cytotoxic lymphocytes; an increased level of Th2+ cells; an increased level of IL-4; an increased level of IL-5; an increased level of IL-10; an increased level of IL-13; an increased level of IL-17; an increased level of Th17+ T cells; an increased level of eosinophils; a decreased level of IL-1β, a decreased level of IL-2, a decreased level of IL-12, a decreased level of TNF-α, a decreased level of TNF-γ, a decreased level of GMCS, or a combination of two or more thereof, relative to a control, in a biological sample obtained from the subject, and
(ii) administering to the patient an effective amount of an ITK inhibitor.

12. The method of claim 11, wherein (i) comprises measuring an increased level of Th2+ cells; an increased level of IL-4; an increased level of IL-5; an increased level of IL-10; an increased level of IL-13; an increased level of IL-17; or a combination of two or more thereof, relative to a control, in the biological sample obtained from the subject.

13. The method of claim 12, wherein (i) comprises measuring an increased level of IL-4; an increased level of IL-5; an increased level of IL-10; an increased level of IL-13; an increased level of IL-17; or a combination of two or more thereof, relative to a control, in a biological sample obtained from the subject

14. The method of claim 11, wherein the Th2/ITK-mediated disease is atopic dermatitis, asthma, rhinitis, conjunctivitis, psoriasis, scleroderma, pulmonary fibrosis, cirrhosis, retroperitoneal fibrosis, psoriatic arthritis, vasculitis, autoimmune lymphoproliferative syndrome, chronic obstructive pulmonary disease, an eosinophilic disease, a mast cell disease, or human immunodeficiency viral disease.

15. The method of claim 1, wherein the ITK inhibitor is a compound of Formula (A) or a pharmaceutically acceptable salt thereof:

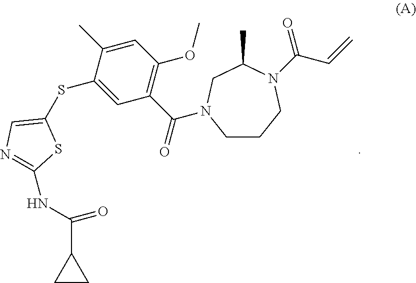 [SMILES: C=CC(=O)N1CCCN(C(=O)c2cc(Sc3cnc(NC(=O)C4CC4)s3)c(C)cc2OC)C[C@H]1C]

16. The method of claim 10, comprising administering to the patient about 0.6 mmole to about 1.0 mmole of the ITK inhibitor per day or about 0.3 mmole to about 0.5 mmole of the ITK inhibitor twice per day.

17. The method of claim 1, comprising the steps, in order:
(i) administering the ITK inhibitor for about 4 weeks to about 12 weeks;
(ii) discontinuing administration of the ITK inhibitor for about 1 week to about 8 weeks, and
(iii) administering the ITK inhibitor for at least four weeks.

18. A method for treating a cancer, an autoimmune disease, or an allergy in a patient in need thereof, the method comprising administering to the patient about 250 mg to about 1,000 mg per day of a compound of Formula (A) or a pharmaceutically acceptable salt thereof, wherein the compound of Formula (A) is:

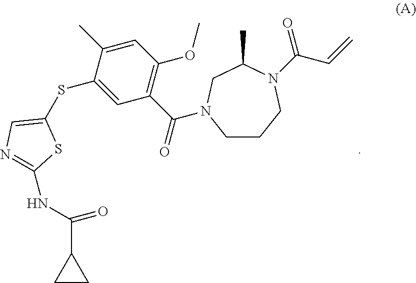 [SMILES: C=CC(=O)N1CCCN(C(=O)c2cc(Sc3cnc(NC(=O)C4CC4)s3)c(C)cc2OC)C[C@H]1C]

19. The method of claim 18 for treating the cancer; wherein the cancer is lymphoma, leukemia, lung cancer, colorectal cancer, pancreatic cancer, prostate cancer, breast cancer, gastric cancer, or head and neck cancer.

20. The method of claim 18 for treating the autoimmune disease; wherein the autoimmune disease is autoimmune lymphoproliferative disease, colitis, inflammatory bowel disease, or systemic lupus erythematosus.

21. The method of claim 18 for treating the allergy; wherein the allergy is allergic asthma, atopic dermatitis, allergic dermatitis, allergic rhinitis, or psoriasis.

22. A method for an Th2/ITK-mediated disease in a patient in need thereof, the method comprising administering to the patient about 250 mg to about 1,000 mg per day of a compound of Formula (A) or a pharmaceutically acceptable salt thereof, wherein the compound of Formula (A) is:

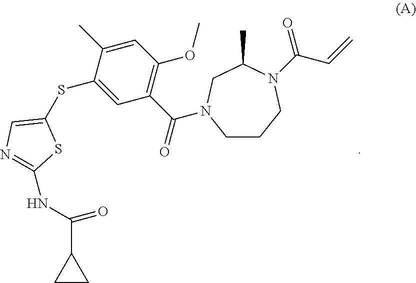 [SMILES: C=CC(=O)N1CCCN(C(=O)c2cc(Sc3cnc(NC(=O)C4CC4)s3)c(C)cc2OC)C[C@H]1C]

23. The method of claim 22, wherein the Th2/ITK-mediated disease is atopic dermatitis, asthma, rhinitis, conjunctivitis, psoriasis, scleroderma, pulmonary fibrosis, cirrhosis, retroperitoneal fibrosis, psoriatic arthritis, vasculitis, autoimmune lymphoproliferative syndrome, chronic obstructive pulmonary disease, an eosinophilic disease, a mast cell disease, or human immunodeficiency viral disease.

24. The method of claim 18, comprising administering to the patient about 250 mg to about 900 mg per day of the compound of Formula (A) or the pharmaceutically acceptable salt thereof or about 125 mg to about 500 mg twice per day per day of the compound of Formula (A) or the pharmaceutically acceptable salt thereof.

25. The method of claim 24, comprising administering to the patient about 400 mg per day of the compound of Formula (A) or the pharmaceutically acceptable salt thereof or about 200 mg twice per day of the compound of Formula (A) or the pharmaceutically acceptable salt thereof.

26. The method of claim 18, comprising the steps, in order:
(i) administering the compound of Formula (A) or the pharmaceutically acceptable salt thereof for about 4 weeks to about 12 weeks;
(ii) discontinuing administration of the compound of Formula (A) or the pharmaceutically acceptable salt thereof for about 1 week to about 8 weeks, and
(iii) administering the compound of Formula (A) or the pharmaceutically acceptable salt thereof for at least 4 weeks.

27. A pharmaceutical composition comprising about 200 mg of a compound of Formula (A) or a pharmaceutically acceptable salt thereof and a pharmaceutically acceptable excipient, wherein the compound of Formula (A) is:

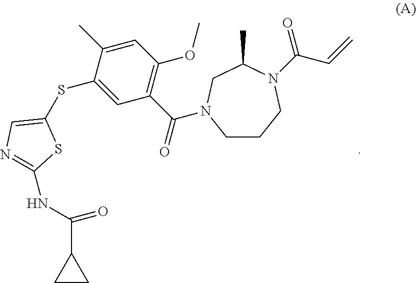 [SMILES: C=CC(=O)N1CCCN(C(=O)c2cc(Sc3cnc(NC(=O)C4CC4)s3)c(C)cc2OC)C[C@H]1C]

28. A pharmaceutical composition comprising about 400 mg of a compound of Formula (A) or a pharmaceutically acceptable salt thereof and a pharmaceutically acceptable excipient, wherein the pharmaceutical composition is in a single or divided dose, and wherein the compound of Formula (A) or the pharmaceutically acceptable salt thereof is:

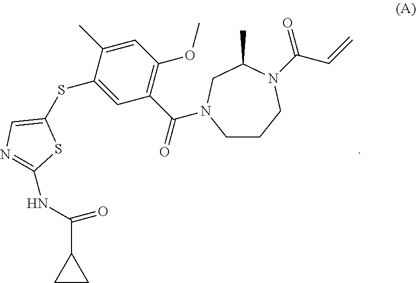 [SMILES: C=CC(=O)N1CCCN(C(=O)c2cc(Sc3cnc(NC(=O)C4CC4)s3)c(C)cc2OC)C[C@H]1C]

29. A method of reversing T cell exhaustion in a patient in need thereof, the method comprising administering to the patient an effective amount of an ITK inhibitor.

30. A method of reversing T cell exhaustion in a patient in need thereof, the method comprising:
(i) measuring an increased level of LAG3, an increased level of TIGIT, an increased level of PD-1, a decreased level of IFNγ, a decreased level of granzyme B, or a combination of two or more thereof, relative to a control, in a biological sample obtained from the patient; and
(ii) administering to the patient an effective amount of an ITK inhibitor.

31. The method of claim 30, wherein the T cell is a CD4 T cell.

32. A method of treating cancer in a patient in need thereof, the method comprising administering to the patient an effective amount of an ITK inhibitor; wherein the patient has T cell exhaustion.

33. A method of treating cancer in a patient in need thereof, the method comprising
(i) measuring an increased level of LAG3, an increased level of TIGIT, an increased level of PD-1, a decreased level of IFNγ, a decreased level of granzyme B, or a combination of two or more thereof, relative to a control, in a biological sample obtained from the patient; and
(ii) administering to the patient an effective amount of an ITK inhibitor.

34. The method of claim 33, wherein the cancer is lymphoma, leukemia, lung cancer, colorectal cancer, pancreatic cancer, prostate cancer, breast cancer, gastric cancer, renal cancer, or head and neck cancer.

35. The method of claim 33, wherein the effective amount is about 250 mg to about 900 mg per day or about 125 mg to about 500 mg twice per day per day.

36. The method of claim 35, wherein the effective amount is about 400 mg per day or about 200 mg twice per day.

37. The method of claim 33, wherein the ITK inhibitor is a compound of Formula (A) or a pharmaceutically acceptable salt thereof:

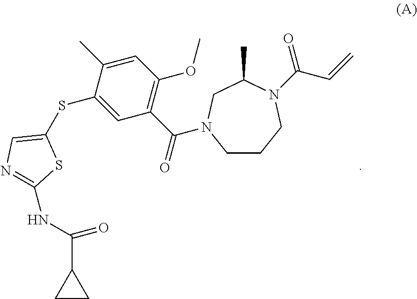 [SMILES: C=CC(=O)N1CCCN(C(=O)c2cc(Sc3cnc(NC(=O)C4CC4)s3)c(C)cc2OC)C[C@H]1C]

38. The method of claim 32, wherein the ITK inhibitor is a compound of Formula (I) or a pharmaceutically acceptable salt thereof compound having the formula: 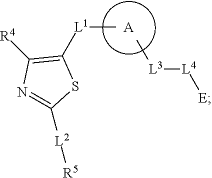
wherein,
Ring A is 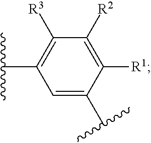
R¹ is independently hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is independently hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR©D, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ is —O—, —S—, or substituted or unsubstituted C₁-C₂ alkylene, or substituted or unsubstituted 2 membered heteroalkylene;
L² is a bond, —NH—, or —NHC(O)—;
L³ is a bond, —S(O)₂—, —N(R⁶)—, —O—, —S—, —C(O)—, —C(O)N(R⁶)—, —N(R⁶)C(O)—, —N(R⁶)C(O)NH—, —NHC(O)N(R⁶)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁶ is independently hydrogen, —CX⁶₃, —CHX⁶₂, —CH₂X⁶, —CN, —C(O)R^{6C}, —C(O)OR^{6C}, —C(O)NR^{6A}R^{6B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴ is substituted or unsubstituted heterocycloalkylene;
E is: 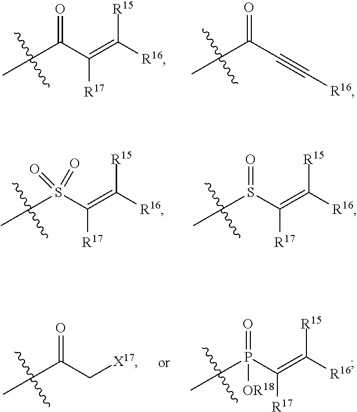
each R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{6A}, R^{6B}, and R^{6C} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{6A}, and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl;
each X, X¹, X², X³, X⁴, and X⁶ is independently —F, —Cl, —Br, or —I;
n1, n2, n3, and n4 are independently an integer from 0 to 2;
m1, m2, m3, m4, v1, v2, v3, and v4 are independently 1 to 2;
R¹⁵ is independently hydrogen, halogen, —CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁶ is independently hydrogen, halogen, —CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC═(O)NHNR^{16A}R^{16B}, —NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁷ is independently hydrogen, halogen, —CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, and R^{18C}, are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl;
each X, X¹⁵, X¹⁶, X¹⁷ and X¹⁸ is independently —F, —Cl, —Br, or —I;
n15, n16, and n17 are independently an integer from 0 to 2;
v15, v16, and v17 are independently 1 or 2; and
m15, m16, and m17 are independently 1 or 2.

39. The method of claim 32, wherein the ITK inhibitor is a compound of Formula (II) or a pharmaceutically acceptable salt thereof, having the formula: 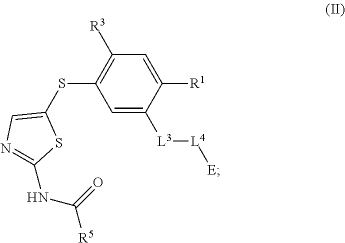
wherein:
R¹ is hydrogen, halogen, —CX¹₃, —CHX¹, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —CN, —SO₁₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, unsubstituted or substituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is unsubstituted or substituted cycloalkyl, substituted or unsubstituted alkyl, substituted or unsubstituted heteroalkyl, substituted or unsubstituted heterocycloalkyl, substituted or unsubstituted aryl, or substituted or unsubstituted heteroaryl;
L³ is —C(O)—, a bond, —N(R⁶)—, or —C(O)N(R⁶)—;
R⁶ is hydrogen or methyl;
L⁴ is substituted or unsubstituted 5 to 8 membered monocyclic heterocycloalkylene;
E is —C(O)CH═CH₂;
R^{1A}, R^{1B}, R^{1C}, R^{1D}, R^{3A}, R^{3B}, R^{3C}, and R^{3D} are each independently hydrogen, —CX₃, —CN, —COOH, —CONH₂, —CHX₂, —CH₂X, unsubstituted alkyl, unsubstituted heteroalkyl, unsubstituted cycloalkyl, unsubstituted heterocycloalkyl, unsubstituted aryl, or unsubstituted heteroaryl;
X, X¹, and X³ are each independently —F, —Cl, —Br, or —I;
n1 and n3 are independently an integer from 0 to 4; and
m1, m3, v, and v3 are independently 1 or 2.
